(19) United States
(12) Patent Application Publication
(10) Pub. No.: US 2026/0256873 A1
(43) Pub. Date: Sep. 3, 2026

(54) MODIFIED CAVEOLIN-1 PEPTIDE FORMULATIONS AND METHODS OF MANUFACTURING AND USE THEREOF

(71) Applicant: LUNG THERAPEUTICS, INC., Austin, TX (US)

(72) Inventors: John J. KOLENG, Austin, TX (US); Brian WINDSOR, Austin, TX (US); BreAnne MACKENZIE, Austin, TX (US); Dale J. CHRISTENSEN, Austin, TX (US)

(21) Appl. No.: 18/993,188

(22) PCT Filed: Jul. 12, 2023
(86) PCT No.: PCT/US2023/070053

§ 371 Date: Jan. 10, 2025

Related U.S. Application Data
(60) Provisional application No. 63/368,169, filed on Jul. 12, 2022.

(51) Int. Cl. A61K 38/10; A61K 38/08

(52) U.S. Cl. CPC A61K 38/10; A61K 38/08

(57) ABSTRACT

Provided herein are modified caveolin-1 (Cav-1) peptide and pharmaceutical formulations and methods of using the peptide or formulations for treating a subject in need thereof. In particular, provided herein are modified Cav-1 peptide formulations and methods of using the formulations for treating various diseases, such as fibrosis, by providing extended release of the peptide over a desired time period.

### CROSS REFERENCE TO RELATED APPLICATIONS

This application claims the benefit of priority to U.S. Provisional Application No. 63/368,169, filed Jul. 12, 2022, the content of which is incorporated by reference in its entirety.

### FIELD OF THE DISCLOSURE

The present disclosure relates to extended-release formulations comprising modified caveolin-1 peptides. The present disclosure also provides methods of using such formulations for the treatment of various diseases, including but not limited to fibrosis.

### INCORPORATION OF THE SEQUENCE LISTING

The Sequence Listing associated with this application is provided in eXtensible Markup Language (XML) format in lieu of a paper copy and is hereby incorporated by reference into the specification. A computer readable format copy of the Sequence Listing (file name: LUTX_025_01WO_SeqList_ST26; date of creation: Jul. 6, 2023; file size: 162,438 bytes) is being submitted electronically.

### BACKGROUND

Caveolin-1 (Cav-1) is an integral membrane protein that has homeostatic function in the fibrosis process by participating in a series of key regulatory pathways, such as TGF-0 signaling (Shihata et al., Front. Pharmacol. 2017; 8:567; and Gvaramia et al., Matrix Biol, 2013:32(6):307-315). Endogenous caveolin-1 was constitutively suppressed in fibrotic lungs in a bleomycin-induced animal model of IPF and IPF patients (Wang et al., J Exp Med, 2006; 203(13):2895-2906; Sanders et al., PLoS One, 2015; 10(2), e0116995; and Sanders et al., Am J Respir Cell Mol Biol, 2017; 56(1):50-61). Full-length caveolin-1 protein may be a less desirable pharmaceutical candidate due to inherent concerns for protein drugs, including stability, delivery, cost, and autoimmunogenicity. Therefore, fragments of caveolin-1, such as the caveolin scaffolding domain peptides (CSPs), have been studied and found to be effective substitutes for the full-length caveolin-1 protein. In particular, the 20-mer form of CSP has been shown to prevent, limit, or reverse fibrosis in animal models, and a seven amino acid fragment of CSP named CSP-7 was sufficiently effective in promoting the reduction of fibrosis both in vitro and in vivo (Marudamuthu et al., Sci Transl Med, 2019; 11(522), eaat2848). As a result, CSPs have been identified as promising therapeutic agents for the treatment of fibrotic diseases such as IPF.

Peptide therapeutics generally have short half-lives in vivo, requiring frequent administration of the therapeutics. Thus, there is a need for an extended-release formulation for modified Cav-1 peptide therapeutics to reduce frequent administration burden as well as minimizing adverse effects caused by peak-to-through fluctuations in serum drug concentration.

### SUMMARY OF THE DISCLOSURE

The present disclosure provides formulations that comprise a modified caveolin-1 peptide and at least one pharmaceutically acceptable carrier or an excipient.

In one aspect, the formulation comprises a polypeptide comprising an amino acid sequence having a core sequence of FTTFTVT and a sucrose acetate isobutyrate (SAIB).

In some embodiments, the formulation comprising SAIB further comprises a first solvent selected from N-methylpyrrolidone (NMP), anhydrous ethanol, or ethyl acetate. In some embodiments, the first solvent is NMP or ethyl acetate. In some embodiments, the SAIB is present in the first solvent in about 50% w/w to about 95% w/w or 70% w/w to about 90% w/w. In some embodiments, the SAIB is present in the first solvent in about 80% w/w.

In some embodiments of the formulation comprising SAIB, the polypeptide is micronized. In some embodiments, the polypeptide has a mean particle size in the range of about 0.5 μm to about 100 μm. In some embodiments, the polypeptide has a mean particle size in the range of about 1 μm to about 5 μm.

In some embodiments, the formulation comprising SAIB is in the form of an emulsion, a solution, microspheres, nanoparticles, nanospheres, implants, or gels. In some embodiments, the formulation is a suspension.

In some embodiments, the formulation comprising SAIB further comprises a second solvent. In some embodiments, the second solvent is sterile water, phosphate-buffered saline (PBS), or any pharmaceutically acceptable carrier.

In some embodiments, the formulation comprises the polypeptide and the SAIB at a weight ratio of about 1:50 to about 1:1. In some embodiments, the weight ratio is about 1:30 to about 1:1.

In some embodiments, the formulation comprising the polypeptide and the SAIB releases at least about 1% to about 30% of the polypeptide by four hours under physiological conditions. In some embodiments, the formulation releases at least about 5% to 25% of the polypeptide by four hours under physiological conditions. In some embodiments, the formulation comprising the polypeptide and the SAIB releases at least about 1% to about 30% or at least about 5% to 25% of the polypeptide by four hours when measured in an aqueous buffer in the range of about pH 6 to about pH 8 at about 37° C. In some embodiments, the formulation comprising the polypeptide and the SAIB releases at least about 1% to about 30% or at least about 5% to about 25% of the polypeptide by four hours when measured in an aqueous buffer of about pH 7.4 at about 37° C.

In some embodiments of any one of the SAIB formulations as described herein, the polypeptide comprises ASFTTFTVTK. In some embodiments, the polypeptide consists of 20 or less amino acids. In some embodiments, polypeptide comprises at least one amino acid added to the N-terminus; at least one amino acid added to the C-terminus; or at least one amino acid added to the N-terminus and the C-terminus. In these embodiments the addition made within 5 amino acids of each terminus has an amino acid sequence that is less than 80% identical to a contiguous amino acid sequence of SEQ ID NO:1 or 2 (human Cav-1 peptide).

In some embodiments, the addition made within 5 amino acids of each terminus has an amino acid sequence that is less than 60% identical to a contiguous amino acid sequence of SEQ ID NO: 1 or 2. In some the addition made within 5 amino acids of each terminus has an amino acid sequence that is less than 40% identical to a contiguous amino acid sequence of SEQ ID NO: 1 or 2. In some embodiments, the addition made within 5 amino acids of each terminus has an amino acid sequence that is less than 20% identical to a contiguous amino acid sequence of SEQ ID NO: 1 or 2. In some embodiments, the polypeptide sequence comprises the peptide comprises L-amino acids; the polypeptide comprises D-amino acids; or the peptide comprises both L- and D-amino acids.

In some embodiments, the polypeptide comprises at least one non-standard amino acid; or the polypeptide comprises two non-standard amino acids. In some embodiments, the non-standard amino acid is ornithine. In some embodiments, the polypeptide further comprises an N-terminal modification; a C-terminal modification; or an N- and C-terminal modification. In some embodiments, the N-terminal modification is acylation; and/or the C-terminal modification is amidation.

In some embodiment of any one of the SAIB formulations as described herein, the polypeptide comprises the amino acid sequence of FTTFTVT (SEQ ID NO: 3), KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10). In some embodiments, the polypeptide consists of an amino acid sequence selected from FTTFTVT (SEQ ID NO: 3), KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10). In some embodiments, the polypeptide consists of an amino acid sequence of Ac-aaEGKASFTTFTVTKGSaa-NH2 (SEQ ID NO: 8).

In yet another aspect, the present disclosure provides a method of treating a disease in a subject in need thereof, comprising administering to the subject a pharmaceutically effective amount of any one of the formulations as described herein. In some embodiments, the disease is fibrosis. In some embodiments, the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

In some embodiments, the interstitial lung disease is idiopathic pulmonary fibrosis, familial pulmonary fibrosis, idiopathic nonspecific interstitial pneumonia, conventional interstitial pneumonia, cryptogenic organizing pneumonia, or sarcoidosis. In some embodiments, the interstitial lung disease is idiopathic pulmonary fibrosis.

In some embodiments, the disease is a kidney disease or disorder. In some embodiments, the kidney disease or disorder is selected from the group consisting of chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, granulomatosis with polyangiitis, or acute kidney injury. In some embodiments, the kidney disease or disorder is kidney fibrosis. In some embodiments, the kidney disease or disorder is Alport syndrome. In some embodiments, the administration is intraocular, intradermal, transdermal, intramuscular, or subcutaneous administration. In some embodiments, the subject is a human.

In some embodiments of any one of the formulations as described herein, the formulation releases the peptide for at least 7 days following administration of a single dose.

In some embodiments, the formulation releases the polypeptide for at least 21 days following administration of a single dose. In some embodiments, the formulation releases the polypeptide for at least 28 days following administration of a single dose. In some embodiments, the formulation is administered to the subject at a dose of about 0.01 mg/kg to about 250 mg/kg.

In some embodiments, the formulation is administered to the subject at a dose of about 0.05 mg/kg to about 50 mg/kg.

It is contemplated that any method or formulation described herein can be implemented with respect to any other method or formulation described herein. Other objects, features, and advantages of the present disclosure will become apparent from the following detailed description. It should be understood, however, that the detailed description, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

FIG. 1 illustrates release (%) of the modified Cav-1 peptide (APi2335, SEQ ID NO: 8) over time (h) from an exemplary SAIB formulation.

FIG. 2 illustrates the mean plasma concentrations of the modified Cav-1 peptide (APi2355, SEQ ID NO: 8) in rat tissues following administration of an exemplary SAIB formulation.

### DETAILED DESCRIPTION

The present disclosure overcomes challenges associated with current technologies by providing modified caveolin-1 (Cav-1) peptides in controlled drug release dosage forms. In some embodiments, pharmaceutical formulations of the modified Cav-1 peptides are provided.

In some embodiments, the peptide is formulated for subcutaneous delivery. In some embodiments, the peptide is formulated for extended release. Also provided herein is a method of treating or preventing fibrosis or chronic kidney disease by administering to the subject a therapeutically effective amount of any one of the modified Cav-1 peptide formulations as described herein.

### I. Definitions

As used herein, the articles “a” or “an” refers to one or more than one of the grammatical object of the article. As used herein in the claim(s), when used in conjunction with the word “comprising,” the articles “a” or “an” refer to one or more than one of the grammatical object of the article.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” as used herein indicates that a value includes the inherent variation of error for the device, the method being employed to determine the value, the variation that exists among the study subjects, or a value that is within 10% of a stated value.

The term “peptide” or “polypeptide” refers to a sequence of amino acids made up of a single chain of amino acids joined by peptide bonds. The term may be used interchangeably with “protein” in its broadest sense to refer to a molecule of two or more amino acids, amino acid analogs, or peptidomimetics. In some embodiments, the amino acids are linked by peptide bonds. In some embodiments, the amino acids are linked by other types of bonds, e.g., ester, ether, etc. As used herein the term “amino acid” refers to either natural and/or unnatural or synthetic amino acids, including glycine and both the D or L optical isomers, and amino acid analogs and peptidomimetics.

Generally, peptides or polypeptides contain at least two amino acid residues and are less than about 50 amino acids (for example, 40 amino acids, 30 amino acids, 20 amino acids, or any numbers therein) in length, unless otherwise defined. In some embodiments, a peptide or polypeptide is provided with a counterion. In some embodiments, a peptide or polypeptide comprises a N- and/or C-terminal modification such a as blocking modification that reduced degradation.

The term “peptidomimetic” or “peptide mimic” means that a peptide according to the invention is modified in such a way that it includes at least one non-peptidic bond such as, for example, urea bond, carbamate bond, sulfonamide bond, hydrazine bond, or any other covalent bond. A peptide of three or more amino acids is commonly called an oligopeptide if the peptide chain is short. If the peptide chain is long, the peptide is commonly called a peptide or a protein.

In some embodiments, a peptide or peptide has a certain percentage (for example, 80%, 85%, 90%, or 95%) of “sequence identity” or “homology” to another sequence meaning that, when aligned, that percentage of amino acids are the same in comparing the two sequences. In some embodiments, the term “identity” or “homology” refers to the percentage of amino acid residues in the candidate sequence that are identical with the residue of a corresponding sequence to which it is compared after aligning the sequences and introducing gaps, if necessary, to achieve the maximum percent identity for the entire sequence, and not considering any conservative substitutions as part of the sequence identity. In some embodiments, N- or C-terminal extensions or insertions are not construed as reducing identity or homology. Alternatively, in some embodiments, N- or C-terminal extensions or insertions render the newly formed sequence to be not homologous to the original sequence. Alignment and the percent homology or sequence identity can be determined using software programs known in the art, for example those described in Current Protocols In Molecular Biology (F. M. Ausubel et al., eds., 1987) Supplement 30, section 7.7.18, Table 7.7.1.

The term “insertions” or “deletions” are typically in the range of about 1 to 5 amino acids. The variation allowed can be experimentally determined by producing the peptide synthetically while systematically making insertions, deletions, or substitutions of nucleotides in the sequence using recombinant DNA techniques.

The term “substitution” when referring to a peptide or polypeptide, refers to a change in an amino acid for a different entity, for example another amino acid or amino acid moiety. Amino acid substitutions include alterations in which an amino acid is replaced with a different naturally occurring or a non-conventional amino acid residue. Such substitutions may be classified as “conservative,” in which case an amino acid residue contained in a peptide is replaced with another naturally occurring amino acid of similar character either in relation to polarity, side chain functionality or size. Such conservative substitutions are well known in the art. Substitutions encompassed by the present invention may also be “non-conservative,” in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative. In some embodiments, the amino acid substitutions are non-conservative.

An “analog” of a molecule such as a peptide refers to a molecule similar in function to either the entire molecule or to a fragment thereof. The term “analog” is also intended to include allelic species and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N- or C-terminal amino acids. Examples ofunnatural amino acids include, but are not limited to disubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, and σ-N-methylarginine. Fragments and analogs can be screened for prophylactic or therapeutic efficacy in transgenic animal models as described below.

The term “covalently bonded” refers to a peptide or polypeptide joined either directly or indirectly (e.g., through a linker) by a covalent chemical bond. In some embodiments, the fusion peptides are covalently bonded.

The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single peptide harboring all the intended proteins. The order of arrangement of the proteins can vary. Fusion proteins can include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AU1, AU5, and avidin. Half-life extenders include Fc domain and serum albumin.

A “biologically active” caveolin-1 (Cav-1) peptide refers to a polypeptide that increases p53 protein levels, reduces urokinase plasminogen activator (uPA) and uPA receptor (uPAR), and/or increases plasminogen activator inhibitor-1 (PAI-1) expression in cells, such as fibrotic lung fibroblasts. In some embodiments, the biologically active peptide has at least 20% of the biological or biochemical activity of native Cav-1 peptide of SEQ ID NO: 1 (e.g., as measured by an in vitro or an in vivo assay). In some embodiments, the biological active peptide has an increase biological or biochemical activity as compared to the native Cav-1 peptide.

The term “isolated”, as used herein, refers to a peptide or polypeptide that has been separated from any natural environment, such as a body fluid, e.g., blood, and separated from the components that naturally accompany the peptide.

As used herein, “essentially free,” in terms of a specified component, means that none of the specified component has been purposefully formulated into a formulation or composition and/or is present only as a contaminant or in trace amounts. The total amount of the specified component resulting from any unintended contamination of a formulation is therefore well below 0.01%. Most preferred is a formulation or formulation in which no amount of the specified component can be detected using standard analytical methods.

The term “substantially pure” refers to a peptide or polypeptide that has been isolated and purified to at least some degree from the components that naturally accompany it. Typically, a peptide or peptide is substantially pure when it is at least about 60%, at least about 70%, at least about 80%, at least about 90%, at least about 95%, or at least about 99%, by weight, free from the proteins and naturally occurring organic molecules with which it is naturally associated. For example, a substantially pure peptide or peptide may be obtained by extraction from a natural source, by expression of a recombinant nucleic acid in a cell that does not normally express that protein, or by chemical synthesis.

The terms “subject” and “individual” and “patient” are used interchangeably herein, and refer to an animal, for example a human or non-human animal (e.g., a mammal), to whom treatment, including prophylactic treatment, with a modified Cav-1 peptide or pharmaceutical formulation thereof, as disclosed herein, is provided. The term “subject” as used herein refers to human and non-human animals. The term “non-human animals” includes all vertebrates, e.g., mammals, such as non-human primates (particularly higher primates), sheep, dogs, rodents (e.g. mouse or rat), guinea pigs, goats, pigs, cats, rabbits, cows, and non-mammals such as chickens, amphibians, reptiles, etc. In some embodiments, the subject is human. In some embodiments, the subject is an experimental animal or animal substitute as a disease model. Non-human mammals include mammals such as non-human primates (particularly higher primates), sheep, dogs, rodents (e.g., mouse or rat), guinea pigs, goats, pigs, cats, rabbits and cows. In some embodiments, the non-human animal is a companion animal such as a dog or a cat.

“Treating” a disease or condition in a subject or “treating” a patient having a disease or condition refers to subjecting the individual to a pharmaceutical treatment, e.g., the administration of a drug, such that at least one symptom of the disease or condition is decreased or stabilized. Typically, when a peptide of the present disclosure is administered therapeutically as a treatment, it is administered to a subject who presents with one or more symptoms of pathogen-induced lung injury.

The term “variant” as used herein refers to a peptide or nucleic acid that differs from the peptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally occurring or a non-conventional amino acid residue. Such substitutions may be classified as “conservative,” in which case an amino acid residue contained in a peptide is replaced with another naturally occurring amino acid of similar character either in relation to polarity, side chain functionality or size. Such conservative substitutions are well known in the art. Substitutions encompassed by the present invention may also be “non-conservative,” in which an amino acid residue which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions are conservative. Also encompassed within the term variant when used with reference to a polynucleotide or peptide, refers to a polynucleotide or peptide that can vary in primary, secondary, or tertiary structure, as compared to a reference polynucleotide or polypeptide, respectively (e.g., as compared to a wild-type polynucleotide or polypeptide).

The term “micronized” refers to a substance having a size of few microns.

The phrase “effective amount” or “therapeutically effective amount” means a dosage of a drug or agent sufficient to produce a desired therapeutic result. The desired therapeutic result can be subjective or objective improvement in the recipient of the dosage, reduced infection, reduced inflammation, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, “excipient” refers to pharmaceutically acceptable carriers that are relatively inert substances used to facilitate administration or delivery of an Active Pharmaceutical Ingredient (API) into a subject or used to facilitate processing of an API into drug formulations that can be used pharmaceutically for delivery to the site of action in a subject. Excipients or pharmaceutically acceptable carriers include all of the inactive components of the dosage form except for the active ingredient(s). Non-limiting examples of excipients include carrier agents, bulking agents, stabilizing agents, surfactants, surface modifiers, solubility enhancers, buffers, encapsulating agents, antioxidants, preservatives, nonionic wetting or clarifying agents, viscosity increasing agents, and absorption-enhancing agents. “Excipient free” refers to the modified Cav-1 peptide pharmaceutical formulation free of any excipients.

The term “physiological conditions” refer to a set of conditions including temperature, salt concentration, pH that mimic those conditions of a living subject. The conditions include physiologically relevant conditions for use in in vitro assays. Generally, a physiological buffer contains a physiological concentration of salt and at adjusted to a neutral pH ranging from about 6 to about 8, from about 6.5 to about 7.8, or from about 7.0 to about 7.5. Physiologically relevant temperature ranges from about 25° C. to about 38° C., or from about 30° C. to about 37° C. In some embodiments, the physiological buffer is a phosphate buffer, such as about 10 to about 100 mM phosphate buffer or phosphate buffered saline, or simulated body fluid. In some embodiments, the physiological buffer is rendered isotonic (such as about 250 to about 350 mOsm/kg) with soluble materials, including but not limited to, sodium chloride or dextrose. In some embodiments, the physiological buffer can also comprise a surfactant.

The phrases “pharmaceutical formulation”, “pharmaceutically acceptable formulation” or “pharmacologically acceptable formulation” refers to molecular entities and formulations that do not produce an adverse, allergic, or other untoward reaction when administered to an animal, such as a human, as appropriate. The preparation of a pharmaceutical formulation comprising a modified Cav-1 peptide, such as CSP7, or additional active ingredients will be known to those of skill in the art in light of the present disclosure. Moreover, for animal (e.g., human) administration, it will be understood that preparations should meet bioburden, sterility, pyrogenicity, general safety, and/or purity standards as required by the FDA or other recognized regulatory authority.

As used herein, “pharmaceutically acceptable carrier” includes any and all excipients, processing aids, aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, disintegration agents, lubricants, flavor modifiers (e.g., sweetening agents, flavoring agents), such like materials and combinations thereof, as would be known to one of ordinary skill in the art. The pH and exact concentration of the various components in a pharmaceutical formulation are adjusted according to well-known parameters. In some embodiments, the carrier may encapsulate a therapeutic agent, but not itself be consumed or administered to a subject (e.g., a shell capsule encasing a dry powder formulation, such as for use in a dry powder inhaler). See, e.g., Remington's Pharmaceutical Sciences, 18th Ed., 1990, incorporated herein by reference.

### II. Caveolin-1 Peptides

Embodiments of the present disclosure provide modified versions of the native caveolin-1 (Cav-1) protein, including, but not limited to, fragments, derivatives, and variants of the native Cav-1 protein. In some embodiments, the modified Cav-1 peptides are truncations of the native Cav-1 peptide, such as the exemplary peptides shown in Table 2 and/or Table 3.

Native human Cav-1 is 178 amino acids in length (see, SEQ ID NO: 1 in Table 1 below) and has a molecular weight of 22 kDa. Caveolin-1 is an integral membrane protein associated with endocytosis, extracellular matrix organization, cholesterol distribution, cell migration, and signaling. See, Boscher and Nabi, Adv Exp Med Biol, 2012;729-29-50.

TABLE 1
Amino Acid Sequences of Native Human Cav-1 and Cav-1 Scaffolding Domain

| | | SEQ ID |
|---|---|---|
| Name | Amino acid sequence | NO |
| Native human Cav-1 | MSGGKYVDSEGHLYTVPIREQGNIYKPNNKA | 1 |
| | MADELSEKQVYDAHTKEIDLVNRDPKHLND | |
| | DVVKIDFEDVIAEPEGTHSFDGIWKASFTTFT | |
| | VTKYWFYRLLSALFGIPMALIWGIYFAILSFL | |
| | HIWAVVPCIKSFLIEIQCISRVYSIYVHTVCDPL | |
| | FEAVGKIFSNVRINLQKEI | |
| Cav-1 scaffolding | DGIWKASFTTFTVTKYWFYR | 2 |
| domain | | |

In some embodiments, the modified Cav-1 peptide is the Cav-1 scaffolding domain (CSD). The CSD is comprised of the amino acids 82-101 of caveolin-1 (see, SEQ ID NO: 2 in Table 1 above). The CSD of caveolin-1 plays a critical role in caveolin-1 dimerization as well as regulation of diverse signaling intermediates (Shetty et al., Am J Respir Cell Mol Biol 2012; 47:474-83; Fridolfsson et al., FASEB J 2014; 28:3823-31; Degryse et al., Am J Physiol Cell Mol Physiol 2010; 299: L442-L452; and Egger et al., PLos One, 2013; 8:e63432). The CSD domain of caveolin-1 has demonstrated inhibition of Wnt-signaling, β-catenin-mediated transcription, activation of SRC, EGFR, MEK1 and ERK-2 and various other factors (see, Shetty et al., Am J Respir Cell Mol Biol 2012; 47:474-83; Bhandary et al., Am J Physiol Cell Mol Physiol 2012; 302:L463-L473; Bhandary et al., Am J Pathol 2013; 183:131-143; Fridolfsson et al., FASEB J 2014; 28:3823-31; Degryse et al., Am J Physiol Cell Mol Physiol 2010; 299:L442-L452; and Fiddler et al., Ann Am Thorac Soc, 2016; 13:1430-2). For example, the CSD of Cav-1 interferes with Cav-1 interaction with SRC kinases and mimics the combined effect of uPA and anti-β1-integrin antibody. Endogenous CSD domains can form homodimers with other Cav-1 proteins and interact with proteins that have a caveolin binding domain sequence (CBD) motif It is estimated that up to 30% of all endogenous proteins have CBD motifs and the caveolin-1 CSD domain is hypothesized to provide stability to these proteins (see, Marudamuthu et al., Am J Pathol 2015; 185:55-68). Treatment with the CSP 20-mer (the full-length CSD of caveolin-1) resulted in reduced lung αSMA and lung epithelial apoptosis, reduced collagen deposition, and down-regulation of expression of profibrogenic signaling molecules (see, Bhandary et al., Am J Phys Lung Cell Mol Phys, 2012, 302(5), L463-L473; Razani et al., J B C, 2001, 276(9),6727-6738; and Lee et al., Biochem Biophys Res Commun, 2007, 359(2): 385-390).

In some embodiments, modified Cav-1 peptide is CSP-7. CSP-7 is a seven amino acid fragment of the human CSD of caveolin-1 (see, SEQ ID NO: 3 in Table 3 below).

Exemplary amino acid sequences of the modified Cav-1 peptides are shown below in Tables 2 and 3. Upper case letters denote L-amino acids and lower-case letters denote D-amino acids (e.g., lowercase “a” represents D-alanine). The term “Ac” refers to an acetyl group and the term “NH2” refers to an amido group. The “O” denotes ornithine.

TABLE 2
Illustrative Modified Cav-1 Peptides

| | SEQ ID |
|---|---|
| Amino Acid Sequence | NO |
| KASFTTFTVTKGS | 4 |
| KASFTTFTVTKGS-NH2 | 5 |
| aaEGKASFTTFTVTKGSaa | 6 |
| aaEGKASFTTFTVTKGSaa-NH2 | 7 |
| Ac-aaEGKASFTTFTVTKGSaa-NH2 | 8 |
| OASFTTFTVTOS | 9 |
| OASFTTFTVTOS-NH2 | 10 |
| FTTFTVT-NH2 | 11 |
| FTTFTVTK-NH2 | 12 |
| KASFTTFTVTK-NH2 | 13 |
| Ac-KASFTTFTVTK-NH2 | 14 |
| OASFTTFTVTK-NH2 | 15 |
| Ac-OASFTTFTVTK-NH2 | 16 |
| Ac-KASFTTFTVTKGS-NH2 | 17 |
| DSGKASFTTFTVTK-NH2 | 18 |
| Ac-DSGKASFTTFTVTK-NH2 | 19 |
| Ac-OASFTTFTVTOS-NH2 | 20 |

TABLE 3
Additional Illustrative Modified Cav-1 Peptides

| | SEQ ID | | SEQ ID |
|---|---|---|---|
| Amino acid sequence | NO | Amino acid sequence | NO |
| FTTFTVT | 3 | ASFTTFTVTK | 66 |
| ASFTTFTVT | 21 | KASFTTFTVT | 67 |
| KASFTTFTVTKY | 22 | FTTFTVTKYWF | 68 |
| IWKASFTTFTVT | 23 | SFTTFTVTKYW | 69 |
| SFTTFTVTKYWFY | 24 | ASFTTFTVTKY | 70 |
| KASFTTFTVTKYW | 25 | KASFTTFTVTK | 71 |
| IWKASFTTFTVTK | 26 | WKASFTTFTVT | 72 |
| FTTFTVTKYWFYRL | 27 | FTTFTVTKYWFY | 73 |
| ASFTTFTVTKYWFY | 28 | SFTTFTVTKYWF | 74 |
| WKASFTTFTVTKYW | 29 | ASFTTFTVTKYW | 75 |
| GIWKASFTTFTVTK | 30 | WKASFTTFTVTK | 76 |
| FTTFTVTKYWFYRLL | 31 | FTTFTVTKYWFYR | 77 |
| ASFTTFTVTKYWFYR | 32 | ASFTTFTVTKYWF | 78 |
| WKASFTTFTVTKYWF | 33 | WKASFTTFTVTKY | 79 |
| GIWKASFTTFTVTKY | 34 | GIWKASFTTFTVT | 80 |
| FDGIWKASFTTFTVT | 35 | SFTTFTVTKYWFYR | 81 |
| SFTTFTVTKYWFYRLL | 36 | KASFTTFTVTKYWF | 82 |
| KASFTTFTVTKYWFYR | 37 | IWKASFTTFTVTKY | 83 |
| IWKASFTTFTVTKYWF | 38 | DGIWKASFTTFTVT | 84 |
| DGIWKASFTTFTVTKY | 39 | SFTTFTVTKYWFYRL | 85 |
| SFDGIWKASFTTFTVT | 40 | KASFTTFTVTKYWFY | 86 |
| SFTTFTVTKYWFYRLLS | 41 | IWKASFTTFTVTKYW | 87 |
| KASFTTFTVTKYWFYRL | 42 | DGIWKASFTTFTVTK | 88 |
| IWKASFTTFTVTKYWFY | 43 | FTTFTVTKYWFYRLLS | 89 |
| DGIWKASFTTFTVTKYW | 44 | ASFTTFTVTKYWFYRL | 90 |
| SFDGIWKASFTTFTVTK | 45 | WKASFTTFTVTKYWFY | 91 |
| FTTFTVTKYWFYRLLSAL | 46 | GIWKASFTTFTVTKYW | 92 |
| ASFTTFTVTKYWFYRLLS | 47 | FDGIWKASFTTFTVTK | 93 |
| WKASFTTFTVTKYWFYRL | 48 | FTTFTVTKYWFYRLLSA | 94 |
| GIWKASFTTFTVTKYWFY | 49 | ASFTTFTVTKYWFYRLL | 95 |
| FDGIWKASFTTFTVTKYW | 50 | WKASFTTFTVTKYWFYR | 96 |
| HSFDGIWKASFTTFTVTK | 51 | GIWKASFTTFTVTKYWF | 97 |
| FTTFTVTKYWFYRLLSALF | 52 | FDGIWKASFTTFTVTKY | 98 |
| ASFTTFTVTKYWFYRLLSA | 53 | HSFDGIWKASFTTFTVT | 99 |
| WKASFTTFTVTKYWFYRLL | 54 | SFTTFTVTKYWFYRLLSA | 100 |
| GIWKASFTTFTVTKYWFYR | 55 | KASFTTFTVTKYWFYRLL | 101 |
| FDGIWKASFTTFTVTKYWF | 56 | IWKASFTTFTVTKYWFYR | 102 |
| HSFDGIWKASFTTFTVTKY | 57 | DGIWKASFTTFTVTKYWF | 103 |
| GTHSFDGIWKASFTTFTVT | 58 | SFDGIWKASFTTFTVTKY | 104 |
| FTTFTVTK | 59 | THSFDGIWKASFTTFTVT | 105 |
| SFTTFTVT | 60 | SFTTFTVTKYWFYRLLSAL | 106 |
| FTTFTVTKY | 61 | KASFTTFTVTKYWFYRLLS | 107 |
| SFTTFTVTK | 62 | IWKASFTTFTVTKYWFYRL | 108 |
| ASFTTFTVT | 63 | DGIWKASFTTFTVTKYWFY | 109 |
| FTTFTVTKYW | 64 | SFDGIWKASFTTFTVTKYW | 110 |
| SFTTFTVTKY | 65 | THSFDGIWKASFTTFTVTK | 111 |

In some embodiments, the Cav-1 peptide or the modified Cav-1 peptide:
a) consists of any one of the amino acid sequences of SEQ ID NOs: 2-111;
b) comprises a core sequence of any one of the amino acid sequences of SEQ ID NOs: 2-111; or
c) comprises a core sequence of any one of the amino acid sequences of SEQ ID NOs: 2-111, wherein the core sequence includes one or more amino acid substitutions, insertions, deletions, or chemical modifications.

In some embodiments, the Cav-1 peptide comprises or consists of the amino acid sequence of SEQ ID NO: 1. In some embodiments, the Cav-1 peptide comprises an amino acid sequence with at least 40%, at least 50%, at least 60%, at least 70%, at least 80%, at least 85%, at least 90%, at least 95%, at least 96%, at least 97%, at least 98%, or at least 99% sequence identity to SEQ ID NO: 1. In some embodiments, the Cav-1 peptide comprises the amino acid sequence of SEQ ID NO: 1 with one or more mutations relative thereto. For example, in some embodiments, the Cav-1 peptide comprises 1, 2, 3, 4, 5, 6, 7, 8, 9, 10, or more mutations relative to SEQ ID NO: 1. In some embodiments, the Cav-1 peptide comprises the amino acid sequence of SEQ ID NO: 1 with 1-5, 5-10, 11-5, 15-20, 10-25, 25-30, or more than 30 mutations. In some embodiments, the modified Cav-1 peptide comprises 1, 2, 3, 4, 5 or more amino acid substitutions, deletions, or insertions relative to the sequence of SEQ ID NO: 1, such as to derive a peptide of 3, 4, 5, 6, 7, 8, 9, 10, 11, 12, 13, 14, 15, 16, 17, 18, or 19 residues.

In some embodiments, the modified Cav-1 peptide comprises or consists of the amino acid sequence of any one of SEQ ID NOs: 2-111. In some embodiments, the modified Cav-1 peptide comprises an amino acid sequence with at least 40%, at least 50%, at least 60%, at least 70%, at least 80%, at least 85%, at least 90%, at least 95%, at least 96%, at least 97%, at least 98%, or at least 99% sequence identity to any one of SEQ ID NOs: 2-111. In some embodiments, the modified Cav-1 peptide comprises the amino acid sequence of any one of SEQ ID NOs: 2-111 with one or more mutations relative thereto. For example, in some embodiments, the modified Cav-1 peptide comprises 1, 2, 3, 4, 5, 6, 7, 8, 9, 10, or more mutations relative to any one of SEQ ID NOs: 2-111. In some embodiments, the modified Cav-1 peptide comprises the amino acid sequence of any one of SEQ ID NOs: 2-111 with 1-5, 5-10, or 11-15, or more mutations. In some embodiments, the modified Cav-1 peptide comprises an additional 1-5 amino acids at either the N- or C-terminus or at both termini of any one of SEQ ID NOs: 2-111.

In some embodiments, the modified Cav-1 peptide comprises or consists of the amino acid sequence of SEQ ID NOs: 2-10. In some embodiments, the modified Cav-1 peptide comprises an amino acid sequence with at least 40%, at least 50%, at least 60%, at least 70%, at least 80%, or at least 85% sequence identity to SEQ ID NO: 2-10. In some embodiments, the modified Cav-1 peptide comprises the amino acid sequence of SEQ ID NO: 3 with one or more mutations relative thereto. For example, in some embodiments, the modified Cav-1 peptide comprises 1, 2, 3, 4, or 5 mutations relative to SEQ ID NO: 2-10. In some embodiments, the modified Cav-1 peptide comprises an additional 1-5 amino acids at either the N- or C-terminus or at both termini of SEQ ID NO: 2-10. In some embodiments, the modified Cav-1 peptide of SEQ ID NO: 2-10 comprises an N- and/or C-terminal modification.

In some embodiments, the N-terminal modification is acylation. In some embodiments, the C-terminal modification is amidation.

In some embodiments, the polypeptide of the present disclosure consists of 20 or less amino acids. In some embodiments, polypeptide comprising an amino acid sequence of SEQ ID NO: 3 further comprises at least one amino acid added to the N-terminus; at least one amino acid added to the C-terminus; or at least one amino acid added to the N-terminus and the C-terminus. In these embodiments the addition made within 5 amino acids of each terminus has an amino acid sequence that is less than 90%, 80%, 70%, 60%, 50%, 40%, 30%, 20%, 10%, 0% (or any value or subranges therebetween) identical to a contiguous amino acid sequence of SEQ ID NO:1. In these embodiments the addition made within 5 amino acids of each terminus has an amino acid sequence that is less than 90%, 80%, 70%, 60%, 50%, 40%, 30%, 20%, 10%, 0% (or any value or subranges therebetween) identical to a contiguous amino acid sequence of SEQ ID NO:2. In these embodiments the addition made within 5 amino acids of each terminus has an amino acid sequence that is less than 80% identical to a contiguous amino acid sequence of SEQ ID NO:1 or 2. In some embodiments, the addition made within 5 amino acids of each terminus has an amino acid sequence that is less than 60% identical to a contiguous amino acid sequence of SEQ ID NO: 1 or 2. In some the addition made within 5 amino acids of each terminus has an amino acid sequence that is less than 40% identical to a contiguous amino acid sequence of SEQ ID NO: 1 or 2. In some embodiments, the addition made within 5 amino acids of each terminus has an amino acid sequence that is less than 20% identical to a contiguous amino acid sequence of SEQ ID NO: 1 or 2. In some embodiments, the polypeptide sequence comprises the peptide comprises L-amino acids; the peptide comprises D-amino acids; or the peptide comprises both L- and D-amino acids.

In some embodiments, the polypeptide of the present disclosure comprises at least one non-standard amino acid; or the polypeptide comprises two non-standard amino acids. In some embodiments, the non-standard amino acid is ornithine. In some embodiments, the polypeptide further comprises an N-terminal modification; a C-terminal modification; or an N- and C-terminal modification. In some embodiments, the N-terminal modification is acylation and/or the C-terminal modification is amidation.

In some embodiments, the polypeptide comprises a core sequence of ASFTTFTVT.

In some embodiments, the polypeptide comprises the amino acid sequence of FTTFTVT (SEQ ID NO: 3), KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10). In some embodiments, the polypeptide consists of an amino acid sequence selected from FTTFTVT (SEQ ID NO: 3), KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10). In some embodiments, polypeptide consists of an amino acid sequence of Ac-aaEGKASFTTFTVTKGSaa-NH2 (SEQ ID NO: 8).

In some embodiments, the modified Cav-1 peptide comprises 1, 2, 3, 4 or more amino acid substitutions, deletions, or insertions relative to the sequence of SEQ ID NO: 1, such as to derive a polypeptide of 3, 4, 5, 6, 7, 8, 9, 10, 11, 12, 13, 14, 15, 16, 17, 18, or 19 residues.

The modified Cav-1 peptides provided in the present disclosure exhibit similar or the same biological activity of the native Cav-1 peptide in in vitro or in vivo assays. In some embodiments, the modified Cav-1 peptide inhibits or prevents apoptosis of lung epithelial cells induced by bleomycin in vitro or in vivo with activity at least about 20% of the activity of the native Cav-1 peptide, or at least about 30%, 40%, 50%, 60%, 65%, 70%, 75%, 80%, 85%, 90%, about 95%, 97%, 99%, and any range derivable therein, such as, for example, from about 70% to about 80%, from about 81% to about 90%; or from about 91% to about 99%. The modified Cav-1 peptide may have 100% or even greater activity than the native Cav-1 peptide.

Assays for testing biological activity, e.g., anti-fibrotic activity, the ability to affect expression of uPA, uPAR and PAI-1 mRNAs, or inhibit proliferation of lung fibroblasts, are well-known in the art.

The modified Cav-1 peptides of the present disclosure are fragments, derivatives, or variants of the native Cav-1 peptide. The peptides can be synthetic, recombinant, or chemically modified peptides isolated or generated using methods well known in the art. Modifications can be made to amino acids on the N-terminus, C-terminus, or internally. Peptides can include conservative or non-conservative amino acid changes, as described below. Polynucleotide changes can result in amino acid substitutions, additions, deletions, fusions and truncations in the Cav-1 peptide encoded by the reference sequence. Peptides can also include insertions, deletions or substitutions of amino acids, including insertions and substitutions of amino acids (and other molecules) that do not normally occur in the peptide sequence that is the basis of the modified variant, for example but not limited to, insertion of L-amino acids, or non-standard amino acids such as ornithine, which do not normally occur in human proteins.

### A. Substitutions

In some embodiments, the modified Cav-1 peptide comprises one or more conservative amino acid substitutions. Conservative amino acid substitutions result from replacing one amino acid with another having similar structural and/or chemical properties, such as the replacement of a leucine with an isoleucine or valine, an aspartate with a glutamate, or a threonine with a serine. Thus, a conservative substitution of a particular amino acid sequence refers to substitution of those amino acids that are not critical for peptide activity or substitution of amino acids with other amino acids having similar properties (e.g., acidic, basic, positively or negatively charged, polar or non-polar, etc.) such that the substitution of even critical amino acids does not reduce the activity of the peptide. Conservative substitution tables providing functionally similar amino acids are well known in the art. For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). In some embodiments, individual substitutions, deletions, or additions that alter, add, or delete a single amino acid or a small percentage of amino acids can also be considered conservative substitutions if the change does not reduce the activity of the peptide. Insertions or deletions are typically in the range of about 1 to 6 amino acids.

In some embodiments, one can select the amino acid that will substitute an existing amino acid based on the location of the existing amino acid, i.e., its exposure to solvents (i.e., if the amino acid is exposed to solvents or is present on the outer surface of the peptide or peptide as compared to internally localized amino acids not exposed to solvents). Selection of such conservative amino acid substitutions are well known in the art, for example as disclosed in Dordo et al, J. Mol Biol, 1999, 217, 721-739 and Taylor et al, J. Theor. Biol. 119(1986); 205-218 and S. French and B. Robson, J Mol. Evol. 19(1983)171. Accordingly, one can select conservative amino acid substitutions suitable for amino acids on the exterior of a protein or peptide (i.e. amino acids exposed to a solvent), for example, but not limited to, the following substitutions can be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In some embodiments, one can select conservative amino acid substitutions suitable for amino acids on the interior of a protein or peptide, for example one can use suitable conservative substitutions for amino acids on the interior of a protein or peptide (i.e. the amino acids are not exposed to a solvent), for example but not limited to, one can use the following conservative substitutions: where Y is substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions are also encompassed within the term of variants.

In some embodiments, amino acid substitutions can be made in a peptide at one or more positions wherein the substitution is for an amino acid having a similar hydrophilicity. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus, such conservative substitutions can be made in a peptide and will likely only have minor effects on their activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4). These values can be used as a guide and thus, substitution of amino acids whose hydrophilicity values are within ±2 are preferred, those that are within ±1 are particularly preferred, and those within ±0.5 are even more particularly preferred. Thus, any of the Cav-1 peptides described herein may be modified by the substitution of an amino acid, for a different, but homologous amino acid with a similar hydrophilicity value. Amino acids with hydrophilicities within +/−1.0 points, or +/−0.5 points, are considered homologous.

In some embodiments, the modified Cav-1 peptide comprises non-naturally occurring amino acids. In some embodiments, the modified Cav-1 peptide comprises a combination of naturally occurring and non-naturally occurring amino acids, or comprises only non-naturally occurring amino acids. The non-naturally occurring amino acids can include synthetic non-native amino acids, substituted amino acids, or one or more D-amino acids (or other components of the formulation, with exception for protease recognition sequences) as desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes, and prolonged intravascular and interstitial lifetimes when such properties are desirable. Additionally, D-peptides cannot be processed efficiently for major histocompatibility complex class II-restricted presentation to T helper cells and are therefore less likely to induce humoral immune responses in the whole organism.

In addition to the 20 “standard” L-amino acids, D-amino acids or non-standard, modified or unusual amino acids, which are well-defined in the art, are also contemplated for use in the present disclosure. Phosphorylated amino acids (Ser, Thr, Tyr), glycosylated amino acids (Ser, Thr, Asn), β-amino acids, GABA, ω-amino acids are further contemplated for use in the present disclosure. These include, for example, include β-alanine (β-Ala) and other ω-amino acids such as β-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); norleucine (Nle); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); homoarginine (hArg); N-acetyl lysine (AcLys); 2,4-diaminobutyric acid (Dbu; Dab); p-aminophenylalanine (Phe(pNH₂)); N-methyl valine (MeVal); homocysteine (hCys), homophenylalanine (hPhe), and homoserine (hSer); hydroxyproline (Hyp), homoproline (hPro), N-methylated amino acids, and peptoids (N-substituted glycines).

A peptide or peptide region has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity can be determined using software programs known in the art, for example those described in Current Protocols In Molecular Biology (F. M. Ausubel et al., eds., 1987) Supplement 30, section 7.7.18, Table 7.7.1. Default parameters can be used for alignment.

### B. Derivatives

In some embodiments, the modified Cav-1 peptides are derivatives of the native Cav-1 peptide. The term “derivative” as used herein refers to Cav-1 peptides which have been chemically modified by using techniques including, but not limited to, acetylation, ubiquitination, labeling, pegylation (derivatization with polyethylene glycol), lipidation, glycosylation, amidation, cyclization, or addition of other molecules. In some embodiments, the peptide is be provided in a cyclic form, e.g., as a cyclic peptide or as a lactam. Alternatively, or in addition, in some embodiments, the peptide is provided as a branched peptide. A molecule is also a “derivative” of another molecule when it contains additional chemical moieties not normally a part of the molecule. Such moieties can alter the pH or improve the molecule's solubility, absorption, biological half-life, etc. The moieties can alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, PA (1990), incorporated herein, by reference, in its entirety.

The term “functional” when used in conjunction with “derivative” or “variant” refers to a peptide of the invention that possesses a biological activity (either functional or structural) that is substantially similar to a biological activity of the entity or molecule it is a functional derivative or functional variant thereof. The term functional derivative is intended to include the fragments, analogues or chemical derivatives of a molecule.

In some embodiments, the modified Cav-1 peptides may comprise co-translational and post-translational (e.g., C-terminal peptide cleavage) modifications, such as, for example, disulfide-bond formation, glycosylation, acetylation, phosphorylation, proteolytic cleavage (e.g., cleavage by furins or metalloproteases), and the like to the extent that such modifications do not affect the function of the modified Cav-1 peptides.

In some embodiments, the modified Cav-1 peptides can be “retro-inverso peptides.” A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are peptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

### C. Terminal Modifications

In some embodiments, the Cav-1 peptides of the present disclosure is modified (when linear) at its amino terminus or carboxy terminus. Examples of amino terminal modifications include, e.g., N-glycated, N-alkylated, N-acetylated or N-acylated amino acid. A terminal modification can include a pegylation. An example of a carboxy terminal modification is a C-terminal amidated amino acid. In some embodiments, the peptides are cross-linked or have a cross-linking site (for example, the modified Cav-1 peptide has a cysteinyl residue and thus forms cross-linked dimers in vitro or in vivo. In some embodiments, one or more peptidyl bonds are replaced by a non-peptidyl linkage; the N-terminus or the C-terminus is replaced, and individual amino acid moieties are modified through treatment with agents capable of reacting with selected side chains or terminal residues, and so forth. Either the C-terminus or the N-terminus of the amino acid sequences, or both, can be linked to a carboxylic acid functional group or an amine functional group, respectively. In some embodiments, the modified Cav-1 peptide comprises an N-terminal modification. In some embodiments, the modified Cav-1 peptide comprises a C-terminal modification. In some embodiments, the modified Cav-1 peptide comprises an N-terminal and a C-terminal modification.

Non-limiting, illustrative examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include, but are not limited to, acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl, phenyl-CO—, substituted phenyl-CO—, benzyl-CO— and (substituted benzyl)—CO—. Examples of alkoxy carbonyl and aryloxy carbonyl groups include, but are not limited to, CH₃—O-CO—, (ethyl)-O-CO—, n-propyl-O-CO—, iso-propyl-O-CO—, n-butyl-O-CO—, sec-butyl-O-CO—, t-butyl-O-CO, phenyl-O-CO—, (substituted phenyl)-O-CO—, benzyl-O-CO—, and (substituted benzyl)-OCO—. In order to facilitate the N-acylation, one to four glycine residues can be present at the N-terminus of the molecule.

Carboxy terminal modifications include acylation with carboxylic acids: formic acid, acetic acid, propionic acid, fatty acid (myristic, palmitic, stearic), succinic acid, and benzoic acid; carbonylation (such as benzyloxycarbonylation (Cbz)); acetylation; and biotinylation. Amino terminal modifications include, but are not limited to: (i) acylation with carboxylic acids: formic acid, acetic acid, propionic acid, fatty acid (myristic, palmitic, stearic, etc), succinic acid, benzoic acid; (ii) carbonylation (such as benzyloxycarbonylation (Cbz)); (iii) biotinylation; (iv) amidation; (v) attachment of dyes such as fluorescein (FITC, FAM, etc.), 7-hydroxy-4-methylcoumarin-3-acetic acid, 7-hydroxycoumarin-3-acetic acid, 7-metoxycoumarin-3-acetic acid and other coumarins; rhodamines (5-carboxyrhodamine 110 or 6G, 5(6)-TAMRA, ROX); N-[4-(4-dimethylamino)phenylazo]bezoic acid (Dabcyl), 2,4-dinitrobenzene (Dnp), 5-dimethylaminonaphthalene-1-sulfonic acid (Dansyl) and other dyes; and (vi) pegylation.

The carboxyl group at the C-terminus of a peptide can be protected, for example, by a group including, but not limited to, an amide (i.e., the hydroxyl group at the C-terminus is replaced with —NH2, —NHR2 and —NR2R3) or ester (i.e. the hydroxyl group at the C-terminus is replaced with —OR2). R2 and R3 are optionally independently an aliphatic, substituted aliphatic, benzyl, substituted benzyl, aryl or a substituted aryl group. In addition, taken together with the nitrogen atom, R2 and R3 can optionally form a C4 to C8 heterocyclic ring with from about 0-2 additional heteroatoms such as nitrogen, oxygen or sulfur. Non-limiting examples of heterocyclic rings include, but are not limited to, piperidinyl, pyrrolidinyl, morpholinyl, thiomorpholinyl or piperazinyl. Examples of C-terminal protecting groups include, but are not limited to, —NH₂, —NHCH₃, N(CH₃)₂, —NH(ethyl), N(ethyl)₂, N(methyl)(ethyl), —NH(benzyl), N(C₁-C₄ alkyl)(benzyl), —NH(phenyl), N(C₁-C₄ alkyl)(phenyl), OCH₃, —O-(ethyl), —O-(n-propyl), —O-(n-butyl), —O-(iso-propyl), —O-(sec-butyl), —O-(t-butyl), —O-benzyl and —O-phenyl.

### D. Side Chain Modifications

In some embodiments, the modified Cav-1 peptides of the present disclosure comprise a modified amino acid side chain. Non-limiting examples of modifications include carboxymethylation, acylation, phosphorylation, glycosylation, or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Gang and Jeanloz, Advances in Carbohydrate Chemistry and Biochemistry, Vol. 43, Academic Press (1985); Kunz, Ang. Chem. Int. Ed English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a modified Cav-1 peptide of the present disclosure, refers to a peptide wherein at least one of its amino acid residues is modified either by natural processes, such as processing or other post-translational modifications, or by chemical modification techniques which are well known in the art.

Examples of the numerous known modifications typically include, but are not limited to: acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference in its entirety. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference in its entirety. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

Covalent modifications of the modified Cav-1 peptides of the present disclosure are included within the scope of this invention. Other types of covalent modifications of the peptides are introduced into the molecule by reacting targeted amino acid residues with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide is also useful; the reaction, in some embodiments, is performed in 0.1M sodium cacodylate at pH 6.0.

Lysinyl and amino-terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin.

Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively. Tyrosyl residues are iodinated using 125 I or 1311 to prepare labeled peptides for use in radioimmunoassay.

Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl)carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking to a water-insoluble support matrix or surface for use in the method for purifying anti-CHF antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light.

Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group.

### E. Capping

In some embodiments, the modified Cav-1 peptide is capped at its N- and C-termini with an acyl (abbreviated “Ac”) and an amido (abbreviated “Am”) group, respectively, for example acetyl (CH₃CO—) at the N-terminus and amido (—NH₂) at the C-terminus. In some embodiments, the modified Cav-1 peptide is capped at its N-terminus with an acyl group, for example, an acetyl (CH₃CO—) at the N-terminus. In some embodiments, the modified Cav-1 peptide is capped at its C-terminus with an amido group, for example, an amido (—NH₂) at the C-terminus.

In some embodiments, the modified Cav-1 peptide is capped at its N-terminus. A broad range of N-terminal capping functions, such as a linkage to the terminal amino group, is contemplated, for example:
formyl;
alkanoyl, having from 1 to 10 carbon atoms, such as acetyl, propionyl, butyryl; alkenoyl, having from 1 to 10 carbon atoms, such as hex-3-enoyl;
alkynoyl, having from 1 to 10 carbon atoms, such as hex-5-ynoyl;
aroyl, such as benzoyl or 1-naphthoyl;
heteroaroyl, such as 3-pyrroyl or 4-quinoloyl;
alkylsulfonyl, such as methanesulfonyl;
arylsulfonyl, such as benzenesulfonyl or sulfanilyl;
heteroarylsulfonyl, such as pyridine-4-sulfonyl;
substituted alkanoyl, having from 1 to 10 carbon atoms, such as 4-aminobutyryl; substituted alkenoyl, having from 1 to 10 carbon atoms, such as 6-hydroxy-hex-3-enoyl;
substituted alkynoyl, having from 1 to 10 carbon atoms, such as 3-hydroxy-hex-5-ynoyl;
substituted aroyl, such as 4-chlorobenzoyl or 8-hydroxy-naphth-2-oyl;
substituted heteroaroyl, such as 2,4-dioxo-1,2,3,4-tetrahydro-3-methyl-quinazolin-6-oyl;
substituted alkylsulfonyl, such as 2-aminoethanesulfonyl;
substituted arylsulfonyl, such as 5-dimethylamino-1-naphthalenesulfonyl;
substituted heteroarylsulfonyl, such as 1-methoxy-6-isoquinolinesulfonyl;
carbamoyl or thiocarbamoyl;
substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl;
substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

In some embodiments, the modified Cav-1 peptide is capped at its C-terminus. The C-terminal capping function can either be in an amide or ester bond with the terminal carboxyl. Capping functions that provide for an amide bond are designated as NR¹R² wherein R¹ and R² may be independently drawn from the following group: hydrogen;
alkyl, such as having from 1 to 10 carbon atoms, such as methyl, ethyl, isopropyl; alkenyl, such as having from 1 to 10 carbon atoms, such as prop-2-enyl;
alkynyl, such as having from 1 to 10 carbon atoms, such as prop-2-ynyl;
substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl;
substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl;
substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;
aroylalkyl having up to 10 carbon atoms, such as phenacyl or 2-benzoylethyl;
aryl, such as phenyl or 1-naphthyl;
heteroaryl, such as 4-quinolyl;
alkanoyl having from 1 to 10 carbon atoms, such as acetyl or butyryl;
aroyl, such as benzoyl;
heteroaroyl, such as 3-quinoloyl; OR′ or NR′R″ where R′ and R″ are independently hydrogen, alkyl, aryl, heteroaryl, acyl, aroyl, sulfonyl, sulfinyl, or SO₂—R″′ or SO—R″′ where R″′ is substituted or unsubstituted alkyl, aryl, heteroaryl, alkenyl, or alkynyl.

Capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

In some embodiments, the N-terminal or the C-terminal capping function, or both, is of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups allows the addition of other activities on the peptide. For example, the presence of a sulfhydryl group linked to the N- or C-terminal cap will permit conjugation of the derivatized peptide to other molecules.

### F. Multimerization

Embodiments of the present disclosure also include longer peptides built from repeating units of a modified Cav-1 peptide. In some embodiments, a peptide multimer comprises different combinations of peptide. In some embodiments, multimeric peptides are made by chemical synthesis or by recombinant DNA techniques as discussed herein. When produced by chemical synthesis, the oligomers, in some embodiments, have from 2-5 repeats of a core peptide sequence, and the total number of amino acids in the multimer should not exceed about 160 residues, or not more than 100 residues (or their equivalents, when including linkers or spacers).

In some embodiments, the modified Cav-1 peptide is a multimer comprising at least two peptides of the present disclosure. In some embodiments, a first peptide of the at least two peptides is essentially identical to a second peptide of the at least two peptides. In some embodiments, a first peptide of the at least two peptides is not identical to a second peptide of the at least two peptides.

### G. Peptidomimetics

In some embodiments, the modified Cav-1 peptide is a peptidomimetic compound which mimics the biological effects of the native Cav-1 peptide. In some embodiments, the peptidomimetic agent is an unnatural peptide or a non-peptide agent that recreates the stereospatial properties of the binding elements of the native Cav-1 peptide such that it has the binding activity and biological activity of the native Cav-1 peptide. Similar to a native Cav-1 peptide or peptide multimer, a peptidomimetic will have a binding face (which interacts with any ligand to which the native Cav-1 peptide binds) and a non-binding face.

In some embodiments, the present disclosure also includes modified Cav-1 peptides that retain partial peptide characteristics. For example, any proteolytically unstable bond within a Cav-1 peptide of the invention could be selectively replaced by a non-peptidic element such as an isostere (N-methylation; D-amino acid) or a reduced peptide bond while the rest of the molecule retains its peptidic nature.

Peptidomimetic compounds, either agonists, substrates or inhibitors, have been described for a number of bioactive peptides/peptides such as opioid peptides, VIP, thrombin, HIV protease, etc. Methods for designing and preparing peptidomimetic compounds are known in the art (Hruby, V J, Biopolymers 33:1073-1082 (1993); Wiley, R A et al., Med Res. Rev. 13:327-384 (1993); Moore et al., Adv. in Pharmacol 33:91-141 (1995); Giannis et al., Adv. in Drug Res. 29:1-78 (1997). Certain mimetics that mimic secondary structure are described in Johnson et al., In: Biotechnology and Pharmacy, Pezzuto et al., Chapman and Hall (Eds.), NY, 1993. These methods are used to make peptidomimetics that possess at least the binding capacity and specificity of the native Cav-1 peptide and, in some embodiments, also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the three-dimensional structure of a peptide of the invention either free or bound in complex with a ligand (e.g., soluble uPAR or a fragment thereof). Alternatively, the structure of a peptide of the invention bound to its ligand can be gained by the techniques of nuclear magnetic resonance spectroscopy. Greater knowledge of the stereochemistry of the interaction of the peptide with its ligand or receptor will permit the rational design of such peptidomimetic agents. The structure of a peptide or peptide of the invention in the absence of ligand could also provide a scaffold for the design of mimetic molecules.

### H. PEGylation

In some embodiments, the modified Cav-1 peptides of the present disclosure are conjugated with heterologous peptide segments or polymers, such as polyethylene glycol. In some embodiments, the modified Cav-1 peptides are linked to PEG to increase the hydrodynamic radius of the enzyme and hence increase the serum persistence. In some embodiments, the modified Cav-1 peptides are conjugated to any targeting agent, such as a ligand having the ability to specifically and stably bind to an external receptor (see e.g., U.S. Patent Publ. No. 2009/0304666).

In some embodiments, the present disclosure provides methods and formulations related to PEGylation of Cav-1 peptides. PEGylation is the process of covalent attachment of poly(ethylene glycol) polymer chains to another molecule, normally a drug or therapeutic protein. PEGylation is routinely achieved by incubation of a reactive derivative of PEG with the target macromolecule. The covalent attachment of PEG to a drug or therapeutic protein can “mask” the agent from the host's immune system (reduced immunogenicity and antigenicity) or increase the hydrodynamic size (size in solution) of the agent, which prolongs its circulatory time by reducing renal clearance. PEGylation can also provide water solubility to hydrophobic drugs and proteins.

The first step of PEGylation is the suitable functionalization of the PEG polymer at one or both terminals. PEGs that are activated at each terminus with the same reactive moiety are known as “homobifunctional,” whereas if the functional groups present are different, then the PEG derivative is referred as “heterobifunctional” or “heterofunctional.” The chemically active or activated derivatives of the PEG polymer are prepared to attach the PEG to the desired molecule.

The choice of the suitable functional group for the PEG derivative is based on the type of available reactive group on the modified Cav-1 peptide that will be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid group can also be used.

The techniques used to form first generation PEG derivatives are generally reacting the PEG polymer with a group that is reactive with hydroxyl groups, typically anhydrides, acid chlorides, chloroformates, and carbonates. In the second generation PEGylation chemistry more efficient functional groups, such as aldehyde, esters, amides, etc., are made available for conjugation.

As applications of PEGylation have become more and more advanced and sophisticated, there has been an increased need for heterobifunctional PEGs for conjugation.

These heterobifunctional PEGs are very useful in linking two entities, where a hydrophilic, flexible, and biocompatible spacer is needed. Preferred end groups for heterobifunctional PEGs are maleimide, vinyl sulfones, pyridyl disulfide, amine, carboxylic acids, and N-hydroxysuccinimide (NHS)esters.

The most common modification agents, or linkers, are based on methoxy PEG (mPEG) molecules. Their activity depends on adding a protein-modifying group to the alcohol end. In some embodiments, polyethylene glycol (PEG diol) is used as the precursor molecule. The diol is subsequently modified at both ends in order to make a hetero- or homo-dimeric PEG-linked molecule.

Proteins are generally PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four are strongly cysteinyl-specific under mild conditions and neutral to slightly alkaline pH but each has some drawbacks. The thioether formed with the maleimides can be somewhat unstable under alkaline conditions so there may be some limitation to formulation options with this linker. The carbamothioate linkage formed with iodo PEGs is more stable, but free iodine can modify tyrosine residues under some conditions. PEG thiols form disulfide bonds with protein thiols, but this linkage can also be unstable under alkaline conditions. PEG-vinylsulfone reactivity is relatively slow compared to maleimide and iodo PEG; however, the thioether linkage formed is quite stable. Its slower reaction rate also can make the PEG-vinylsulfone reaction easier to control.

Site-specific PEGylation at native cysteinyl residues is seldom carried out, since these residues are usually in the form of disulfide bonds or are required for biological activity. On the other hand, site-directed mutagenesis can be used to incorporate cysteinyl PEGylation sites for thiol-specific linkers. The cysteine mutation must be designed such that it is accessible to the PEGylation reagent and is still biologically active after PEGylation.

Amine-specific modification agents include PEG NHS ester, PEG tresylate, PEG aldehyde, PEG isothiocyanate, and several others. All react under mild conditions and are very specific for amino groups. The PEG NHS ester is probably one of the more reactive agents; however, its high reactivity can make the PEGylation reaction difficult to control on a large scale. PEG aldehyde forms an imine with the amino group, which is then reduced to a secondary amine with sodium cyanoborohydride. Unlike sodium borohydride, sodium cyanoborohydride will not reduce disulfide bonds. However, this chemical is highly toxic and must be handled cautiously, particularly at lower pH where it becomes volatile.

Due to the multiple lysine residues on most proteins, site-specific PEGylation can be a challenge. Because these reagents react with unprotonated amino groups, it is possible to direct the PEGylation to lower-pK amino groups by performing the reaction at a lower pH. Generally, the pK of the alpha-amino group is 1-2 pH units lower than the epsilon-amino group of lysine residues. By PEGylating the molecule at pH 7 or below, high selectivity for the N-terminus frequently can be attained. However, this is only feasible if the N-terminal portion of the protein is not required for biological activity. Still, the pharmacokinetic benefits from PEGylation frequently outweigh a significant loss of in vitro bioactivity, resulting in a product with much greater in vivo bioactivity regardless of PEGylation chemistry.

There are several parameters to consider when developing a PEGylation procedure. Fortunately, there are usually no more than four or five key parameters. The “design of experiments” approach to optimization of PEGylation conditions can be very useful. For thiol-specific PEGylation reactions, parameters to consider include: protein concentration, PEG-to-protein ratio (on a molar basis), temperature, pH, reaction time, and in some instances, the exclusion of oxygen. (Oxygen can contribute to intermolecular disulfide formation by the protein, which will reduce the yield of the PEGylated product.) The same factors should be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker should be known before starting the PEGylation reaction. For example, if the PEGylation agent is only 70 percent active, the amount of PEG used should ensure that only active PEG molecules are counted in the protein-to-PEG reaction stoichiometry.

### I. Fusion Proteins

In some embodiments, the present disclosure provides fusion proteins of the modified Cav-1 peptides. For example, fusions may employ leader sequences from other species to permit the recombinant expression of a protein in a heterologous host. Fusion proteins can comprise a half-life extender. Another useful fusion includes the addition of a protein affinity tag, such as a serum albumin affinity tag or six histidine residues, or an immunologically active domain, such as an antibody epitope, including those that are cleavable, to facilitate purification of the fusion protein. Non-limiting affinity tags include polyhistidine, chitin binding protein (CBP), maltose binding protein (MBP), and glutathione-S-transferase (GST). In some embodiments, the modified Cav-1 peptide comprises a heterologous peptide or protein linked at the N- and/or C-terminus. In some embodiments, the heterologous peptide or protein is a leader sequence, a half-life extender, a protein affinity tag, or an immunologically active domain.

In some embodiments, the modified Cav-1 peptide is linked to a peptide that increases the in vivo half-life, such as an XTEN® peptide (Schellenberger et al., 2009), IgG Fc domain, albumin, or albumin binding peptide.

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the peptides connected in tandem. The linker would be of sufficient length to allow proper folding of the resulting fusion protein.

### i) Linkers

In some embodiments, the modified Cav-1 peptide is chemically conjugated using bifunctional cross-linking reagents or fused at the protein level with peptide linkers.

Bifunctional cross-linking reagents have been extensively used for a variety of purposes, including preparation of affinity matrices, modification and stabilization of diverse structures, identification of ligand and receptor binding sites, and structural studies. In some embodiments, peptide linkers such as Gly-Ser linkers are used to link the modified Cav-1 peptides of the present disclosure.

Homobifunctional reagents that carry two identical functional groups proved to be highly efficient in inducing cross-linking between identical and different macromolecules or subunits of a macromolecule, and linking of peptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidine-, indole-, carboxyl-specific groups. Of these, reagents directed to free amino groups have become especially popular because of their commercial availability, ease of synthesis, and the mild reaction conditions under which they can be applied.

A majority of heterobifunctional cross-linking reagents contain a primary amine-reactive group and a thiol-reactive group. In another example, heterobifunctional cross-linking reagents and methods of using the cross-linking reagents are described (U.S. Pat. No. 5,889,155, incorporated herein by reference in its entirety). The cross-linking reagents combine a nucleophilic hydrazide residue with an electrophilic maleimide residue, allowing coupling, in one example, of aldehydes to free thiols. The cross-linking reagent can be modified to cross-link various functional groups.

Additionally, any other linking/coupling agents and/or mechanisms known to those of skill in the art may be used to combine the modified Cav-1 peptides of the present disclosure, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

In some embodiments, the modified Cav-1 peptide comprises a cross-linker that has reasonable stability in the blood. Numerous types of disulfide-bond containing linkers are known that can be successfully employed to conjugate targeting and therapeutic/preventative agents. Linkers that contain a disulfide bond that is sterically hindered may prove to give greater stability in vivo. Thus, in some embodiments, the modified Cav-1 peptide comprises a sterically hindered cross-linker.

In addition to hindered cross-linkers, non-hindered linkers also can be employed in accordance herewith. In some embodiments, the modified Cav-1 peptide comprises a non-sterically hindered cross linker. Other useful cross-linkers, not considered to contain or generate a protected disulfide, include SATA, SPDP, and 2-iminothiolane (Wawrzynczak and Thorpe, 1987). The use of such cross-linkers is well understood in the art.

In some embodiments, the modified Cav-1 peptide comprises a flexible linker.

Once chemically conjugated, the modified Cav-1 peptide generally will be purified to separate the conjugate from unconjugated agents and from other contaminants. A large number of purification techniques are available for use in providing conjugates of a sufficient degree of purity to render them clinically useful.

Purification methods based upon size separation, such as gel filtration, gel permeation, or high performance liquid chromatography, will generally be of most use. Other chromatographic techniques, such as Blue-Sepharose separation, may also be used.

Conventional methods to purify the fusion proteins from inclusion bodies may be useful, such as using weak detergents, such as sodium N-lauroyl-sarcosine (SLS).

### ii) Cell Penetrating and Membrane Translocation Peptides

In some embodiments, the modified Cav-1 peptides comprises a cell-binding domain or cell penetrating peptide (CPP). As used herein, the terms “cell penetrating peptide”, “membrane translocation domain”, and “protein transduction domain” are used interchangeably and refer to segments of a peptide sequence that allow a peptide to cross the cell membrane (e.g., the plasma membrane in the case a eukaryotic cell). Examples of CPPs include, but are not limited to, segments derived from HIV-binding peptides, HIV-1 Tat (HIV), Tat-derived peptides, Penetratin, VP22 derived or analog peptides, HSV VP22 (Herpes simplex), protegrin I, MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides (particularly from Drosophila Antennapedia), pAntp, T1 (TKIESLKEHG, SEQ ID NO: 115), T2 (TQIENLKEKG, SEQ ID NO: 116), 26 (AALEALAEALEALAEALEALAEAAAA, SEQ ID NO: 117), INF7 (GLFEAIEGFIENGWEGMIEGWYGCG, SEQ ID NO: 118) pIsl, FGF, Lactoferrin, Transportan, Buforin-2, Bac715-24, SynB, SynB(1), pVEC, hCT-derived peptides, SAP, or histones.

CPPs typically have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. Typically, CPPs are peptides of 8 to 50 residues that have the ability to cross the cell membrane and enter into most cell types. Frankel and Pabo described the ability of the trans-activating transcriptional activator from the human immunodeficiency virus 1 (HIV-TAT) to penetrate into cells (Frankel, A.D. and C.O. Pabo, Cellular uptake of the tat protein from human immunodeficiency virus. Cell, 1988. 55(6): p. 1189-93). In 1991, transduction into neural cells of the Antennapedia homeodomain (DNA-binding domain) from Drosophila melanogaster was also described (Joliot, A., et al., Antennapedia homeobox peptide regulates neural morphogenesis. Proc Natl Acad Sci USA, 1991. 88(5): p. 1864-8). In 1994, the first 16-mer peptide CPP called Penetratin (RQIKIWFQNRRMKWKK, SEQ ID NO: 113) was characterized from the third helix of the homeodomain of Drosophila Antennapedia homeobox gene product (Derossi, D., et al., The third helix of the Antennapedia homeodomain translocates through biological membranes. J Biol Chem, 1994. 269(14): p. 10444-50), followed in 1998 by the identification of the minimal domain of TAT required for protein transduction (e.g., GRKKRRQRRRPPQ, SEQ ID NO: 112) (Vives, E., P. Brodin, and B. Lebleu, A truncated HIV-1 Tat protein basic domain rapidly translocates through the plasma membrane and accumulates in the cell nucleus. J Biol Chem, 1997. 272(25): p. 16010-7). Over the past two decades, dozens of peptides were described from different origins including viral proteins, e.g., herpes virus VP22 (Elliott, G. and P. O'Hare, Intercellular trafficking and protein delivery by a herpesvirus structural protein. Cell, 1997. 88(2): p. 223-33), or from venoms, e.g. melittin (GIGAVLKVLTTGLPALISWIKRKRQQ, SEQ ID NO: 114) (Dempsey, C.E., The actions of melittin on membranes. Biochim Biophys Acta, 1990. 1031 (2): p. 143-61), mastoporan (Konno, K., et ah, Structure and biological activities of eumenine mastoparan-AF (EMP-AF), a new mast cell degranulating peptide in the venom of the solitary wasp (Anterhynchium flavomarginatum micado). Toxicon, 2000. 38(11):1505-15), maurocalcin (Esteve, E., et al., Transduction of the scorpion toxin maurocalcine into cells. Evidence that the toxin crosses the plasma membrane. J Biol Chem, 2005. 280(13): p. 12833-9), crotamine (Nascimento, F. D., et al., Crotamine mediates gene delivery into cells through the binding to heparan sulfate proteoglycans. J Biol Chem, 2007. 282(29): p. 21 349-60) or buforin (Kobayashi, S., et al., Membrane translocation mechanism of the antimicrobial peptide buforin 2. Biochemistry, 2004. 43(49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276(8): p. 5836-40) or transportan (Pooga, M., et al., Cell penetration by transportan. FASEB J, 1998. 12(1): p. 67-77). Any of the above described CPPs may be used in the modified Cav-1 peptides of the present disclosure. A number of other CPPs described in Milletti F. (Drug Discov Today 17 (15-16): 850-60, 2012), can also be used in the modified Cav-1 peptides of the present disclosure.

### III. Pharmaceutical Formulations

In some embodiments, the present disclosure relates to pharmaceutical formulations comprising modified Cav-1 peptides. In some embodiments, the pharmaceutical formulations as described herein can be useful in the treatment or prevention of a disease, an injury, or an infection as described herein.

In some embodiments, the pharmaceutical formulations comprising modified Cav-1 peptides are formulated to be administered intravenously, intrathecally, intradermally, transdermally, intrathecally, intraarterially, intraperitoneally, intranasally, intravaginally, intravesicular, intraarticular, intralesional, intrarectally, intramuscularly, subcutaneously, mucosally, orally, topically, locally, by inhalation (e.g., inhalation of a nebulized or dry powder formulation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). In some embodiments, the pharmaceutical formulations are formulated to be administered intravenously, intrathecally, subcutaneously and/or intraperitoneally.

In one aspect, the present disclosure provides pharmaceutical formulations comprising a modified Cav-1 peptide that comprises a core sequence of FTTFTVT and a sucrose acetate isobutyrate (SAIB). In some embodiments, the pharmaceutical formulations comprise a modified Cav-1 peptide that comprises a core sequence of ASFTTFTVT and SAIB. In some embodiments, SAIB is a mixed ester of sucrose esterified with two acetate and six isobutyrate groups where the ester is completely non-crystalline and has a viscosity of over 100,000 cP at 30° C.

In some embodiments, the formulation comprising the modified Cav-1 peptide is selected from any one of the polypeptides disclosed herein. In some embodiments, the formulation comprises SAIB.

In some embodiments, the SAIB is mixed with the modified Cav-1 peptide to prepare a suspension. In some embodiments, the SAIB and the modified Cav-1 peptide is mixed at a temperature between room temperature and about 37° C. In some embodiments, the SAIB and the modified Cav-1 peptide is mixed at a temperature at or below about 37° C. In some embodiments, the SAIB and the modified Cav-1 peptide can be mixed with one or more biocompatible solvents. In some embodiments, the SAIB and the modified Cav-1 peptide can be mixed with one or more biocompatible solvents selected from water, ethanol, isopropyl alcohol, saline, or phosphate buffer solution (PBS). In some embodiments, use of different solvents can alter the viscosity of the SAMB and Cav-1 peptide solution. In some embodiments, the modified Cav-1 peptide formulation is an injectable formulation.

In some embodiments, the SAIB formulation further comprises a first solvent selected from acetone, benzyl alcohol, butylene glycol, caprolactam, caprolactone, dimethylsulfoxide, ethanol, ethyl acetate, ethyl lactate, glycerol, glycerol formal, glycofurol, tetraglycol, N-methyl-2-pyrrolidone, polyethylene glycol, methoxy polyethylene glycol, alkoxy polyethylene glycol, propylene carbonate, 2 pyrrolidone, triacetin, triethyl citrate, or a combination thereof. In some embodiments, the first solvent is N-methylpyrrolidone (NMP), anhydrous ethanol, and/or ethyl acetate. In some embodiments, the first solvent is NMP and/or ethyl acetate.

In some embodiments, the SAIB is present in the first solvent in about 50% w/w to about 95% w/w, including any values or subranges therebetween. In some embodiments, the SAIB is present in the first solvent in about 50% w/w, about 51% w/w, about 52% w/w, about 53% w/w, about 54% w/w, about 55% w/w, about 56% w/w, about 57% w/w, about 58% w/w, about 59% w/w, about 60% w/w, about 61% w/w, about 62% w/w, about 63% w/w, about 64% w/w, about 65% w/w, about 66% w/w, about 67% w/w, about 68% w/w, about 69% w/w, about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, 82% w/w, about 83% w/w, about 84% w/w, about 85% w/w, about 86% w/w, about 87% w/w, 88% w/w, about 89% w/w, about 90% w/w, about 91% w/w, about 92% w/w, about 93% w/w, about 94% w/w, or about 95% w/w. In some embodiments, the SAIB is present in the first solvent in about 70% w/w to about 90% w/w, including any values or subranges therebetween. In some embodiments, the SAIB is present in the first solvent in about 80% w/w.

In some embodiments, the modified Cav-1 peptide of the present disclosure is micronized. In some embodiments, the modified Cav-1 peptide is micronized before admixing with SAIB. In some embodiments, the modified Cav-1 peptide is micronized during the formulation process. In some embodiments, the modified Cav-1 peptide particles are reduced in size by using techniques including but not limited to, grinding, milling (e.g., air-attrition milling (jet milling), ball milling), coacervation, complex coacervation, high pressure homogenization, spray drying and/or supercritical fluid crystallization. In some embodiments, the modified Cav-1 peptide particles are reduced in size by mechanical impact (e.g., by hammer mills, ball mill and/or pin mills). In some embodiments, the modified Cav-1 peptide is micronized via fluid energy (e.g., by spiral jet mills, loop jet mills, and/or fluidized bed jet mills). In some embodiments, the modified Cav-1 peptide particles are reduced in size by spray drying. In some embodiments, the modified Cav-1 peptide is micronized by spray drying.

In some embodiments, the formulation described herein comprises one or more multiparticulate modified Cav-1 peptides. In some embodiments, the formulation described herein comprises micronized modified Cav-1 peptides. In some embodiments, the micronized polypeptide has a mean particle size in the range of about 0.5 μm to about 500 μm, including any values or subranges therebetween. In some embodiments, the micronized polypeptide has a mean particle size in the range of about 0.5 μm to about 200 μm, including any values or subranges therebetween. In some embodiments, the micronized polypeptide has a mean particle size in the range of about 0.5 μm to about 100 μm, including any values or subranges therebetween. In some embodiments, the micronized polypeptide has a mean particle size in the range of about 1 μm to about 50 μm, including any values or subranges therebetween. In some embodiments, the micronized polypeptide has a mean particle size in the range of about 1 μm to about 5 μm, including any values or subranges therebetween. In some embodiments, the micronized polypeptide has a mean particle size of less than about 10 μm, less than about 5 μm, or less than about 3 μm. In some embodiments, the micronized peptide has a mean particle size of about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7, about 2.8 μm, about 2.9 μm, about 3.0 μm, about 3.1 μm, about 3.2 μm, about 3.3 μm, about 3.4 μm, about 3.5 μm, about 3.6 μm, about 3.7 μm, about 3.8 μm, about 3.9 μm, about 4.0 μm, about 4.1 μm, about 4.2 μm, about 4.3 μm, about 4.4 μm, about 4.5 μm, about 4.6 μm, about 4.7 μm, about 4.8 μm, about 4.9 μm, or about 5.0 μm.

In some embodiments, SAIB and the modified Cav-1 peptide can be combined by any suitable means known in the art, such as direct mixing, extrusion coating, spray drying, blending, and encapsulation. In some embodiments, SAIB and the modified Cav-1 peptide are combined and formulated into a pharmaceutical formulation as described in Example 1.

In some embodiments, the formulation is in the form of an emulsion, a solution, microspheres, nanoparticles, nanospheres, implants, or gels. In some embodiments, the formulation is a suspension. In some embodiments, the formulation further comprises a second solvent. In some embodiments, the second solvent is sterile water, phosphate-buffered saline (PBS), or any pharmaceutically acceptable diluent known in the art. In some embodiments, the second solvent is PBS. In some embodiments, the SAIB formulation is in the form of an emulsion, a solution, microspheres, nanoparticles, nanospheres, implants, or gels.

In one aspect, the present disclosure provides pharmaceutical formulations comprising a modified Cav-1 peptide that comprises a core sequence of FTTFTVT and a biodegradable polymer.

In some embodiments, the formulation comprising the modified Cav-1 peptides described herein is a parenteral depot formulation or in situ forming parenteral system (ISFI).

In some embodiments, the formulation comprises a mixture of modified Cav-1 peptide and biodegradable polymer(s) dissolved or suspended in a pharmaceutically acceptable water-miscible organic solvent. ISFI formulations and methods are further described in Musmade et al., J. Biol. Chem. Chron., 2019, 5(1), 14-21; Ko et al., Progress in Polymer Science, Vol. 38, 2013; and Kanwar and Sinha, Crit. Rev. Ther. Drug Carrier Systems, Vol. 36, 2019, which are herein incorporated by reference in their entirety.

In some embodiments, the formulation comprising the modified Cav-1 peptides described herein comprises a biodegradable polymer. In some embodiments, the biodegradable polymer is poly(lactic acid), poly(glycolic acid), poly(lactic-co-glycolic acid), poly(ethylene glycol), poly(vinyl alcohol), polysiloxane, poly(ethylene-vinyl acetate), polyurethane, polyalkylcyanoacrylate, poly-ε-caprolactone, or a hydrogel polymer.

In some embodiments, the formulation comprises the modified Cav-1 peptide and SAIB at a weight ratio of about 1:50 to about 1:1, about 1:50 to about 1:5, about 1:50 to about 1:10, or any value or subrange therebetween. In some embodiments, the modified Cav-1 peptide and the SAIB weight ratio is about 1:40 to about 1:1 or about 1:40 to about 1:5, including any values or subranges therebetween. In some embodiments, the modified Cav-1 peptide and the SAIB weight ratio is about 1:30 to about 1:1 or 1:30 to about 1:5, including any values or subranges therebetween. In some embodiments, the modified Cav-1 peptide and the SAIB weight ratio is about 1:20 to about 1:1 or about 1:20 to about 1:5, including any values or subranges therebetween. In some embodiments, the modified Cav-1 peptide and the SAIB weight ratio is about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4, about 1:3, about 1:2, about 1:1, or any value or subrange therebetween. In some embodiments, the modified Cav-1 peptide and the SAIB weight ratio is about 1:50. In some embodiments, the modified Cav-1 peptide and the SAIB weight ratio is about 1:40. In some embodiments, the modified Cav-1 peptide and the SAIB weight ratio is about 1:30. In some embodiments, the modified Cav-1 peptide and the SAIB weight ratio is about 1:17. In some embodiments, the modified Cav-1 peptide and the SAIB weight ratio is about 1:13. In some embodiments, the modified Cav-1 peptide and the SAIB weight ratio is about 1:10. In some embodiments, the modified Cav-1 peptide and the SAIB weight ratio is about 1:8. In some embodiments, the modified Cav-1 peptide and the SAIB weight ratio is about 1:5. In some embodiments, the modified Cav-1 peptide and the SAIB weight ratio is about 1:1.

In some embodiments, the formulation further includes any molecule or material that serves to minimize the chemical degradation of the modified Cav-1 peptide and/or maintains the physio-chemical stability of the formulation. Exemplary molecule or material includes amphiphilic molecules such as mono-C₁₂-1s alkyl sulfate sodium salts, dialkyl ester sulfosuccinic acid derivatives having 3 to 16 carbon atoms, dioctyl sulfosuccinic acid, benzenesulfonic acid, naphthalene-1,5-disulfonic acid, camphorsulfonic acid, (+)-(1S)-camphor-10-sulfonic acid, dodecylsulfuric acid, para-toluenesulfonic acid, naphthalene-2-sulfonic acid, cholesterol sulfate, heptanesulfonic acid, capric acid, caproic acid, caprylic acid, cinnamic acid, oleic acid, palmitic acid, pamoic acid, benzoic acid, stearic acid, undecylenic acid, and phospholipids; other polymers such as olylactides, polyglycolides, polycaprolactones, polyanhydrides, polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, polyhydroxycellulose, chitin, chitosan, hyaluronic acid and copolymers, terpolymers and mixtures thereof; and free radical scavengers and stabilizers such as cysteine or methionine, d-alpha tocopherol acetate, dl-alpha tocopherol, ascorbyl palmitate, butylated hydroxyanidole, butylated hydroxyanisole, butylatedhydroxyquinone, butylhydroxyanisol, hydroxycomarin, butylated hydroxytoluene, cephalm, ethyl gallate, propyl gallate, octyl gallate, lauryl gallate, propylhydroxybenzoate, trihydroxybutyrophenone, dimethylphenol, ditertbutylphenol, vitamin E, and lecithin. In some embodiments, the formulation comprises SAIB.

In some embodiments, the formulation further comprises a pharmaceutically acceptable carrier or excipient. In some embodiments, the formulation comprises SAIB.

In some embodiments, the formulation releases at least about 1% to about 20% of the modified Cav-1 peptide by four hours under physiological conditions. In some embodiments, the formulation releases at least about 1% to about 20% of the modified Cav-1 peptide by four hours when tested in a dissolution apparatus at a neutral pH. In some embodiments, the formulation releases at least about 1% to about 20% of the modified Cav-1 peptide by four hours when tested in a dissolution apparatus at about 37° C. In some embodiments, the formulation releases at least about 2% to about 15%, including any values or subranges therebetween, by four hours under physiological conditions. In some embodiments, the formulation releases at least about 2%, about 3%, about 4%, about 5%, about 6%, about 7%, about 8%, about 9%, about 10%, about 11%, about 12%, about 13%, about 14%, or about 15% of the peptide by four hours under physiological conditions. In some embodiments, the physiological condition is in an aqueous buffer in the range of about pH 6 to about pH 8 at about 37° C. In some embodiments, the physiological condition is in an aqueous buffer of about pH 7.4 at about 37° C. In some embodiments, the formulation comprises SAIB.

In some embodiments, the formulation releases the modified Cav-1 peptides under physiological conditions for at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days, or any value therein. In some embodiments, the formulation releases the modified Cav-1 peptides under physiological conditions for at least 1 week, 2 weeks, 3 weeks, 4 weeks, 5 weeks, 6 weeks, 7 weeks, 8 weeks, 9 weeks, 10 weeks, 11 weeks, or 12 weeks, or any value or a range therebetween. In some embodiments, the physiological condition is in an aqueous buffer in the range of about pH 6 to about pH 8 at about 37° C. In some embodiments, the physiological condition is in an aqueous buffer of about pH 7.4 at about 37° C.

In some embodiments, the formulation as described herein releases the modified Cav-1 peptides for at least eight hours under physiological conditions. In some embodiments, the formulation as described herein releases the modified Cav-1 peptides for at least twelve hours under physiological conditions. In some embodiments, the formulation as described herein releases the modified Cav-1 peptides for at least a day under physiological conditions. In some embodiments, the formulation as described herein releases the modified Cav-1 peptides for at least 2 days under physiological conditions. In some embodiments, the formulation releases the modified Cav-1 peptides for at least 5 days under physiological conditions. In some embodiments, the formulation releases the modified Cav-1 peptides for at least 7 days under physiological conditions. In some embodiments, the formulation releases the modified Cav-1 peptides for at least 14 days under physiological conditions. In some embodiments, the formulation releases the modified Cav-1 peptides for at least 21 days under physiological conditions. In some embodiments, the formulation releases the modified Cav-1 peptides for at least 28 days under physiological conditions. In some embodiments, the formulation releases the modified Cav-1 peptides for at least 35 days under physiological conditions. In some embodiments, the formulation releases the modified Cav-1 peptides for at least 42 days under physiological conditions. In some embodiments, the release of the modified Cav-1 peptides under physiological condition is measured by HPLC after the formulation is placed in a medium that simulates the physiological condition (such as PBS). In some embodiments, the formulation is placed in a medium that simulates the physiological condition while stirring or with oscillation. In some embodiments, the physiological condition is in an aqueous buffer in the range of about pH 6 to about pH 8 at about 37° C. In some embodiments, the physiological condition is in an aqueous buffer of about pH 7.4 at about 37° C.

In some embodiments, a single dose of the formulation described herein releases the modified Cav-1 peptides under physiological conditions at a daily average of about mg of the peptide per 1 kg of the subject's weight (1 mg/kg) to about 3 mg/kg for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, or any value therein. In some embodiments, a single dose of the formulation releases the modified Cav-1 peptides under physiological conditions at a daily average of about 1 mg/kg to about 3 mg/kg for at least 1 week, 2 weeks, 3 weeks, 4 weeks, 5 weeks, 6 weeks, 7 weeks, 8 weeks, 9 weeks, 10 weeks, 11 weeks, 12 weeks, or any value therein. In some embodiments, the physiological condition is in an aqueous buffer in the range of about pH 6 to about pH 8 at about 37° C. In some embodiments, the physiological condition is in an aqueous buffer of about pH 7.4 at about 37° C. In some embodiments, the formulation comprises SAIB.

In some embodiments, a single dose of the formulation as described herein releases the modified Cav-1 peptides for at least 2 days under physiological conditions at a daily average of about 1 mg of the peptide per 1 kg of the subject's weight (1 mg/kg per day) to about 3 mg/kg. In some embodiments, a single dose of the formulation releases the modified Cav-1 peptides for at least 5 days under physiological conditions at a daily average of about 1 mg/kg to about 3 mg/kg. In some embodiments, a single dose of the formulation releases the modified Cav-1 peptides for at least 7 days under physiological conditions at a daily average of about 1 mg/kg to about 3 mg/kg. In some embodiments, a single dose of the formulation releases the modified Cav-1 peptides for at least 14 days under physiological conditions at a daily average of about 1 mg/kg to about 3 mg/kg. In some embodiments, a single dose of the formulation releases the modified Cav-1 peptides for at least 21 days under physiological conditions at a daily average of about 1 mg/kg to about 3 mg/kg. In some embodiments, a single dose of the formulation releases the modified Cav-1 peptides for at least 28 days under physiological conditions at a daily average of about 1 mg/kg to about 3 mg/kg. In some embodiments, a single dose of the formulation releases the modified Cav-1 peptides for at least 35 days under physiological conditions at a daily average of about 1 mg/kg to about 3 mg/kg. In some embodiments, a single dose of the formulation releases the modified Cav-1 peptides for at least 42 days under physiological conditions at a daily average of about 1 mg/kg to about 3 mg/kg.

In some embodiments, the physiological condition is in an aqueous buffer in the range of about pH 6 to about pH 8 at about 37° C. In some embodiments, the physiological condition is in an aqueous buffer of about pH 7.4 at about 37° C.

In some embodiments, a single dose of the formulation as described herein releases the modified Cav-1 peptides for at least 2 days under physiological conditions at a daily average of about 1 mg of the peptide per 1 kg of the subject's weight (1 mg/kg), about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, or about 3 mg/kg. In some embodiments, a single dose of the formulation as described herein releases the modified Cav-1 peptides for at least 7 days, at least 14 days, at least 21 days, at least 28 days, at least 35 days, or at least 42 days under physiological conditions at a daily average of about 1 mg of the peptide per 1 kg of the subject's weight (1 mg/kg), about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, or about to 3 mg/kg. In some embodiments, the physiological condition is in an aqueous buffer in the range of about pH 6 to about pH 8 at about 37° C. In some embodiments, the physiological condition is in an aqueous buffer of about pH 7.4 at about 37° C.

### IV. Methods of Using Formulations for Treatment

In some embodiments, the modified Cav-1 peptide or pharmaceutical formulation thereof is used to treat or prevent a fibrotic disease or disorder. In some embodiments, the modified Cav-1 peptide is used to treat or prevent a fibrotic disease or disorder such as, interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

In some embodiments, the present disclosure provides a method of treating or preventing a fibrotic disease or disorder in a subject, wherein the method comprises administering to the subject an effective amount of a modified Cav-1 peptide or pharmaceutical formulation thereof. In some embodiments, the modified Cav-1 peptides or pharmaceutical formulations thereof are used in the treatment or prevention of a disease or disorder of the lungs in a subject. In some embodiments, the modified Cav-1 peptide is used to treat or prevent a lung disease or disorder such as, for example, acute lung injury (ALI), chronic lung injury, acute respiratory distress syndrome (ARDS), chronic obstructive pulmonary disease (COPD), asthma, interstitial lung disease, pulmonary fibrosis, pneumonia, hypersensitivity pneumonitis, bronchiolitis, sarcoidosis, scleroderma, or pulmonary infection. In some embodiments, the modified Cav-1 peptide is used to treat or prevent a lung disease or disorder in a subject that is elderly or of advanced age. In some embodiments, the elderly subject has interstitial lung disease, e.g., idiopathic pulmonary fibrosis.

In some embodiments, the modified Cav-1 peptides or formulations of the present disclosure are used to treat or prevent a pulmonary infection, e.g., a bacterial, viral, or fungal infection, in a subject. In some embodiments, the pulmonary infection causes one or more lung diseases or disorders in a subject, including but not limited to, ALI, ARDS, COPD, asthma, interstitial lung disease, lung fibrosis, pneumonia, hypersensitivity pneumonitis, bronchiolitis, sarcoidosis, and scleroderma.

In some embodiments, the modified Cav-1 peptides or pharmaceutical formulations of the present disclosure are used to treat or prevent a bacterial infection in a subject. Examples of bacteria that cause pulmonary infections include, but are not limited to, Pseudomonas aeruginosa, Bacillus anthracis, Listeria monocytogenes, Staphylococcus aureus, Streptococcus pneumoniae, Haemophilus influenzae, Enterobacteriaceae, Nocardia, Actinomyces, Moraxella catarrhalis, Klebsiella pneumoniae, Chlamydia trachomatis, Chlamydophilia pneumoniae, Chlamydophilia psittaci, Coxiella burnetti, Salmenellosis, Yersina pestis, Mycobacterium leprae, Mycobacterium africanum, Mycobacterium asiaticum, Mycobacterium aviuin-intracellulaire, Mycobacterium chelonei, Mycobacterium abscessus, Mycobacterium fallax, Mycobacterium fortuitum, Mycobacterium kansasii, Mycobacterium leprae, Mycobacterium malmoense, Mycobacterium shimoidei, Mycobacterium simiae, Mycobacterium szulgai, Mycobacterium xenopi, Mycobacterium tuberculosis, Brucella melitensis, Brucella suis, Brucella abortus, Brucella canis, Legionella pneumonophilia, Francisella tularensis, Pneurnocystis carinii, Mycoplasma pneumoniae, or Burkholderia cepacia. In some embodiments, the bacterial infection causes pneumonia in the subject.

In some embodiments, the modified Cav-1 peptides or pharmaceutical formulations of the present disclosure are used to treat or prevent a viral infection in a subject. In some embodiments, the modified Cav-1 peptide is used to treat or prevent an infection in a subject caused by a double-stranded DNA (dsDNA) virus, a single-stranded DNA (ssDNA) virus, a single-stranded RNA (ssRNA) virus, or a double-stranded RNA (dsRNA) virus. In some embodiments, the ssRNA virus is a positive-sense ssRNA virus (+ssRNA). In some embodiments, the ssRNA virus is a negative-sense ssRNA virus (−ssRNA). Examples of viruses that cause pulmonary infections include, but are not limited to, coronaviruses (e.g., SARS-CoV-1, SARS-CoV-2, or MERS-CoV), influenza, respiratory syncytial virus, metapneumovirus, bocavirus, parainfluenza, rhinovirus, enterovirus, norovirus, adenovirus, varicella-zoster virus, hantavirus, parechovirus, Epstein-Barr virus, herpes simplex virus, mimivirus, cytomegalovirus, torquetenovirus, and Middle East Respiratory Syndrome coronavirus. In some embodiments, the viral infection causes pneumonia in the subject. In some embodiments, the viral infection causes lung fibrosis in the subject. In some embodiments, the viral infection causes bronchiolitis in the subject. In some embodiments, the viral infection causes ALI or ARDS in the subject. In some embodiments, the viral infection causes interstitial lung disease in the subject. In some embodiments, the viral infection causes asthma in the subject. In some embodiments, the viral infection causes sarcoidosis in the subject. In some embodiments, the viral infection causes scleroderma in the subject.

In some embodiments, SARS-CoV-1 causes severe acute respiratory syndrome (SARS) in a subject. In some embodiments, the modified Cav-1 peptide or pharmaceutical formulation is used to treat or prevent SARS in a subject. SARS is initially characterized by systemic symptoms of muscle pain, headache, and fever, followed in 2-14 days by the onset of respiratory symptoms, mainly cough, dyspnea, and pneumonia.

In some embodiments, MERS-CoV causes Middle East respiratory syndrome (MERS) in a subject. In some embodiments, the modified Cav-1 peptide or pharmaceutical formulation is used to treat or prevent MERS in a subject. Clinical features of MERS range from asymptomatic or mild disease to acute respiratory distress syndrome and multiorgan failure resulting in death, especially in individuals with underlying comorbidities. No specific drug treatment exists for MERS and infection prevention and control measures are crucial to prevent spread in health-care facilities. See Zumla et al. Lancet 2015; 386(9997):995-1007.

In some embodiments, SARS-CoV-2 causes coronavirus disease 2019 (COVID-19) in a subject. In some embodiments, the modified Cav-1 peptide or pharmaceutical formulation is used to treat or prevent an infection caused by SARS-CoV-2. In some embodiments, a variant of SARS-CoV-2 causes COVID-19 in a subject. In some embodiments, the modified Cav-1 peptide or pharmaceutical formulation is used to treat or prevent an infection caused by a variant of SARS-CoV-2. In some embodiments, the modified Cav-1 peptide or pharmaceutical formulation is used to treat or prevent an infection caused by a SARS-CoV-2 alpha variant. In some embodiments, the modified Cav-1 peptide or pharmaceutical formulation is used to treat or prevent an infection caused by a SARS-CoV-2 beta variant. In some embodiments, the modified Cav-1 peptide or pharmaceutical formulation is used to treat or prevent an infection caused by a SARS-CoV-2 gamma variant. In some embodiments, the modified Cav-1 peptide or pharmaceutical formulation is used to treat or prevent an infection caused by a SARS-CoV-2 delta variant. In some embodiments, the modified Cav-1 peptide or pharmaceutical formulation is used to treat or prevent an infection caused by a SARS-CoV-2 epsilon variant.

In some embodiments, the modified Cav-1 peptide or pharmaceutical formulation is used to treat or prevent an infection caused by a SARS-CoV-2 zeta variant. In some embodiments, the modified Cav-1 peptide or pharmaceutical formulation is used to treat or prevent an infection caused by a SARS-CoV-2 eta variant. In some embodiments, the modified Cav-1 peptide or pharmaceutical formulation is used to treat or prevent an infection caused by a SARS-CoV-2 theta variant. In some embodiments, the modified Cav-1 peptide or pharmaceutical formulation is used to treat or prevent an infection caused by a SARS-CoV-2 iota variant. In some embodiments, the modified Cav-1 peptide or pharmaceutical formulation is used to treat or prevent an infection caused by a SARS-CoV-2 kappa variant. In some embodiments, the modified Cav-1 peptide is used to treat or prevent post-acute COVID-19 caused by a SARS-CoV-2 lambda variant. In some embodiments, the modified Cav-1 peptide is used to treat or prevent post-acute COVID-19 caused by a SARS-CoV-2 mu variant. In some embodiments, the modified Cav-1 peptide is used to treat or prevent post-acute COVID-19 caused by a SARS-CoV-2 omicron variant. n some embodiments, the SARS-CoV-2 variant is B.1.1.7 (also referred to as 501Y.V1 or VOC-202012/01), B.1.1.317, B.1.1.318, B.1.1.529, B.1.351 (also referred to as 501Y.V2), B.1.429, B1.427, B1.1.207, A.23.1, COH.20G/501Y, B.1.525, B.1.526, B.1.617, B.1.618, B. 1.621, C.37, P.1, P.2, or P.3, or a subvariant thereof, or a recombinant form thereof. See Konings et al., Variants of Interest and Concern naming scheme conducive for global discourse. Nature Microbiology (2021). In some embodiments, the subvariant of SARS-CoV-2 variant B.1.1.529 is BA.1 (B1.1.529.1), BA1.1 (B1.1.529.1.1), BA.2 (B1.1.529.2), BA.3 (B1.1.529.3), BA.4 (B1.1.529.4), or BA.5 (B1.1.529.5). In some embodiments, the subvariant of SARS-CoV-2 variant B.1.1.7 is Q.1, Q.2, Q.3, Q.4, Q.5, Q.6, Q.7, or Q.8. In some embodiments, the subvariant of SARS-CoV-2 variant B.1.351 is B.1.351.1, B.1.351.2, B.1.351.3, B.1.351.4, or B.1.351.5. In some embodiments, the subvariant of SARS-CoV-2 variant P.1 is P.1.1, P.1.2, P.1.3, P.1.4, P.1.5, P.1.6, P.1.7, P.1.7.1, P.1.8, P.1.9, P.1.10, P.1.10.1, P.1.10.2, P.1.11, P.1.12, P.1.12.1, P.1.13, P.1.14, P.1.15, P.1.16, P.1.17, or P.1.17.1. In some embodiments, the subvariant of SARS-CoV-2 variant B.1.617 is B.1.617.1, B.1.617.2, or B.1.617.3. In some embodiments, the subvariant of SARS-CoV-2 variant B.1.526 is B.1.526.1. In some embodiments, the subvariant of SARS-CoV-2 variant B.1.621 is B.1.621.1, B.1.621.2, BB.1, or BB.2. In some embodiments, the subvariant of SARS-CoV-2 variant C.37 is C.37.1.

In some embodiments, the modified Cav-1 peptides or pharmaceutical formulations of the present disclosure are used to treat or prevent a fungal infection in a subject. Examples of fungi that cause pulmonary infections include, but are not limited to, Candida (e.g., Candida albicans, Candida glabrata, Candida krusei), Aspergillus, Pneumocystis, Coccidioides (e.g., Coccidioides immitis, Coccidioides posadasii), Blastomyces (e.g., Blastomyces dermatitidis), Histoplasma (e.g., Histoplasma capsulatum), Cryptococcus (e.g., Cryptococcus neoformans, Cryptococcus gattii), Sporothrix (e.g., Sporothrix schenckii), Mucor, and Paracoccidioides. In some embodiments, the fungal infection causes pneumonia in the subject. In some embodiments, the fungal infection causes invasive pulmonary aspergillosis in the subject. In some embodiments, the fungal infection causes allergic asthma, allergic bronchopulmonary aspergillosis, or hypersensitivity pneumonitis in the subject. In some embodiments, the fungal infection causes ARDS. In some embodiments, the fungal infection causes pulmonary fibrosis in the subject. In some embodiments, the fungal infection causes pulmonary oedema in the subject.

In some embodiments, the modified Cav-1 peptides or pharmaceutical formulations of the present disclosure are used to treat or prevent interstitial lung disease in a subject. Interstitial lung disease is a group of disorders that causes fibrosis and inflammation of the interstitium.

In some embodiments, the interstitial lung disease is idiopathic pulmonary fibrosis, lymphangioleiomyomatosis, nonspecific interstitial pneumonia, idiopathic interstitial pneumonia, cryptogenic organizing pneumonia, acute interstitial pneumonia, respiratory bronchiolitis-associated interstitial lung disease, desquamative interstitial pneumonia, lymphocytic interstitial pneumonia, pulmonary sarcoidosis, diffuse alveolar damage, systemic sclerosis, polymyositis, systemic lupus erythematosus, rheumatoid arthritis, drug-induced interstitial lung disease, or occupational interstitial lung disease. In some embodiments, the interstitial lung disease is idiopathic pulmonary fibrosis.

In some embodiments, the modified Cav-1 peptides or pharmaceutical formulations of the present disclosure are used to treat or prevent acute lung injury (ALI) in a subject. ALI is a disorder of acute inflammation that causes disruption of the lung endothelial and epithelial barriers. ALI may be the result of inhalation injury or a consequence of systemic disease, such as sepsis or severe hypovolemic shock. In some embodiments, the ALI is chemical-induced ALI. In some embodiments, the ALI is inhalational smoke induced acute lung injury (ISALI). In some embodiments, the ALI is ARDS.

In some embodiments, the modified Cav-1 peptides or pharmaceutical formulations of the present disclosure are used to treat or prevent ARDS in a subject. ARDS is the most severe form of ALI and is distinguished by the severity of the oxygenation deficit. ARDS is a life-threatening type of lung injury that occurs when fluid builds up in the tiny, elastic air sacs (alveoli) in the lungs. The fluid in the alveoli prevents the lungs from filling up with oxygen resulting in less oxygen reaching the bloodstream and a difficulty in breathing.

In some embodiments, the modified Cav-1 peptide or pharmaceutical formulation is used to treat or prevent cystic fibrosis (CF) in a subject. CF is an inherited disease of the exocrine glands and exocrine sweat glands which primarily affects the digestive and respiratory systems. This disease is usually characterized by chronic respiratory infections, pancreatic insufficiency, abnormally viscid mucus secretions and premature death. CF is characterized by progressive airflow obstruction. Subsets of individuals with CF also develop airway hyper-responsiveness to inhaled cholinergic agonists (Weinberger, 2002 and Mitchell et al., 1978) and reversibility of airflow limitation in response to bronchodilators (van Haren et al., 1991 and van Haren et al., 1992). The presence of bronchial hyper-responsiveness and airway obstruction suggest a possible shared etiology of disease between CF and other diseases of airway narrowing such as asthma or COPD where airway smooth muscle dysfunction is thought to contribute to the disease processes.

In some embodiments, the modified Cav-1 peptide or pharmaceutical formulation is used to treat or prevent COPD in a subject. COPD is a term used to classify two major airflow obstruction disorders: chronic bronchitis and emphysema. Chronic bronchitis is inflammation of the bronchial airways. The bronchial airways connect the trachea with the lungs. When inflamed, the bronchial tubes secrete mucus, causing a chronic cough. In emphysema, the alveolar sacs are overinflated as a result of damage to the elastin skeleton of the lung.

Inflammatory cells in emphysematous lung release elastase enzymes, which degrade or damage elastin fibers within the lung matrix. Emphysema has a number of causes, including smoking, exposure to environmental pollutants, alpha-one antitrypsin deficiency, and aging.

In some embodiments, the modified Cav-1 peptides or pharmaceutical formulations disclosed herein are used to treat or prevent bronchiolitis in a subject. Bronchiolitis is most commonly caused by viral lower respiratory tract infections, and primarily characterized by acute inflammation, edema, necrosis of epithelial cells lining small airways, and increased mucus production (Ralston et al., 2014). Signs and symptoms typically begin with rhinitis and cough, which may progress to tachypnea, wheezing, rales, use of accessory muscles, and/or nasal flaring.

In some embodiments, the modified Cav-1 peptides or pharmaceutical formulations disclosed herein are used to treat or prevent bronchiolitis obliterans in a subject. Bronchiolitis obliterans is a progressive airflow reduction as a result of abnormal remodeling of the small airways in the lungs (Meyer et al., 2014). Bronchiolitis obliterans is a major complication of lung transplantations, and is often used to describe a delayed allograft dysfunction that results in persistent decline in forced expiratory volume and force that is not caused by other known causes (Meyer et al., 2014).

In some embodiments, the modified Cav-1 peptides or pharmaceutical formulations disclosed herein are used to treat or prevent asthma in a subject. The term “asthma” may refer to acute asthma, chronic asthma, intermittent asthma, mild persistent asthma, moderate persistent asthma, severe persistent asthma, chronic persistent asthma, mild to moderate asthma, mild to moderate persistent asthma, mild to moderate chronic persistent asthma, allergic (extrinsic) asthma, non-allergic (intrinsic) asthma, nocturnal asthma, bronchial asthma, exercise induced asthma, occupational asthma, seasonal asthma, silent asthma, gastroesophageal asthma, idiopathic asthma and cough variant asthma. During asthma, the airways are persistently inflamed and may occasionally spasm.

In some embodiments, the modified Cav-1 peptides or pharmaceutical formulations disclosed herein are used to treat or prevent hypersensitivity pneumonitis in a subject. Hypersensitivity pneumonitis is a complex syndrome caused by the inhalation of a variety of antigens in susceptible and sensitized individuals. These antigens are found in the environment, mostly derived from bird proteins and fungi. Hypersensitivity pneumonitis is characterized by an exaggerated humoral and cellular immune response affecting the small airways and lung parenchyma. Hypersensitivity pneumonitis can be classified into acute, chronic non-fibrotic and chronic fibrotic forms. Acute hypersensitivity pneumonitis results from intermittent, high-level exposure to the inducing antigen, usually within a few hours of exposure, whereas chronic hypersensitivity pneumonitis mostly originates from long-term, low-level exposure (usually to birds or molds in the home), is not easy to define in terms of time, and may occur within weeks, months or even years of exposure. Some patients with fibrotic hypersensitivity pneumonitis may evolve to a progressive phenotype, even with complete exposure avoidance. See Costabel et al., Nature Reviews Disease Primers 2020; 6(65).

In some embodiments, the modified Cav-1 peptide or pharmaceutical formulation is used to treat or prevent systemic sclerosis or scleroderma in a subject. Systemic sclerosis is a systemic autoimmune disease that is characterized by endothelial dysfunction resulting in a small-vessel vasculopathy, fibroblast dysfunction with resultant excessive collagen production and fibrosis, and immunological abnormalities. The classification of systemic sclerosis is subdivided based on the extent of skin involvement into diffuse cutaneous sclerosis, limited cutaneous sclerosis or systemic sclerosis sine scleroderma. While virtually any organ system may be involved in the disease process, fibrotic and vascular pulmonary manifestations of systemic sclerosis, including interstitial lung disease and pulmonary hypertension, are the leading cause of death. While certain pulmonary manifestations may occur more commonly in a subset of systemic sclerosis (i.e., ILD is more common in diffuse cutaneous sclerosis, while pulmonary hypertension is more common in limited cutaneous sclerosis), all of the known pulmonary manifestations reported have been described in each of the subsets of disease. Pulmonary disease can even occur in systemic sclerosis with no skin involvement (an entity known as scleroderma sine scleroderma). See Solomon et al., Eur Respir Rev 2013; 22(127):6-19.

In some embodiments, the modified Cav-1 peptide or pharmaceutical formulation is used to treat or prevent sarcoidosis in a subject. Sarcoidosis is a multisystem disorder that is characterized by noncaseous epithelioid cell granulomas, which may affect almost any organ. Thoracic involvement is common and accounts for most of the morbidity and mortality associated with the disease. Thoracic abnormalities are observed in approximately 90% of patients with sarcoidosis, and an estimated 20% develop chronic lung disease leading to pulmonary fibrosis. Pulmonary sarcoidosis may manifest with various patterns: Bilateral hilar lymph node enlargement is the most common finding, followed by interstitial lung disease. The most typical findings of pulmonary involvement are micronodules with a perilymphatic distribution, fibrotic changes, and bilateral perihilar opacities. Atypical manifestations, such as mass-like or alveolar opacities, honeycomb-like cysts, miliary opacities, mosaic attenuation, tracheobronchial involvement, and pleural disease, and complications such as aspergillomas, also may be seen. See Criado et al., Chest Imaging 2010; 30(6).

In some embodiments, the present disclosure provides a method of treating or preventing a lung disease or disorder in an elderly subject, wherein the method comprises administering to the elderly subject an effective amount of a modified Cav-1 peptide or pharmaceutical formulation thereof.

In some embodiments, the modified Cav-1 peptides or pharmaceutical formulations thereof are used in the treatment or prevention of a disease or disorder of the kidney (e.g., chronic kidney disease) in a subject. In some embodiments, the modified Cav-1 peptides or pharmaceutical formulations thereof are used in the treatment or prevention of a disease or disorder of the lungs (e.g., idiopathic pulmonary fibrosis) in a subject. In some embodiments, the subject is elderly or of advanced age.

In some embodiments, the modified Cav-1 peptide formulations is used to treat or prevent a kidney disease or disorder such as, for example, chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, Wegener's granulomatosis, or acute kidney injury. In some embodiments, the kidney disease is characterized by fibrosis. In some embodiments, the kidney disease or disorder is acute. In some embodiments, the kidney disease or disorder is chronic. In some embodiments, the subject is elderly or of advanced age. In some embodiments, the kidney disease or disorder in the subject is caused by an infection, such as, for example, a viral, bacterial, fungal, or parasitic infection. In some embodiments, the kidney disease or disorder in the subject is caused by high blood pressure (hypertension). In some embodiments, the kidney disease or disorder in the subject is caused by diabetes. In some embodiments, the kidney disease or disorder in the subject is caused by overuse of drugs, such as over-the counter pain killers and heroine. In some embodiments, the subject has hypertension or diabetes.

In some embodiments, the modified Cav-1 peptides or pharmaceutical formulations thereof are used in delaying the progression of a disease or disorder of the kidney in a subject. In some embodiments, the modified Cav-1 peptides or pharmaceutical formulations thereof are used in improving progression free survival. In some embodiments, the modified Cav-1 peptides or pharmaceutical formulations thereof are used to prolong survival of the subject.

In some embodiments, the modified Cav-1 peptides or pharmaceutical formulations of the present disclosure are used to treat or prevent chronic kidney disease in a subject. Chronic kidney disease is a gradual and progressive loss of the ability of the kidneys to excrete wastes, concentrate urine, and conserve electrolytes. The progressive loss of renal function occurs as a consequence of the deposition of fibrous tissue between the functional units of the kidney or nephrons (interstitial fibrosis) as well as the ongoing replacement of the filtration surface by fibrous tissue (glomerular sclerosis). Kidney fibrosis is a pathological hallmark of chronic kidney disease and a major contributing factor of progression to end-stage renal disease. In one embodiment, the chronic kidney disease is chronic kidney fibrosis.

In some embodiments, the modified Cav-1 peptides or pharmaceutical formulations of the present disclosure are used to treat or prevent end-stage renal disease in a subject. End-stage renal disease is the final stage of chronic kidney disease where the kidneys have ceased functioning and the individual requires long-term dialysis or a kidney transplant to survive.

In some embodiments, the modified Cav-1 peptides or pharmaceutical formulations of the present disclosure are used to treat or prevent focal segmental glomerulosclerosis in a subject. Focal segmental glomerulosclerosis is a kidney disease characterized by scarring of the glomerulus causing loss of protein into the urine.

In some embodiments, the modified Cav-1 peptides or pharmaceutical formulations of the present disclosure are used to treat or prevent glomerulonephritis in a subject. Glomerulonephritis, also referred to as glomerular disease, is a type of kidney disease in which the glomeruli are damaged and cannot remove waste and fluid properly from the body. In some embodiments, the glomerulonephritis is acute glomerulonephritis. In some embodiments, the glomerulonephritis is chronic glomerulonephritis.

In some embodiments, the modified Cav-1 peptides or pharmaceutical formulations of the present disclosure are used to treat or prevent polycystic kidney disease in a subject. Polycystic kidney disease is an inherited disorder in which clusters of cysts develop primarily within the kidneys causing the kidneys to enlarge and lose function overtime.

In some embodiments, the modified Cav-1 peptides or pharmaceutical formulations of the present disclosure are used to treat or prevent IgA nephropathy in a subject. IgA nephropathy, also referred to as Berger's disease, is a kidney disease that occurs when the immunoglobulin IgA accumulates in the kidneys resulting in local inflammation that can prevent the ability of the kidneys to filter waste from the blood.

In some embodiments, the modified Cav-1 peptides or pharmaceutical formulations of the present disclosure are used to treat or prevent lupus nephritis in a subject. Lupus nephritis is a type of glomerulonephritis that constitutes one of the most severe organ manifestations of the systemic lupus erythematosus and occurs when the immune system attacks the kidneys.

In some embodiments, the modified Cav-1 peptides or pharmaceutical formulations of the present disclosure are used to treat or prevent nephrotic syndrome in a subject. Nephrotic syndrome is a kidney disorder that causes the body to excrete too much protein into the urine. Nephrotic syndrome is often caused by damage to small blood vessels in the kidneys that filter waste and excess water from the blood.

In some embodiments, the modified Cav-1 peptides or pharmaceutical formulations of the present disclosure are used to treat or prevent Alport syndrome in a subject. Alport syndrome is a genetic condition characterized by progressive kidney disease and abnormalities of the inner ear and the eye. There are three genetic types: X-linked Alport syndrome (XLAS), autosomal recessive Alport syndrome (ARAS), and autosomal dominant Alport syndrome (ADAS). XLAS is caused by variants in the COL4A5 gene, ARAS is caused by variants in both copies of either the COL4A3 or the COL4A4 gene, and ADAS is caused by variants in one copy of the COL4A3 or COL4A4 gene. Individuals with Alport syndrome present with chronic glomerular dysfunction, renal inflammation, and fibrosis, which are the hallmarks of chronic kidney disease, and progress to end-stage kidney disease. Those afflicted with Alport syndrome can also develop progressive hearing loss of varying severity and abnormalities of the eyes that usually do not result in impaired vision.

In some embodiments, the modified Cav-1 peptides or pharmaceutical formulations of the present disclosure are used to treat or prevent amyloidosis in a subject. Amyloidosis occurs when amyloid builds up in tissues and organs and interferes with normal function. Amyloid deposits damage the kidney affecting the ability of the kidney to filter wastes and break down proteins. In some embodiments, the amyloidosis is primary amyloidosis. In some embodiments, the amyloidosis is dialysis-related amyloidosis.

In some embodiments, the modified Cav-1 peptides or pharmaceutical formulations of the present disclosure are used to treat or prevent Goodpasture syndrome in a subject. Goodpasture syndrome, also referred to as anti-glomerular basement membrane disease, is an autoimmune disease in which antibodies attack the basement membrane of the lungs and kidneys leading to pulmonary hemorrhage, glomerulonephritis, and kidney failure.

In some embodiments, the modified Cav-1 peptides or pharmaceutical formulations of the present disclosure are used to treat or prevent granulomatosis with polyangiitis in a subject. Granulomatosis with polyangiitis, also referred to as Wegener's granulomatosis, is an autoimmune disease involving granulomatous inflammation, necrosis, and vasculitis that most frequently targets the lungs and kidneys.

In some embodiments, the modified Cav-1 peptides or pharmaceutical formulations of the present disclosure are used to treat or prevent acute kidney injury in a subject. Acute kidney injury, also referred to as acute renal failure, is a sudden loss of excretory kidney function. Acute kidney injury is defined by serum creatine and urine output levels with a duration of less than one week.

In some embodiments, the modified Cav-1 peptide or pharmaceutical formulation is used to treat or prevent a kidney infection. In some embodiments, the modified Cav-1 peptide is used to treat or prevent pyelonephritis. Pyelonephritis is a type of urinary tract infection where one or both kidneys become infected. In some embodiments, the pyelonephritis is caused by a bacteria or virus, such as, for example, Escherichia coli, Klebsiella, Proteus, Pseudomonas, Enterococcus, or Staphylococcus saprophyticus.

In some embodiments, the modified Cav-1 peptide or pharmaceutical formulation is used to treat or prevent a kidney disease or disorder resulting from microbial infection in a subject. In some embodiments, the microbial infection is a bacterial, viral, fungal, or parasitic infection. In some embodiments, the kidney disease or disorder is caused by Streptococcus pyogenes, Staphylococcus (aureus, epidermidis), Salmonella (typhi, paratyphi), Escherichia coli, Leptospira, Mycobacterium tuberculosis, Mycobacterium leprae, Ligionella spp., Yersinia enterocolitica, Brucella species, Campylobacter jejuni, Corynebacterium diphtheriae, Klebsiella, Proteus, Pseudomonas, Enterococcus, Staphylococcus saprophyticus, SARS-CoV-1, SARS-CoV-2, dengue virus, hantavirus, Varicella-zoster virus, parvovirus, hepatitis A virus, hepatitis B virus, hepatitis E virus, cytomegalovirus, Epstein-Barr virus, human immunodeficiency virus, and/or hepatitis C virus.

In some embodiments, the modified Cav-1 peptide or pharmaceutical formulation is used to treat or prevent a kidney disease or disorder resulting from infection with SARS-CoV-2. In some embodiments, SARS-CoV-2 causes acute kidney injury. In some embodiments, SARS-CoV-2 causes chronic kidney injury. In some embodiments, SARS-CoV-2 causes chronic kidney disease. In some embodiments, the SARS-CoV-2 causes kidney fibrosis. In some embodiments, the SARS-CoV-2 causes kidney failure.

In some embodiments, the kidney disease or disorder is chronic kidney disease. In some embodiments, the kidney disease or disorder is Alport syndrome. In some embodiments, the modified Cav-1 peptides or pharmaceutical formulations thereof are used to improve kidney function in a subject with a kidney disease or disorder. In some embodiments, the modified Cav-1 peptide or pharmaceutical formulation thereof improves kidney function by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof. In some embodiments, an improvement in kidney function signifies a decrease in fibrotic glomeruli, a decrease in blood urea nitrogen, a decrease in blood creatinine, an increase in blood albumin, a decrease in urine albumin to creatinine ratio, and/or an increase in glomerular filtration rate.

In some embodiments, the modified Cav-1 peptides or pharmaceutical formulations thereof decrease the number of fibrotic glomeruli in a subject with a kidney disease or disorder. In some embodiments, the modified Cav-1 peptide or pharmaceutical formulation thereof decreases the number of fibrotic glomeruli by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof.

In some embodiments, the subject with a kidney disease or disorder has reduced caveolin-1 expression in the kidney compared to a subject without a kidney disease or disorder (e.g., a normal, healthy subject). In some embodiments, caveolin-1 expression is reduced in the glomeruli of the subject with a kidney disease or disorder compared to the subject without a kidney disease or disorder. In some embodiments, caveolin-1 expression is reduced in endothelial cells of the kidney in the subject with a kidney disease or disorder compared to the subject without a kidney disease or disorder. In some embodiments, caveolin-1 expression is reduced in epithelial cells of the kidney in the subject with a kidney disease or disorder compared to the subject without a kidney disease or disorder. In some embodiments, caveolin-1 expression is reduced in podocytes of the kidney in the subject with a kidney disease or disorder compared to the subject without a kidney disease or disorder. In some embodiments, caveolin-1 expression in the kidney is reduced in the subject with a kidney disease or disorder by at least about 10%, at least about 20%, at least about 30%, at least about 40%, at least about 50%, at least about 60%, at least about 70%, at least about 80%, or at least about 90% compared to the subject without a kidney disease or disorder. In some embodiments, the epithelial cells are parietal epithelial cells lining Bowman's capsule.

In some embodiments, the modified Cav-1 peptides or pharmaceutical formulations thereof decrease endothelial cell death in a subject with a kidney disease or disorder. In some embodiments, the modified Cav-1 peptides or pharmaceutical formulations thereof decrease epithelial cell death in a subject with a kidney disease or disorder. In some embodiments, the modified Cav-1 peptides or pharmaceutical formulations thereof decrease podocyte cell death in a subject with a kidney disease or disorder. In some embodiments, the modified Cav-1 peptide or pharmaceutical formulations thereof decrease cell death by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof.

In some embodiments, the modified Cav-1 peptides or pharmaceutical formulations thereof increases endothelial cell survival in a subject with a kidney disease or disorder. In some embodiments, the modified Cav-1 peptides or pharmaceutical formulations thereof increase epithelial cell survival in a subject with a kidney disease or disorder. In some embodiments, the modified Cav-1 peptides or pharmaceutical formulations thereof increase podocyte survival in a subject with a kidney disease or disorder. In some embodiments, the modified Cav-1 peptides or pharmaceutical formulations thereof increase cell survival by at least about 10%, at least about 20%, at least about 30%, at least about 40%, at least about 50%, at least about 60%, at least about 70%, at least about 80%, or at least about 90% compared to the subject before treatment with the modified Cav-1 peptides or pharmaceutical formulations thereof.

In some embodiments, the modified Cav-1 peptides or pharmaceutical formulations thereof promotes kidney regeneration in a subject with a kidney disease or disorder. In some embodiments, the modified Cav-1 peptides or pharmaceutical formulations thereof promote regeneration of renal vessels, glomeruli, and/or tubules in the kidney of the subject. In some embodiments, the modified Cav-1 peptides or pharmaceutical formulations thereof promotes regeneration of epithelial cells, endothelial cells, tubular cells, and/or podocytes in the kidney of the subject.

In some embodiments, the modified Cav-1 peptides or pharmaceutical formulations thereof increases endothelial cell proliferation in a subject with a kidney disease or disorder. In some embodiments, the modified Cav-1 peptides or pharmaceutical formulations thereof increases epithelial cell proliferation in a subject with a kidney disease or disorder. In some embodiments, the modified Cav-1 peptides or pharmaceutical formulations thereof increases podocyte proliferation in a subject with a kidney disease or disorder. In some embodiments, the modified Cav-1 peptide or pharmaceutical formulations thereof increases cell proliferation by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof.

In some embodiments, the modified Cav-1 peptides or pharmaceutical formulations thereof decrease blood urea nitrogen in a subject with a kidney disease or disorder. In some embodiments, a high blood urea nitrogen value indicates kidney injury or disease in a subject. In general, a blood urea nitrogen level ranging from 6 mg/dl to 24 mg/dl is considered normal in humans. In some embodiments, the modified Cav-1 peptide or pharmaceutical formulation thereof decreases blood urea nitrogen by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof. In some embodiments, the modified Cav-1 peptide or pharmaceutical formulation thereof decreases blood urea nitrogen in a subject to less than about 50 mg/dl, less than about 45 mg/dl, less than about 40 mg/dl, less than about 35 mg/dl, less than about 30 mg/dl, less than about 25 mg/dl, or less than about 20 mg/dl. In some embodiments, the modified Cav-1 peptide or pharmaceutical formulation thereof decreases blood urea nitrogen to less than about 20 mg/dl.

In some embodiments, the modified Cav-1 peptides or pharmaceutical formulations thereof decrease blood creatinine in a subject with a kidney disease or disorder. In some embodiments, a high blood creatinine value indicates kidney injury or disease in a subject. In general, a blood creatinine value of greater than 1.2 mg/dl in women and 1.4 mg/dl in men signifies that the kidneys are not functioning properly. In some embodiments, the modified Cav-1 peptide or pharmaceutical formulation thereof decreases blood creatinine by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof. In some embodiments, the modified Cav-1 peptide or pharmaceutical formulation thereof decreases blood creatinine in a subject to less than about 4 mg/dl, less than about 3.5 mg/dl, less than about 3.25 mg/dl, less than about 3 mg/dl, less than about 2.75 mg/dl, less than about 2.5 mg/dl, less than about 2.25 mg/dl, less than about 2 mg/dl, less than about 1.75 mg/dl, less than about 1.5 mg/dl, or less than about 1.25 mg/dl. In some embodiments, the modified Cav-1 peptide or pharmaceutical formulation thereof decreases blood urea nitrogen to less than about 1.5 mg/dl.

In some embodiments, the modified Cav-1 peptides or pharmaceutical formulations thereof increase blood albumin in a subject with a kidney disease or disorder. In some embodiments, a low blood albumin value may indicate kidney injury or disease in a subject. The normal level of albumin in the blood is 3.5 g/dL to 5 g/dL. In some embodiments, the modified Cav-1 peptide or pharmaceutical formulation thereof increases blood albumin by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof. In some embodiments, the modified Cav-1 peptide or pharmaceutical formulation thereof increases blood albumin in a subject to greater than about 1.5 g/dl, greater than about 1.75 g/dl, greater than about 2 g/dl, greater than about 2.25 g/dl, greater than about 2.5 g/dl, greater than about 2.75 g/dl, greater than about 3 g/dl, or greater than about 3.5 g/dl. In some embodiments, the modified Cav-1 peptide or pharmaceutical formulation thereof increases blood albumin to greater than about 3.5 g/dl.

In some embodiments, the modified Cav-1 peptides or pharmaceutical formulations thereof decrease the urine albumin to creatinine ratio in a subject with a kidney disease or disorder. The urine albumin to creatinine ratio helps to identify kidney injury or disease in a subject. A ratio of albumin to creatinine of less than 30 mg/g is considered normal; a ratio of 30-300 mg/g signifies microalbuminuria, and values above 300 mg/g signify macroalbuminuria in humans. In some embodiments, the modified Cav-1 peptide or pharmaceutical formulation thereof decreases the urine albumin to creatinine ratio by at least about 10%, at least about 20%, at least about 30%, at least about 40%, at least about 50%, at least about 60%, at least about 70%, at least about 80%, or at least about 90% compared to the subject before treatment with the modified Cav-1 peptide or pharmaceutical formulation thereof. In some embodiments, the modified Cav-1 peptide or pharmaceutical formulation thereof decreases the urine albumin to creatinine ratio in a subject to less than about 300 mg/g, less than about 250 mg/g, less than about 200 mg/g, less than about 150 mg/g, less than about 100 mg/g, less than about 75 mg/g, less than about 50 mg/g, less than about 40 mg/g, less than about 30 mg/g, or less than about 25 mg/g. In some embodiments, the modified Cav-1 peptide or pharmaceutical formulation thereof decreases the urine albumin to creatinine ratio to less than about 30 mg/g.

In some embodiments, the modified Cav-1 peptides or pharmaceutical formulations thereof increase the glomerular filtration rate in a subject with a kidney disease or disorder.

The glomerular filtration rate measures how well the kidneys are filtering the blood to remove waste and extra water to make urine. A glomerular filtration rate above 90 mL/min/1.73 m² is considered normal, while a glomerular filtration rate of less than 60 mL/min/1.73 m² may signify kidney injury or disease. A glomerular filtration rate below 15 mL/min/1.73 m² may signify kidney failure. In some embodiments, the modified Cav-1 peptide or pharmaceutical formulation thereof increases the glomerular filtration rate by at least about 10%, at least about 20%, at least about 30%, at least about 40%, at least about 50%, at least about 60%, at least about 70%, at least about 80%, at least about 90%, or at least about 100% compared to the subject before treatment with the modified Cav-1 peptide or pharmaceutical formulation thereof. In some embodiments, the modified Cav-1 peptide or pharmaceutical formulation thereof increases the glomerular filtration rate in a subject to greater than about 30 mL/min/1.73 m², greater than about 40 mL/min/1.73 m², greater than about 50 mL/min/1.73 m², greater than about 60 mL/min/1.73 m², greater than about 70 mL/min/1.73 m², greater than about 80 mL/min/1.73 m², or greater than about 90 mL/min/1.73 m². In some embodiments, the modified Cav-1 peptide or pharmaceutical formulation thereof increases the glomerular filtration rate in a subject to greater than about 60 mL/min/1.73 m².

In some embodiments, the modified Cav-1 peptides or pharmaceutical formulations thereof are used to preserve kidney function in a subject with a kidney disease or disorder. The term “preserve” as used herein refers to maintaining kidney function or preventing a further decline in kidney function in a subject with a kidney disease or disorder. In some embodiments, the modified Cav-1 peptide or pharmaceutical formulation thereof preserves kidney function as measured by blood urine nitrogen, blood creatinine, blood albumin, urine albumin to creatinine ratio, and/or glomerular filtration rate, wherein these measurements remain stable upon treatment with the modified Cav-1 peptide or pharmaceutical formulation thereof.

In some embodiments, the modified Cav-1 peptides or pharmaceutical formulations thereof are used in the treatment or prevention of a disease or disorder in an elderly subject. As used herein, the terms “elderly” or “advanced age” refers to a subject 55 years of age or older. In some embodiments, the elderly subject is about 55 years old, about 60 years old, about 65 years old, about 70 years old, about 75 years old, about 80 years old, about 90 years old, about 95 years old, or about 100 years old. In some embodiments, the elderly subject has an increased susceptibility to a disease or disorder described herein compared to a subject of a younger age. In some embodiments, the elderly subject has a fibrotic disease or disorder, e.g., idiopathic pulmonary fibrosis.

In some embodiments, the elderly subject has reduced caveolin-1 expression compared to a younger subject (e.g., a young adult or middle-age subject). In some embodiments, caveolin-1 expression is reduced in the elderly subject by at least about 10%, at least about 20%, at least about 30%, at least about 40%, at least about 50%, at least about 60%, at least about 70%, at least about 80%, or at least about 90% compared to the younger subject.

In some embodiments, the present disclosure provides a method of treating or preventing a fibrotic disease or disorder in an elderly subject, wherein the method comprises administering to the elderly subject an effective amount of a modified Cav-1 peptide or pharmaceutical formulation thereof.

In some embodiments of any one of the methods disclosed herein, the formulation comprising the modified Cav-1 peptide as disclosed herein comprises a modified Cav-1 peptide comprising an amino acid sequence of any one of SEQ ID NOs: 2-111. In some embodiments of any one of the methods disclosed herein, the formulation comprising the modified Cav-1 peptide as disclosed herein comprises a modified Cav-1 peptide comprising an amino acid sequence of any one of SEQ ID NOs: 2-10. In some embodiments of any one of the methods disclosed herein, the formulation comprising the modified Cav-1 peptide as disclosed herein comprises a modified Cav-1 peptide comprising an amino acid sequence of SEQ ID NO: 3. In some embodiments of any one of the methods disclosed herein, the formulation comprising the modified Cav-1 peptide as disclosed herein comprises a modified Cav-1 peptide comprising an amino acid sequence of SEQ ID NO: 8. In some embodiments of any one of the methods disclosed herein, the formulation comprising the modified Cav-1 peptide as disclosed herein comprises a modified Cav-1 peptide comprising at least one amino acid substitution, deletion of insertion relative to the amino acid sequence of FTTFTVT (SEQ ID NO: 3), wherein the modified Cav-1 peptide maintains the biological activity of Cav-1.

In some embodiments of any one of the methods disclosed herein, the formulation comprising the modified Cav-1 peptide as disclosed herein comprises an effective amount of a modified Cav-1 peptide comprising an amino acid sequence of any one of SEQ ID NOs: 2-111. In some embodiments of any one of the methods disclosed herein, the formulation comprising the modified Cav-1 peptide as disclosed herein comprises an effective amount of a modified Cav-1 peptide comprising an amino acid sequence of any one of SEQ ID NOs: 2-10. In some embodiments of any one of the methods disclosed herein, the formulation comprising the modified Cav-1 peptide as disclosed herein comprises an effective amount of a modified Cav-1 peptide comprising an amino acid sequence of SEQ ID NO: 3. In some embodiments of any one of the methods disclosed herein, the formulation comprising the modified Cav-1 peptide as disclosed herein comprises an effective amount of a modified Cav-1 peptide comprising an amino acid sequence of SEQ ID NO: 8. In some embodiments of any one of the methods disclosed herein, the formulation comprising the modified Cav-1 peptide as disclosed herein comprises an effective amount of a modified Cav-1 peptide comprising at least one amino acid substitution, deletion of insertion relative to the amino acid sequence of FTTFTVT (SEQ ID NO: 3), wherein the modified Cav-1 peptide maintains the biological activity of Cav-1.

The present disclosure contemplates all modes of administration, dosing, or frequency of dosing adequate to treat or prevent the disease or disorder in a subject. Effective doses may also be extrapolated from dose-response curves derived from in vitro or animal model test bioassays or systems.

In some embodiments, the modified Cav-1 peptides or pharmaceutical formulations thereof is administered systemically. In some embodiments, the modified Cav-1 peptide or pharmaceutical formulation thereof is administered intravenously, intrathecally, subcutaneously, and/or intraperitoneally.

In some embodiments, the modified Cav-1 peptide or pharmaceutical formulation thereof is delivered locally to the airway of a subject, such as administration of a nebulized formulation using a nebulizer or a dry powder formulation using a dry powder inhaler. In some embodiments, the modified Cav-1 peptide or pharmaceutical formulation thereof is administered to the lungs of a subject using a nebulizer. In some embodiments, the modified Cav-1 peptide or pharmaceutical formulation thereof is administered to the lungs of a subject using a dry powder inhaler. In some embodiments, the modified Cav-1 peptide or pharmaceutical formulation thereof is administered intranasally, intrabronchially, intrapleurally, intratracheally, or via inhalation to a subject.

In some embodiments, the modified Cav-1 peptide or pharmaceutical formulation thereof is administered to a subject in a single dose or in multiple doses. When multiple doses are administered, the doses may be separated from one another by, for example, one hour, three hours, six hours, eight hours, twelve hours, one day, two days, three days, four days, five days, six days, one week, two weeks, three weeks, one month, two months, three months, four months, five months, six months, one year, or any value or range therebetween. In some embodiments, the modified Cav-1 peptide or pharmaceutical formulation thereof is administered, for example, once every 2 weeks, once every 3 weeks, once every 4 weeks, once every 5 weeks, once every 6 weeks, once every 7 weeks, once every 8 weeks, once every 10 weeks, once every 15 weeks, once every 20 weeks, or more. It is to be understood that, for any particular subject, specific dosage regimens should be adjusted over time according to the individual need and the professional judgment of the person administering or supervising the administration of the formulations. For example, the dosage of the modified Cav-1 peptide or pharmaceutical formulation thereof can be increased if the lower dose does not provide sufficient therapeutic activity.

In some embodiments, the modified Cav-1 peptide or pharmaceutical formulation thereof is administered to a subject at a dose of about 0.0001 mg/kg to about 1,000 mg/kg. In some embodiments, the modified Cav-1 peptide or pharmaceutical formulation thereof is administered to a subject at a dose of about 0.0001 mg/kg to about 0.01 mg/kg. In some embodiments, the modified Cav-1 peptide or pharmaceutical formulation thereof is administered to a subject at a dose of about 0.01 mg/kg to about 1 mg/kg. In some embodiments, the modified Cav-1 peptide or pharmaceutical formulation thereof is administered to a subject at a dose of about 1 mg/kg to about 100 mg/kg. In some embodiments, the modified Cav-1 peptide or pharmaceutical formulation thereof is administered to a subject at a dose of about 1 mg/kg to about 50 mg/kg. In some embodiments, the modified Cav-1 peptide or pharmaceutical formulation thereof is administered to a subject at a dose of about 1 mg/kg to about 25 mg/kg. In some embodiments, the modified Cav-1 peptide or pharmaceutical formulation thereof is administered to a subject at a dose of about 1 mg/kg to about 10 mg/kg.

In some embodiments, the modified Cav-1 peptide or pharmaceutical formulation thereof is administered to a subject at a dose of about 10 mg/kg to about 25 mg/kg. In some embodiments, the modified Cav-1 peptide or pharmaceutical formulation thereof is administered to a subject at a dose of about 25 mg/kg to about 50 mg/kg. In some embodiments, the modified Cav-1 peptide or pharmaceutical formulation thereof is administered to a subject at a dose of about 50 mg/kg to about 75 mg/kg. In some embodiments, the modified Cav-1 peptide or pharmaceutical formulation thereof is administered to a subject at a dose of about 75 mg/kg to about 100 mg/kg. In some embodiments, the modified Cav-1 peptide or pharmaceutical formulation thereof is administered to a subject at a dose of about 0.0001 mg/kg, about 0.01 mg/kg, about 0.01 mg/kg, about 0.1 mg/kg, about 1 mg/kg, about 5 mg/kg, about 10 mg/kg, about 25 mg/kg, about 50 mg/kg, about 100 mg/kg, about 500 mg/kg, or about 1,000 mg/kg. In some embodiments, the modified Cav-1 peptide or pharmaceutical formulation thereof is administered to a subject at a dose of about 1 mg/kg to about 10 mg/kg. In some embodiments, the modified Cav-1 peptide or pharmaceutical formulation thereof is administered to a subject at a dose of about 2 mg/kg to 5 mg/kg. In some embodiments, the modified Cav-1 peptide or pharmaceutical formulation thereof is administered to a subject at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or any value or range therebetween. In some embodiments, the modified Cav-1 peptide or pharmaceutical formulation thereof is administered to a subject at a dose of about 1 mg/kg to about 5 mg/kg. In some embodiments, the modified Cav-1 peptide or pharmaceutical formulation thereof is administered to a subject at a dose of about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, or any value or range therebetween. In some embodiments, the modified Cav-1 peptide or pharmaceutical formulation thereof is administered to a subject at a dose of about 2.2 mg/kg.

In some embodiments, the total or complete dose of a modified Cav-1 peptide or pharmaceutical formulation thereof administered to a subject is between about 1 mg to about 100 mg, such as between about 20 mg to about 100 mg, between about 50 mg to about 100 mg, between about 10 mg to about 20 mg, between about 20 mg to about 40 mg, between about 50 mg to about 70 mg, or between about 80 mg to about 90 mg. In some embodiments, the total or complete dose of a modified Cav-1 peptide or pharmaceutical formulation thereof administered to a subject is about 1 mg, about 2 mg, about 3 mg, about 4 mg, about 5 mg, about 6 mg, about 7 mg, about 8 mg, about 9 mg, about 10 mg, about 12 mg, about 14 mg, about 16 mg, about 18 mg, about 20 mg, about 22 mg, about 24 mg, about 26 mg, about 28 mg, about 30 mg, about 32 mg, about 34 mg, about 36 mg, about 38 mg, about 40 mg, about 42 mg, about 44 mg, about 46 mg, about 48 mg, about 50 mg, about 52 mg, about 54 mg, about 56 mg, about 58 mg, about 60 mg, about 62 mg, about 64 mg, about 66 mg, about 68 mg, about 70 mg, about 72 mg, about 74 mg, about 76 mg, about 78 mg, about 80 mg, about 82 mg, about 84 mg, about 86 mg, about 88 mg, about 90 mg, about 92 mg, about 94 mg, about 96 mg, about 98 mg, about 100 mg, about 110 mg, about 120 mg, or any value or subranges therebetween.

In some embodiments, the modified Cav-1 peptide or pharmaceutical formulation thereof is prepared as an extended-release formulation. The term “extended release” herein refers to the ability to release an ingredient (i.e., the modified Cav-1 peptides) over a specific period of time. Without wishing to be bound by any theory, it is envisioned that the formulations of the present disclosure provide extended release of the modified Cav-1 peptides for specified periods, such as, but not limited to, about one week or more, about two weeks or more, about three weeks or more, about four weeks or more, about five weeks or more, about six weeks or more, about seven weeks or more, about eight weeks or more, about 12 weeks or more, about 16 weeks or more, about 20 weeks or more, about 24 weeks or more, about 28 weeks or more, about one month or more, about two months or more, about three months or more, about four months or more, about five months or more, about six months or more.

In some embodiments, dosages of the modified Cav-1 peptide or pharmaceutical formulation thereof for a particular subject are determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to a subject is sufficient to provide a beneficial therapeutic response in the subject over time, or, e.g., to reduce symptoms, or other appropriate activity, depending on the application. The dose is determined by the efficacy of the particular formulation, and the activity, stability and/or serum half-life of the modified Cav-1 peptides disclosed herein and the condition of the subject, as well as the body weight or surface area of the subject to be treated.

In some embodiments, a subject is administered a dose of the modified Cav-1 peptide or pharmaceutical formulation thereof once per day for the treatment or prevention of any one of the diseases or conditions described herein. In some embodiments, the subject is elderly or of advanced age. In some embodiments, the single dose is between about 0.2 mg/kg and about 250 mg/kg, such as between about 1 mg/kg to about 10 mg/kg, between about 10 mg/kg and about 25 mg/kg, between about 25 mg/kg to about 50 mg/kg, between about 50 mg/kg to about 75 mg/kg, between about 75 mg/kg to about 100 mg/kg, for example, via lung instillation (e.g., inhalation). Such a dose can be administered daily for anywhere from about 3 days to one or more weeks or at any frequency as disclosed herein. Chronic administration of the modified Cav-1 peptide or pharmaceutical formulation thereof is also possible, although the dose may need to be adjusted downward as is well-understood in the art. The foregoing ranges are, however, suggestive, as the number of variables in an individual treatment regime is large, and considerable excursions from these preferred values are expected.

In some embodiments, the modified Cav-1 peptide or pharmaceutical formulation thereof is administered continuously to a subject. For continuous administration, e.g., by a pump system such as an osmotic pump, a total dosage for a time course of about 1-2 weeks in the range of about 1 mg/kg to about 1 g/kg, about 20 to about 300 mg/kg, or about 50 to about 200 mg/kg. After such a continuous dosing regimen, the total concentration of the active compound can be in the range of about 0.5 μM to about 50 μM, or about 1 μM to about 10 μM.

In some embodiments, the modified Cav-1 peptide or pharmaceutical formulation thereof is administered on a routine schedule. As used herein, a routine schedule refers to a predetermined designated period of time. The routine schedule may encompass periods of time which are identical or which differ in length, as long as the schedule is predetermined. For instance, the routine schedule may involve administration once per day, twice per day, once every two days, once every three days, once every four days, once every five days, once every six days, once per week, once every two weeks, once every three weeks, once per month, once every two months, once every three months, once every six months, or any set number of days, weeks, or months there-between. In some embodiments, the modified Cav-1 peptide or pharmaceutical formulation thereof is administered on a twice daily basis for the first week, followed by a daily basis for several months. In some embodiments, the modified Cav-1 peptide or pharmaceutical formulation thereof is administered once per day. In some embodiments, the modified Cav-1 peptide or pharmaceutical formulation thereof is administered less than once per day, such as every other day, every third day, or once per week.

In some embodiments, the modified Cav-1 peptide formulation is administered to a subject for at least about 2 weeks, at least about 3 weeks, at least about 4 weeks, at least about 5 weeks, at least about 6 weeks, at least about 2 months, at least about 3 months, at least about 4 months, at least about 5 months, at least about 6 months, at least about 1 year, or any set number of weeks or months there-between. In some embodiments, the modified Cav-1 peptide formulation is administered to a subject with fibrosis for at least about 2 weeks. In some embodiments, the modified Cav-1 peptide formulation is administered to a subject with fibrosis for at least about 4 weeks.

In some embodiments, the modified Cav-1 peptide or pharmaceutical formulation thereof is provided in a unit dosage form (e.g., pre-divided dose), such as in a capsule, blister or a cartridge. In some embodiments, the unit dose comprises at least 1 mg of the modified Cav-1 peptide formulation, such as at least about 5 mg, at least about 10 mg, at least about 15 mg, or at least about 20 mg of the modified Cav-1 peptide formulation per dose. In some embodiments, the unit dose is about 1 mg to about 10 mg (e.g., about 5 mg) of the modified Cav-1 peptide formulation. In some embodiments, the unit dosage form does not comprise the administration or addition of any excipient and is merely used to hold the powder for inhalation (i.e., the capsule, blister, or cartridge is not administered). In some embodiments, more than one of the unit dose forms is administered to a subject. For example, in the case of a dry powder inhaler, the modified Cav-1 peptide formulation is provided in unit dose capsules and more than one unit dose capsules (e.g., 3-4) can be administered to a subject by inhalation. In some embodiments, the modified Cav-1 peptide formulation is administered in a high emitted dose, such as at least about 10 mg, at least about 15 mg, or at least about 20 mg. In some embodiments, administration of milled modified Cav-1 peptide formulation results in a high fine particle dose into the deep lung such as greater than about 5 mg. In some embodiments, the fine particle dose into the deep lung is at least about 10 mg or at least about 15 mg. In some embodiments, the particle dose is produced from 1, 2, 3, 4 or 5 or more capsules comprising doses of a peptide of the embodiments. In some embodiments, the fine particle dose is at least about 50%, at least about 60%, at least about 65%, at least about 70%, at least about 75%, or at least about 80% of the emitted dose.

In some embodiments, the modified Cav-1 peptide or pharmaceutical formulation thereof is administered in combination, simultaneously or sequentially with at least one additional therapeutic agent for the treatment or prevention of a disease or disorder of the kidneys in a subject. In some embodiments, the disease is chronic kidney disease. The additional therapeutic agents include, but are not limited to, angiotensin-converting enzyme (ACE) inhibitors, such as, Capoten® (captopril), Vasotec® (enalapril), Monopril® (fosinopril), Prinivil® or Zestril® (lisinopril), or Altace® (ramipril); angiotensin II receptor (ARB) inhibitors, such as Edarbi® (azilsartan), Teveten® (eprosartan), Avapro® (irbesartan), Cozaar® (losartan), Benicar® (olmesartan), or Diovan® (valsartan); Farxiga® (dapagliflozin); Aranesp® (darbepoetin alpha); and/or Procrit® or Epogen® (erythropoietin).

In some embodiments, the modified Cav-1 peptide or pharmaceutical formulation thereof is administered in combination with dialysis for the treatment of a disease or disorder of the kidneys in a subject. In some embodiments, the modified Cav-1 peptide or pharmaceutical formulation thereof is administered in combination with dialysis for the treatment of chronic kidney disease. In some embodiments, the dialysis is hemodialysis. In some embodiments, the dialysis is peritoneal dialysis.

In some embodiments, the modified Cav-1 peptide or pharmaceutical formulation thereof is administered in combination, simultaneously or sequentially with at least one additional therapeutic agent for lung fibrosis. The additional therapeutic agents include, but are not limited to, a non-steroidal anti-inflammatory drug (NSAID), steroid, disease-modifying antirheumatic drug (DMARD), immunosuppressive, biologic response modulators, bronchodilator or antifibrotic agent such as pirfenedone, an agent whose antifibrotic mechanism of action is not fully understood but may involve blockade of TGF-beta, nintedanib, a broad tyrosine kinase blocker or any other antifibrotic agent. Suitable NSAIDS are selected from the non-selective cyclooxygenase (COX)-inhibitors acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lornoxicam and nimesulide and the pharmaceutically acceptable salts thereof, the selective COX 2-inhibitors meloxicam, celecoxib and rofecoxib and the pharmaceutically acceptable salts thereof. Suitable steroids are prednisone, prednisolone, methylprednisolone, dexamethasone, budenoside, fluocortolone and triamcinolone. Suitable DMARDs are sulfasalazine, olsalazine, chloroquin, gold derivatives (auranofin), D-penicillamine and cytostatics such as methotrexate and cyclophosphamide. Suitable immunsuppressives are cyclosporine A and derivatives thereof, mycophenolatemofetil, FK 506 (also known as tacrolimus and fugimycin), muromonab-CD3 (Orthoclone OKT-3@), anti-thymocyte globulin (ATG), 15-desoxyspergualin, mizoribine, misoprostol, rapamycin, reflunomide and azathioprine. Suitable biologic response modifiers are interferon β, anti-TNF-α antibody (etanercept), IL-10, anti-CD3 antibody or anti-CD25 antibody. Suitable bronchodilators are ipratropiumbromide, oxytropiumbromide, tiotropiumbromide, epinephrinehydrochloride, salbutamole, terbutalinsulfate, fenoterolhydrobromide, salmeterole and formoterole. In such combinations each active ingredient can be administered either in accordance with its usual dosage range or a dose below its usual dosage range. The dosage for the combined NSAIDs, steroids, DMARDs, immunosuppressives and biologic response modifiers is appropriately 1/50 of the lowest dose normally recommended up to 1/1 of the normally recommended dosage, 1/20 to ½, or 1/10 to ⅕. The normally recommended dose for the combined drug should be understood to be the dose disclosed, for example, in Rote Liste® 2002, Editio Cantor Verlag Aulendorf, Germany, or in Physician's Desk Reference.

In some embodiments, the modified Cav-1 peptide or pharmaceutical formulation thereof is administered in combination, simultaneously or sequentially with at least one additional therapeutic agent to treat the pathogen or pathogen-induced lung injury. Additional therapeutic agents include, but are not limited to, chloroquine, hydroxychloroquine, type I interferon, anti-virals, antibiotics, remdesivir, favipiravir, lopinavir, ritonavir, nirmatrelvir, baricitinib, and molnupiravir.

Hydroxychloroquine is a chemical derivative of chloroquine which features a hydroxyethyl group instead of an ethyl group. Hydroxychloroquine has been classified as an effective anti-malarial medication and has shown efficacy in treating systemic lupus erythematosus as well as rheumatoid arthritis and Sjögren's Syndrome. While hydroxychloroquine has been known for some time to increase lysosomal pH in antigen presenting cells, its mechanism of action in inflammatory conditions has been only recently elucidated and involves blocking the activation of toll-like receptors to on plasmacytoid dendritic cells. Hydroxychloroquine has shown efficacy in treating RNA viruses, including hepatitis C. Hydroxychloroquine may be administered at a dose of 600 mg per day.

Human type I interferons (IFNs) are a large subgroup of interferon proteins that help regulate the activity of the immune system. The mammalian types are designated IFN-α (alpha), IFN-β (beta), IFN-κ (kappa), IFN-δ(delta), IFN-ε (epsilon), IFN-τ (tau), IFN-ω (omega), and IFN-ζ ((zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments can be fused with other peptides and proteins such as immunoglobulins and other cytokines. Interferon-alpha and interferon-beta conjugates may represent, for example, a formulation comprising interferon-beta coupled to a non-naturally occurring polymer comprising a polyalkylene glycol moiety. Preferred interferon compounds include Roferon® (interferon alpha-2a), Intron® (interferon alpha-2b), Alferon® (interferon alpha-n3), Infergen® (interferon alfacon-1), Omniferon® (interferon alpha), interferon alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable formulations, interferon-alpha as a topical formulation, Roferon® (interferon alpha-2a) analogues, Intron® (interferon alpha-2b) analogues, Alferon® (interferon alpha-n3) analogues, and Infergen® (interferon alfacon-1) analogues, Omniferon® (interferon alpha) analogues, interferon alfacon-1 analogues, interferon beta, Avonex™ (interferon beta-1a), Betaseron™ (interferon beta-1b), Betaferon™ (interferon beta-1b), Rebif™ (interferon beta-1a), interferon-beta analogues, pegylated interferon-beta, polymerized interferon-beta, dimerized interferon-beta, interferon-beta conjugated to carriers, interferon-beta as oral inhalant, interferon-beta as an injectable formulation, interferon-beta as a topical formulation, Avonex™ (interferon beta-1a) analogues, Betaseron™ (interferon beta-1b) analogues, Betaferon™ (interferon beta-1b) analogues, and Rebif™ (interferon beta-1a) analogues. Alternatively, agents that induce interferon-alpha or interferon-beta production or mimic the action of interferon-alpha or interferon-beta may also be employed. Interferon inducers include tilorone, poly(I)-poly(C), imiquimod, cridanimod, bropirimine.

In some embodiments, the modified Cav-1 peptide or pharmaceutical formulation thereof comprises different types of carriers depending on whether the formulation is to be administered in solid, liquid, or aerosol form, and whether it needs to be sterile for the route of administration, such as injection.

In some embodiments, the modified Cav-1 peptide or pharmaceutical formulation thereof is administered intravenously, intrathecally, intradermally, transdermally, intrathecally, intraarterially, intraperitoneally, intranasally, intravaginally, intrarectally, intramuscularly, subcutaneously, mucosally, orally, topically, locally, by inhalation (e.g., inhalation of a nebulized or dry powder formulation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). The choice of injection volume and needle size may be chosen by the person of ordinary skill in the art based on site of injection, syringeability and injectability, which includes considering the viscosity of the solution or suspension to be injected and drug concentration, pH, and osmolality. In some instances, the particle size of the active agent can be chosen in order to provide a desired rate of dissolution upon administration (e.g., by subcutaneous injection).

In some embodiments, the formulation as disclosed herein is administered intravenously, intramuscularly, or subcutaneously. In some embodiments, the formulation as disclosed herein is administered by injection. In some embodiments, the formulation as disclosed herein is sustained release, controlled release, or extended release formulation.

In some embodiments, the formulation is an inhalable modified Cav-1 peptide formulations. Administration via inhalation includes, but is not limited to, use of an inhaler or nebulizer.

In some embodiments, the modified Cav-1 peptides or pharmaceutical formulations thereof are formulated into a formulation in a free base, neutral, or salt form. Pharmaceutically acceptable salts include the acid addition salts, e.g., those formed with the free amino groups of a proteinaceous formulation, or which are formed with inorganic acids, such as, for example, hydrochloric or phosphoric acids, or such organic acids as acetic, oxalic, tartaric, or mandelic acid. Salts formed with the free carboxyl groups can also be derived from inorganic bases, such as, for example, sodium, potassium, ammonium, calcium, or ferric hydroxides; or such organic bases as isopropylamine, trimethylamine, histidine, or procaine. Upon formulation, solutions will be administered in a manner compatible with the dosage formulation and in such amount as is therapeutically effective. The formulations are easily administered in a variety of dosage forms, such as formulated for parenteral administrations, such as injectable solutions, or aerosols for delivery to the lungs, or formulated for alimentary administrations, such as drug release capsules and the like.

In some embodiments, the modified Cav-1 peptide or pharmaceutical formulation thereof comprises a pharmaceutically acceptable carrier with or without an inert diluent. The carrier should be assimilable and includes liquid, semi-solid, i.e., pastes, or solid carriers. Except insofar as any conventional media, agent, diluent, or carrier is detrimental to the recipient or to the therapeutic effectiveness of a formulation contained therein, its use in administrable formulation for use in practicing the methods is appropriate. Examples of carriers or diluents include fats, oils, water, saline solutions, lipids, liposomes, resins, binders, fillers, and the like, or combinations thereof. In some embodiments, the modified Cav-1 peptide or pharmaceutical formulation thereof comprises one or more antioxidants to retard oxidation of one or more components in the formulation. Additionally, the prevention of the action of microorganisms can be brought about by preservatives, such as various antibacterial and antifungal agents, including but not limited to parabens (e.g., methylparabens, propylparabens), chlorobutanol, phenol, sorbic acid, thimerosal or combinations thereof.

In some embodiments, the modified Cav-1 peptide or pharmaceutical formulation thereof is combined or mixed thoroughly with a semi-solid or solid carrier. The mixing can be carried out in any convenient manner, such as grinding. Stabilizing agents can be also added in the mixing process in order to protect the formulation from loss of therapeutic activity, e.g., denaturation in the stomach. Examples of stabilizers include, but are not limited to, buffers, amino acids, such as glycine and lysine, carbohydrates or lyoprotectants, such as dextrose, mannose, galactose, fructose, lactose, sucrose, maltose, sorbitol, mannitol, etc.

In some embodiments, the modified Cav-1 peptide or pharmaceutical formulation thereof comprises one or more surfactants. Surfactants used in accordance with the disclosed methods include ionic and non-ionic surfactants. Representative non-ionic surfactants include polysorbates such as TWEEN®-20 and TWEEN-80@surfactants (ICI Americas Inc. of Bridgewater, N.J.); poloxamers (e.g., poloxamer 188); anionic and nonionic surfactants such as TRITON® surfactants (Sigma of St. Louis, Mo.); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palnidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; cationic or quaternary phospholipid surfactants such as MONAQUAT™ surfactants (Mona Industries Inc. of Paterson, N.J.); polyethyl glycol; polypropyl glycol; block copolymers of ethylene and propylene glycol such as PLURONIC® surfactants (BASF of Mt. Olive, N.J.); oligo (ethylene oxide) alkyl ethers; alkyl (thio) glucosides, alkyl maltosides; and phospholipids. In some embodiments, the one or more surfactants are present in the pharmaceutical formulation in an amount from about 0.01% to about 0.5% (weight of surfactant relative to total weight of other solid components of the formulation; “w/w”), from about 0.03% to about 0.5% (w/w), from about 0.05% to about 0.5% (w/w), or from about 0.1% to about 0.5% (w/w). In some embodiments, the modified Cav-1 peptide or pharmaceutical formulation thereof is essentially free of non-ionic surfactants or essentially free of all surfactants.

With respect to the therapeutic methods of the invention, it is not intended that the administration of the modified Cav-1 peptides or pharmaceutical formulations thereof disclosed herein be limited to a particular mode of administration, dosage, or frequency of dosing. The present invention contemplates all modes of administration, including intramuscular, intravenous, intraperitoneal, intravesicular, intraarticular, intralesional, subcutaneous, or any other route sufficient to provide a dose adequate to treat the disease or disorder. The modified Cav-1 peptide or pharmaceutical formulation thereof may be administered to the patient in a single dose or in multiple doses. When multiple doses are administered, the doses may be separated from one another by, for example, one hour, three hours, six hours, eight hours, twelve hours, one day, two days, three days, four days, five days, six days, one week, two weeks, three weeks, one month, two months, three months, four months, five months, six months, one year, or any value or range therebetween. In some embodiments, the modified Cav-1 peptide or pharmaceutical formulation thereof is administered, for example, once every 2 weeks, once every 3 weeks, once every 4 weeks, once every 5 weeks, once every 6 weeks, once every 7 weeks, once every 8 weeks, once every 10 weeks, once every 15 weeks, once every 20 weeks, or more. It is to be understood that, for any particular subject, specific dosage regimes should be adjusted over time according to the individual need and the professional judgment of the person administering or supervising the administration of the formulation. For example, the dosage of the modified Cav-1 peptide or pharmaceutical formulation thereof can be increased if the lower dose does not provide sufficient therapeutic activity.

### EXAMPLES

The disclosure is further described in detail by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1. SAIB Based Modified Cav-1 Peptide Formulation

A SAIB based modified Cav-1 peptide (APi2355, SEQ ID NO: 8) formulation was prepared. Approximately 34.0 mg micronized APi2355 was weighed into a 1 mL syringe. Separately approximately 567 mg of 80% w/w SAIB in NMP solution was weighed into a separate 1 mL syringe. A Luer-to-Luer connection was placed between the 2 syringes and mixing was performed until a homogenous white suspension was formed (30 mg APi2355/400 mg SAIB). Similarly, 10 mg APi2355/500 mg SAIB, 30 mg APi2355/500 mg SAIB, and 50 mg APi2355/500 mg SAIB suspensions were formed.

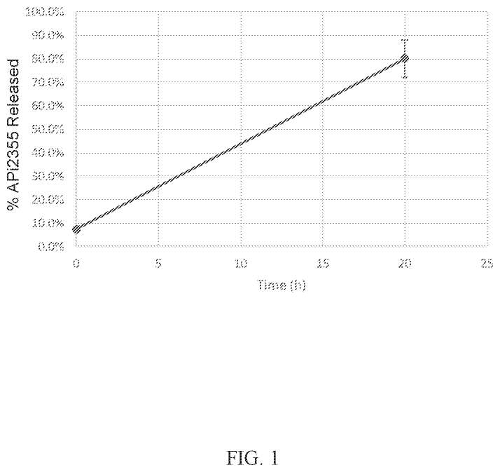

Approximately 150 mg of the 30 mg APi2355/400 mg SAIB suspension was added to about 35 mL of phosphate-buffered saline (PBS) and placed on an oscillation shaker at 37° C. at 150 rpm. A 1 mL sample was removed for testing at various time points and replaced with fresh prewarmed PBS. Samples were filtered and analyzed by HPLC for % release of APi2355. The in vitro release kinetics parameters for 30 mg APi2355/5400 mg SAIB formulation were determined, as shown in FIG. 1.

### Example 2. Pharmacokinetics of the Modified Cav-1 Peptide SAIB Formulation

The plasma pharmacokinetics of the Cav-1 peptide (APi2355) SAIB formulation prepared according to Example 1 (5.66% w/w APi2355 75.47% w/w SAIB and 18.87% w/w NMP to provide 7% w/w APi2355 to 93% w/w SAIB) were assessed over 42 days after a single-dose subcutaneous administration to male Sprague Dawley rats. The rats (n=6) received one of three batches of the APi2355 SAIB formulations prepared according to Example 1 at a dose of 2.0 mg/kg of APi2355 in 0.033 mL/kg (concentration 60.0 mg/mL). Control group of rats (n=2) received an injection of vehicle formulation containing no APi2355.

### Experimental Procedures

Animals were assigned to groups by a stratified randomization scheme designed to achieve similar group mean body weights. The animals were acclimated to their designated housing for at least 5 days before the day dosing. All formulations were administered in a single dose by dorsal subcutaneous (SC) injection on day 0. Whole blood samples (40-50 μl) were collected from all animals in APi2355 treated group at pre-dose and 1 h, 2 h, 4 h, 8 h, 1 day, 2 days, 4 days, 7 days, 10 days, 14 days, 21 days, 29 days, and 35 days post-treatment. Whole blood samples (40-50 μl, by tail nick) were collected from all animals in control group at pre-dose and at 1 h and 1 day post-treatment. Blood was collected into K₂EDTA tubes. Tubes were mixed gently after blood collection, chilled and centrifuged at 3000 rpm and 4° C., within 30 minutes upon collection. The resultant plasma was separated, transferred to uniquely labeled polypropylene tubes and frozen at −20° C. Plasma and tissue samples were analyzed for concentration of APi2355 using a qualified analytical procedure for LC/MS/MS.

Terminal whole blood samples (maximal volume by cardiac stick) and tissues were collected from all animals on day 42 post-treatment. Tissues included tissues from heart, liver, kidney, lung, and injection site skin patches. Additionally, lungs were processed for bronchial lavage samples. Tissue samples were analyzed for APi2355 by LC/MS/MS.

### Pharmacokinetic Results

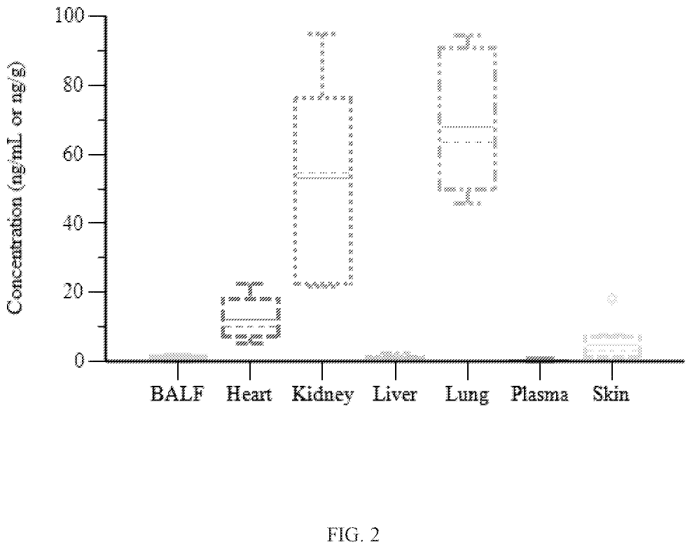

All concentration values of APi2355 in the control group and at the predose time were below the lower limit of quantitation (<0.3 ng/mL). The concentration-time profiles of APi2355 in rat plasma for APi2355 treated group were determined, as presented in Table 4. As shown in FIG. 2 and Table 5, the mean concentration profiles of APi2355 in various tissues were also determined. Table 6 summarizes mean pharmacokinetic exposure profiles of APi2355 in rat plasma measured following administration of the SAIB formulation.

TABLE 4
APi2355 plasma concentrations (ng/mL) over time (post-treatment)

| | 1 | 2 | 4 | 8 | 1 | 2 | 4 | 7 | 10 | 14 | 22 | 29 | 35 | 42 |
|---|---|---|---|---|---|---|---|---|---|---|---|---|---|---|
| | hr | hr | hr | hr | day | day | day | day | day | day | day | day | day | day |
| Rat 1 | 109.02 | 37.62 | 328.27 | 80.69 | 20.55 | 2.54 | 1.29 | 2.40 | 10.43 | 7.00 | 2.49 | 0.85 | 0.78 | 0.15 |
| Rat 2 | 101.53 | 317.55 | 164.91 | 75.60 | 12.72 | 8.72 | 2.17 | 2.14 | 5.54 | 1.83 | 5.68 | 0.65 | 0.10 | 0.00 |
| Rat 3 | 84.70 | 539.82 | 97.42 | 47.06 | 19.85 | 14.40 | 1.84 | 5.18 | 2.38 | 1.64 | 1.98 | 0.58 | 0.72 | 0.04 |
| Rat 4 | 557.93 | 497.89 | 52.01 | 39.24 | 25.11 | 6.90 | 1.41 | 1.44 | 1.58 | 3.04 | 1.93 | 0.16 | 4.20 | 0.00 |
| Rat 5 | 202.00 | 884.98 | 26.95 | 23.90 | 10.32 | 6.13 | 4.65 | 15.91 | 2.09 | 1.66 | 1.68 | 0.98 | 0.81 | 0.88 |
| Rat 6 | 232.90 | 111.43 | 102.25 | 54.20 | 11.86 | 3.87 | 3.40 | 5.07 | 2.04 | 1.24 | 1.87 | 0.50 | 1.13 | 0.29 |
| Mean | 214.68 | 398.21 | 128.64 | 53.45 | 16.73 | 7.09 | 2.46 | 5.36 | 4.01 | 2.73 | 2.61 | 0.62 | 1.29 | 0.23 |
| SD | 178.42 | 311.68 | 108.68 | 21.68 | 5.92 | 4.20 | 1.31 | 5.40 | 3.45 | 2.18 | 1.53 | 0.29 | 1.47 | 0.34 |

TABLE 5
APi2335 concentrations in various tissues at 42 days post treatment

| | BALF | Heart | Liver | Lung | Kidney | Skin |
|---|---|---|---|---|---|---|
| | (ng/mL) | (ng/g) | (ng/g) | (ng/g) | (ng/g) | (ng/g) |
| Rat 1 | 0.66 | 5.21 | 0.61 | 50.70 | 61.59 | 18.32 |
| Rat 2 | 0.62 | 22.64 | 0.96 | 68.57 | 94.61 | 0.83 |
| Rat 3 | 0.42 | 17.27 | 2.47 | 90.24 | 21.73 | 3.89 |
| Rat 4 | 1.96 | 8.97 | 1.38 | 45.72 | 22.41 | 0.32 |
| Rat 5 | 2.00 | 7.50 | 0.67 | 94.48 | 47.94 | 4.01 |
| Rat 6 | 0.27 | 11.05 | 1.30 | 58.55 | 70.84 | 2.32 |
| Mean | 0.99 | 12.11 | 1.23 | 68.04 | 53.19 | 4.95 |
| SD | 0.78 | 6.59 | 0.68 | 20.40 | 28.50 | 6.72 |

BALF: bronchoalveolar lavage fluid

TABLE 6
Pharmacokinetic parameters

| | | | | | | | | | % |
|---|---|---|---|---|---|---|---|---|---|
| | | | | | | | | | AUC |
| | Tₘₐₓ | Cₘₐₓ | Tₗₐₛₜ | Cₗₐₛₜ | AUC₀₋₁₄ | AUC₀₋₂₂ | AUCₗₐₛₜ | AUC_{inf} | Extrap |
| | (day) | (ng/mL) | (day) | (ng/mL) | (day*ng/mL) | (day*ng/mL) | (day*ng/mL) | (day*ng/mL) | (%) |
| Mean | 0.0831 | 477 | 39.7 | 0.942 | 126 | 146 | 164 | 183 | 9.00 |
| (median) | (0.0830) | | | | | | | | |
| SD | 0.0454 | 238 | 3.61 | 1.63 | 19.0 | 25.4 | 26.4 | 38.0 | 13.4 |

Tₘₐₓ: The time after dosing at which the maximum concentration was observed
Cₘₐₓ: The maximum observed concentration measured after dosing.
Tₗₐₛₜ: The time after dosing at which the last quantifiable concentration was observed
Cₗₐₛₜ: Last measurable concentration above the limit of quantitation
AUC₀₋₁₄: The area under the concentration-time curve from the start of dose administration to day 14
AUC₀₋₂₂: The area under the concentration-time curve from the start of dose administration to day 22
AUCₗₐₛₜ: The area under the concentration-time curve from the start of dose administration to the last observed quantifiable concentration calculated using the log/linear trapezoidal method.
AUC_{inf}: Area under the concentration-time curve from time 0 extrapolated to infinity, calculated as AUClast + Clast/λz
% AUC Extrap: Percentage of the area under the concentration-time curve extrapolated beyond the last quantifiable plasma concentration

Pharmacokinetic clearance and volume of distribution measured following administration of the SAIB formulation are summarized in Table 7.

TABLE 7
Pharmacokinetic clearance and volume of distribution parameters

| | | | | Half- | | | |
|---|---|---|---|---|---|---|---|
| | Points | | Rsq | life | MRTₗₐₛₜ | V_{z}/F | CL/F |
| | λz | Span | adj. | (day) | (day) | (mL/kg) | (mL/day/kg) |
| Mean | 6.17 | 3.75 | 0.323 | 9.24 | 7.82 | 133000 | 10000 |
| SD | 1.37 | 1.80 | 0.296 | 6.92 | 0.867 | 111000 | 2500 |

T_{1/2}: Terminal half-life
MRTₗₐₛₜ: Mean residence time from time zero to the time of last quantifiable concentration
V_{z}/F: Apparent volume of distribution during the terminal phase
CL/F: Apparent plasma clearance of drug
λz: Terminal rate constant
Rsq: adj: Adjusted coefficient of determination

### Pharmacokinetic Profile

After single subcutaneous administration, APi2355 was absorbed from the SAIB formulation with median Tₘₐₓ values of 0.083 days and the mean Cₘₐₓ value of 477 ng/mL post injection. APi2355 was detectable in 4 out of 6 rats in the plasma following administration. The average concentration at the last quantifiable time point was 0.942 ng/mL. As shown in Table 6, the AUC₀₋₁₄ mean value of 126 day*ng/mL, AUC₀₋₂₂ mean value of 146 day*ng/mL, and AUC_{inf} mean value of 183 day*ng/mL were observed.

The extrapolated AUC of AUCi_{nf} was <20%, therefore the time points used for evaluation of APi2355 concentrations adequately described the systemic exposure profiles and certain PK parameters such as AUC_{inf} are believed to be accurate. The Rsq adj. values wear generally <0.8 due to variability over the span, which was generally >3-fold the half-life. The concentration of APi2355 varied significantly over the span, which led to lower Rsq adj. The half-life values exhibited a mean value of 9.24 days. The mean MRTₗₐₛₜ value for the SAIB formulation was 7.82 days. Approximately ⅔ of the total exposure of APi2355 occurred in the first 3 weeks after SC administration.

### Numbered Embodiments

Embodiment 1. A formulation, comprising: a) a polypeptide comprising an amino acid sequence having a core sequence of FTTFTVT; and b) a sucrose acetate isobutyrate (SAIB).
Embodiment 2. The formulation of embodiment 1, further comprising a first solvent.
Embodiment 3. The formulation of embodiment 2, wherein the first solvent is selected from N-methylpyrrolidone (NMP), anhydrous ethanol, or ethyl acetate.
Embodiment 4. The formulation of any one of embodiments 1-3, wherein the SAIB is present in the first solvent in about 50% w/w to about 95% w/w.
Embodiment 5. The formulation of any one of embodiments 1-4, wherein the SAIB is present in the first solvent in about 70% w/w to about 90% w/w.
Embodiment 6. The formulation of any one of embodiments 1-5, wherein the SAIB is present in the first solvent in about 80% w/w.
Embodiment 7. The formulation of any one of embodiments 1-6, wherein the first solvent is NMP or ethyl acetate.
Embodiment 8. The formulation of any one of embodiments 1-7, wherein the polypeptide is micronized.
Embodiment 9. The formulation of any one of embodiments 1-8, wherein the polypeptide has a mean particle size in the range of about 0.5 μm to about 100 μm.
Embodiment 10. The formulation of any one of embodiments 1-9, wherein the polypeptide has a mean particle size in the range of about 1 m to about 5 μm.
Embodiment 11. The formulation of any one of embodiments 1-10, wherein the formulation is in the form of an emulsion, a solution, microspheres, nanoparticles, nanospheres, implants, or gels.
Embodiment 12. The formulation of any one of embodiments 1-10, wherein the formulation is a suspension.
Embodiment 13. The formulation of any one of embodiments 1-12 further comprising a second solvent.
Embodiment 14. The formulation of embodiment 13, wherein the second solvent is sterile water, phosphate-buffered saline (PBS), or any pharmaceutically acceptable carrier.
Embodiment 15. The formulation of any one of embodiments 1-14, wherein the formulation comprises the polypeptide and the SAIB at a weight ratio of about 1:50 to about 1:1.
Embodiment 16. The formulation of embodiment 15, wherein the weight ratio is about 1:30 to about 1:1.
Embodiment 17. The formulation of any one of embodiments 1-16, wherein the formulation releases at least about 1% to about 30% of the polypeptide by four hours under physiological conditions.
Embodiment 18. The formulation of any one of embodiments 1-16, wherein the formulation releases at least about 5% to about 25% of the polypeptide by four hours under physiological conditions.
Embodiment 19. The formulation of any one of embodiments 1-18, wherein the polypeptide comprises ASFTTFTVTK.
Embodiment 20. The formulation of any one of embodiments 1-19, wherein the polypeptide comprises: a) at least one amino acid added to the N-terminus; b) at least one amino acid added to the C-terminus; or c) at least one amino acid added to the N-terminus and the C-terminus.
Embodiment 21. The formulation of any one of embodiments 1-20, wherein the polypeptide consists of 20 or less amino acids.
Embodiment 22. The formulation of any one of embodiments 1-21, wherein the addition made within 5 amino acids of each terminus has an amino acid sequence that is less than 80% identical to a contiguous amino acid sequence of SEQ ID NO:1.
Embodiment 23. The formulation of embodiment 22, wherein the addition made within 5 amino acids of each terminus has an amino acid sequence that is less than 60% identical to a contiguous amino acid sequence of SEQ ID NO: 1.
Embodiment 24. The formulation of embodiment 22, wherein the addition made within 5 amino acids of each terminus has an amino acid sequence that is less than 40% identical to a contiguous amino acid sequence of SEQ ID NO: 1.
Embodiment 25. The formulation of embodiment 22, wherein the addition made within 5 amino acids of each terminus has an amino acid sequence that is less than 20% identical to a contiguous amino acid sequence of SEQ ID NO: 1.
Embodiment 26. The formulation of any one of embodiments 1-25, wherein the peptide sequence comprises: a) the polypeptide comprises L-amino acids; b) the polypeptide comprises D-amino acids; or c) the polypeptide comprises both L- and D-amino acids.
Embodiment 27. The formulation of any one of embodiments 1-26, wherein: a) the polypeptide comprises at least one non-standard amino acid; or b) the polypeptide comprises two non-standard amino acids.
Embodiment 28. The formulation of embodiment 27, wherein the non-standard amino acid is ornithine.
Embodiment 29. The formulation of any one of embodiments 1-28, wherein the polypeptide further comprises: a) an N-terminal modification; b) a C-terminal modification; or c) an N- and C-terminal modification.
Embodiment 30. The formulation of embodiment 29, wherein: the N-terminal modification is acylation; and/or the C-terminal modification is amidation.
Embodiment 31. The formulation of any one of embodiments 1-30, wherein the polypeptide comprises the amino acid sequence of KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10).
Embodiment 32. The formulation of any one of embodiments 1-31, wherein the polypeptide consists of an amino acid sequence selected from KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10).
Embodiment 33. The formulation of any one of embodiments 1-32, wherein the polypeptide consists of an amino acid sequence of Ac-aaEGKASFTTFTVTKGSaa-NH2 (SEQ ID NO: 8).
Embodiment 34. A method of treating a disease in a subject in need thereof, comprising administering to the subject a pharmaceutically effective amount of the formulation of any one of embodiments 1-33.
Embodiment 35. The method of embodiment 34, wherein the disease is fibrosis.
Embodiment 36. The method of embodiment 35, wherein the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.
Embodiment 37. The method of embodiment 36, wherein the interstitial lung disease is idiopathic pulmonary fibrosis, familial pulmonary fibrosis, idiopathic nonspecific interstitial pneumonia, conventional interstitial pneumonia, cryptogenic organizing pneumonia, or sarcoidosis.
Embodiment 38. The method of embodiment 37, wherein the interstitial lung disease is idiopathic pulmonary fibrosis.
Embodiment 39. The method of embodiment 34, wherein the disease is a kidney disease or disorder.
Embodiment 40. The method of embodiment 39, wherein the kidney disease or disorder is selected from the group consisting of chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, granulomatosis with polyangiitis, or acute kidney injury.
Embodiment 41. The method of embodiment 40, wherein the kidney disease or disorder is kidney fibrosis.
Embodiment 42. The method of embodiment 40, wherein the kidney disease or disorder is Alport syndrome.
Embodiment 43. The method of any one of embodiments 34-42, wherein the administration is intraocular, intradermal, transdermal, intramuscular, or subcutaneous administration.
Embodiment 44. The method of any one of embodiments 34-43, wherein the subject is a human.
Embodiment 45. The method of any one of embodiments 34-44, wherein the formulation releases the polypeptide for at least 7 days following administration of a single dose.
Embodiment 46. The method of any one of embodiments 34-44, wherein the formulation releases the polypeptide for at least 21 days following administration of a single dose.
Embodiment 47. The method of any one of embodiments 34-44, wherein the formulation releases the polypeptide for at least 28 days following administration of a single dose.
Embodiment 48. The method of any one of embodiments 34-47, wherein the formulation is administered to the subject at a dose of about 0.01 mg/kg to about 250 mg/kg.
Embodiment 49. The method of embodiment 48, wherein the formulation is administered to the subject at a dose of about 0.05 mg/kg to about 50 mg/kg.

What is claimed is:

1. A formulation, comprising:
a) a polypeptide comprising an amino acid sequence having a core sequence of FTTFTVT; and
b) a sucrose acetate isobutyrate (SAIB).

2. The formulation of claim 1, further comprising a first solvent.

3. The formulation of claim 2, wherein the first solvent is selected from N-methylpyrrolidone (NMP), anhydrous ethanol, or ethyl acetate.

4. The formulation of any one of claims 1-3, wherein the SAIB is present in the first solvent in about 50% w/w to about 95% w/w.

5. The formulation of any one of claims 1-4, wherein the SAIB is present in the first solvent in about 70% w/w to about 90% w/w.

6. The formulation of any one of claims 1-5, wherein the SAIB is present in the first solvent in about 80% w/w.

7. The formulation of any one of claims 1-6, wherein the first solvent is NP or ethyl acetate.

8. The formulation of any one of claims 1-7, wherein the polypeptide is micronized.

9. The formulation of any one of claims 1-8, wherein the polypeptide has a mean particle size in the range of about 0.5 μm to about 100 μm.

10. The formulation of any one of claims 1-9, wherein the polypeptide has a mean particle size in the range of about 1 μm to about 5 μm.

11. The formulation of any one of claims 1-10, wherein the formulation is in the form of an emulsion, a solution, microspheres, nanoparticles, nanospheres, implants, or gels.

12. The formulation of any one of claims 1-10, wherein the formulation is a suspension.

13. The formulation of any one of claims 1-12 further comprising a second solvent.

14. The formulation of claim 13, wherein the second solvent is sterile water, phosphate-buffered saline (PBS), or any pharmaceutically acceptable carrier.

15. The formulation of any one of claims 1-14, wherein the formulation comprises the polypeptide and the SAB at a weight ratio of about 1:50 to about 1:1.

16. The formulation of claim 15, wherein the weight ratio is about 1:30 to about 1:1.

17. The formulation of any one of claims 1-16, wherein the formulation releases at least about 1% to about 30% of the polypeptide by four hours under physiological conditions.

18. The formulation of any one of claims 1-16, wherein the formulation releases at least about 5% to about 25% of the polypeptide by four hours under physiological conditions.

19. The formulation of any one of claims 1-18, wherein the polypeptide comprises ASFTTFTVTK.

20. The formulation of any one of claims 1-19, wherein the polypeptide comprises:
a) at least one amino acid added to the N-terminus;
b) at least one amino acid added to the C-terminus; or
c) at least one amino acid added to the N-terminus and the C-terminus.

21. The formulation of any one of claims 1-20, wherein the polypeptide consists of 20 or less amino acids.

22. The formulation of any one of claims 1-21, wherein the addition made within 5 amino acids of each terminus has an amino acid sequence that is less than 80% identical to a contiguous amino acid sequence of SEQ ID NO:1.

23. The formulation of claim 22, wherein the addition made within 5 amino acids of each terminus has an amino acid sequence that is less than 60% identical to a contiguous amino acid sequence of SEQ ID NO: 1.

24. The formulation of claim 22, wherein the addition made within 5 amino acids of each terminus has an amino acid sequence that is less than 40% identical to a contiguous amino acid sequence of SEQ ID NO: 1.

25. The formulation of claim 22, wherein the addition made within 5 amino acids of each terminus has an amino acid sequence that is less than 20% identical to a contiguous amino acid sequence of SEQ ID NO: 1.

26. The formulation of any one of claims 1-25, wherein the peptide sequence comprises:
a) the polypeptide comprises L-amino acids;
b) the polypeptide comprises D-amino acids; or
c) the polypeptide comprises both L- and D-amino acids.

27. The formulation of any one of claims 1-26, wherein:
a) the polypeptide comprises at least one non-standard amino acid; or
b) the polypeptide comprises two non-standard amino acids.

28. The formulation of claim 27, wherein the non-standard amino acid is ornithine.

29. The formulation of any one of claims 1-28, wherein the polypeptide further comprises:
a) an N-terminal modification;
b) a C-terminal modification; or
c) an N- and C-terminal modification.

30. The formulation of claim 29, wherein:
the N-terminal modification is acylation; and/or
the C-terminal modification is amidation.

31. The formulation of any one of claims 1-30, wherein the polypeptide comprises the amino acid sequence of KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10).

32. The formulation of any one of claims 1-31, wherein the polypeptide consists of an amino acid sequence selected from KASFTTFTVTKGS (SEQ ID NO: 4), KASFTTFTVTKGS-NH2 (SEQ ID NO: 5), aaEGKASFTTFTVTKGSaa (SEQ ID NO: 6), aaEGKASFTTFTVTKGSaa-NH2 (SEQ ID NO: 7), Ac-aaEGKASFTTFTVTKGSaa-NH2 (SEQ ID NO: 8), OASFTTFTVTOS (SEQ ID NO: 9), or OASFTTFTVTOS-NH2 (SEQ ID NO: 10).

33. The formulation of any one of claims 1-32, wherein the polypeptide consists of an amino acid sequence of Ac-aaEGKASFTTFTVTKGSaa-NH2 (SEQ ID NO: 8).

34. A method of treating a disease in a subject in need thereof, comprising administering to the subject a pharmaceutically effective amount of the formulation of any one of claims 1-33.

35. The method of claim 34, wherein the disease is fibrosis.

36. The method of claim 35, wherein the fibrosis is interstitial lung disease, liver fibrosis, renal fibrosis, skin fibrosis, glomerulonephritis, systemic sclerosis, cardiac fibrosis, myocardial fibrosis, kidney fibrosis, hepatic cirrhosis, renal sclerosis, arteriosclerosis, macular degeneration, ocular scarring, cataracts, retinal and vitreal retinopathy, Grave's ophthalmopathy, neurofibromatosis, scleroderma, glioblastoma, keloids and hypertrophic scarring, peritoneal fibrotic disease, chronic obstructive pulmonary disease, post-operative fibroids, diabetic nephropathy, gynecological cancer, myeloproliferative syndrome, myeloid leukemia, myelodysplastic syndrome, inflammatory bowel disease, non-alcoholic fatty liver disease, fibrosarcoma, rheumatoid arthritis, non-alcoholic steatohepatitis, Alport syndrome, or chronic COVID syndrome.

37. The method of claim 36, wherein the interstitial lung disease is idiopathic pulmonary fibrosis, familial pulmonary fibrosis, idiopathic nonspecific interstitial pneumonia, conventional interstitial pneumonia, cryptogenic organizing pneumonia, or sarcoidosis.

38. The method of claim 37, wherein the interstitial lung disease is idiopathic pulmonary fibrosis.

39. The method of claim 34, wherein the disease is a kidney disease or disorder.

40. The method of claim 39, wherein the kidney disease or disorder is selected from the group consisting of chronic kidney disease, end-stage renal disease, glomerulonephritis, focal segmental glomerulosclerosis, kidney fibrosis, polycystic kidney disease, IgA nephropathy, lupus nephritis, nephrotic syndrome, Alport syndrome, amyloidosis, Goodpasture syndrome, granulomatosis with polyangiitis, or acute kidney injury.

41. The method of claim 40, wherein the kidney disease or disorder is kidney fibrosis.

42. The method of claim 40, wherein the kidney disease or disorder is Alport syndrome.

43. The method of any one of claims 34-42, wherein the administration is intraocular, intradermal, transdermal, intramuscular, or subcutaneous administration.

44. The method of any one of claims 34-43, wherein the subject is a human.

45. The method of any one of claims 34-44, wherein the formulation releases the polypeptide for at least 7 days following administration of a single dose.

46. The method of any one of claims 34-44, wherein the formulation releases the polypeptide for at least 21 days following administration of a single dose.

47. The method of any one of claims 34-44, wherein the formulation releases the polypeptide for at least 28 days following administration of a single dose.

48. The method of any one of claims 34-47, wherein the formulation is administered to the subject at a dose of about 0.01 mg/kg to about 250 mg/kg.

49. The method of claim 48, wherein the formulation is administered to the subject at a dose of about 0.05 mg/kg to about 50 mg/kg.


SEQUENCE LISTING (118 sequences)

SEQ ID NO: 1 - protein, 178 aa, Homo sapiens
  REGION 1..178: note = Caveolin-1
MSGGKYVDSEGHLYTVPIREQGNIYKPNNKAMADELSEKQVYDAHTKEIDLVNRDPKHLNDDVVKIDFEDVIAEPEGTHSFDGIWKASFTTFTVTKYWFYRLLSALFGIPMALIWGIYFAILSFLHIWAVVPCIKSFLIEIQCISRVYSIYVHTVCDPLFEAVGKIFSNVRINLQKEI

SEQ ID NO: 2 - protein, 20 aa, synthetic construct
  REGION 1..20: note = Modified Caveolin-1 peptide
DGIWKASFTTFTVTKYWFYR

SEQ ID NO: 3 - protein, 7 aa, synthetic construct
  REGION 1..7: note = Modified Caveolin-1 peptide
FTTFTVT

SEQ ID NO: 4 - protein, 13 aa, synthetic construct
  REGION 1..13: note = Modified Caveolin-1 peptide
KASFTTFTVTKGS

SEQ ID NO: 5 - protein, 13 aa, synthetic construct
  REGION 1..13: note = Modified Caveolin-1 peptide
  SITE 13: note = the C-terminus is amidated
KASFTTFTVTKGS

SEQ ID NO: 6 - protein, 19 aa, synthetic construct
  REGION 1..19: note = Modified Caveolin-1 peptide
  SITE 1..2: note = D-Alanine
  SITE 18..19: note = D-Alanine
AAEGKASFTTFTVTKGSAA

SEQ ID NO: 7 - protein, 19 aa, synthetic construct
  REGION 1..19: note = Modified Caveolin-1 peptide
  SITE 1..2: note = D-alanine
  SITE 18..19: note = D-alanine
  SITE 13: note = the C-terminus is amidated
AAEGKASFTTFTVTKGSAA

SEQ ID NO: 8 - protein, 19 aa, synthetic construct
  REGION 1..19: note = Modified Caveolin-1 peptide
  SITE 1: note = the C-terminus is acetylated
  SITE 1..2: note = D-alanine
  SITE 18..19: note = D-alanine
  SITE 19: note = the C-terminus is amidated
AAEGKASFTTFTVTKGSAA

SEQ ID NO: 9 - protein, 12 aa, synthetic construct
  REGION 1..12: note = Modified Caveolin-1 peptide
  SITE 1: note = Orn
  SITE 11: note = Orn
XASFTTFTVTXS

SEQ ID NO: 10 - protein, 12 aa, synthetic construct
  REGION 1..12: note = Modified Caveolin-1 peptide
  SITE 1: note = Orn
  SITE 11: note = Orn
  SITE 11: note = the C-terminus is amidated
XASFTTFTVTXS

SEQ ID NO: 11 - protein, 7 aa, synthetic construct
  REGION 1..7: note = Modified Caveolin-1 peptide
  SITE 7: note = the C-terminus is amidated
FTTFTVT

SEQ ID NO: 12 - protein, 8 aa, synthetic construct
  REGION 1..8: note = Modified Caveolin-1 peptide
  SITE 8: note = the C-terminus is amidated
FTTFTVTK

SEQ ID NO: 13 - protein, 11 aa, synthetic construct
  REGION 1..11: note = Modified Caveolin-1 peptide
  SITE 11: note = the C-terminus is amidated
KASFTTFTVTK

SEQ ID NO: 14 - protein, 11 aa, synthetic construct
  REGION 1..11: note = Modified Caveolin-1 peptide
  SITE 1: note = the N-terminus is acetylated
  SITE 11: note = the C-terminus is amidated
KASFTTFTVTK

SEQ ID NO: 15 - protein, 11 aa, synthetic construct
  REGION 1..11: note = Modified Caveolin-1 peptide
  SITE 1: note = Orn
  SITE 11: note = the C-terminus is amidated
XASFTTFTVTK

SEQ ID NO: 16 - protein, 11 aa, synthetic construct
  REGION 1..11: note = Modified Caveolin-1 peptide
  SITE 1: note = the N-terminus is acetylated
  SITE 1: note = Orn
  SITE 11: note = the C-terminus is amidated
XASFTTFTVTK

SEQ ID NO: 17 - protein, 13 aa, synthetic construct
  REGION 1..13: note = Modified Caveolin-1 peptide
  SITE 1: note = the N-terminus is acetylated
  SITE 13: note = the C-terminus is amidated
KASFTTFTVTKGS

SEQ ID NO: 18 - protein, 14 aa, synthetic construct
  REGION 1..14: note = Modified Caveolin-1 peptide
  SITE 14: note = the C-terminus is amidated
DSGKASFTTFTVTK

SEQ ID NO: 19 - protein, 14 aa, synthetic construct
  REGION 1..14: note = Modified Caveolin-1 peptide
  SITE 1: note = the N-terminus is acetylated
  SITE 14: note = the C-terminus is amidated
DSGKASFTTFTVTK

SEQ ID NO: 20 - protein, 12 aa, synthetic construct
  REGION 1..12: note = Modified Caveolin-1 peptide
  SITE 1: note = the N-terminus is acetylated
  SITE 1: note = Orn
  SITE 11: note = Orn
  SITE 12: note = the C-terminus is amidated
XASFTTFTVTXS

SEQ ID NO: 21 - protein, 9 aa, synthetic construct
  REGION 1..9: note = Modified Caveolin-1 peptide
ASFTTFTVT

SEQ ID NO: 22 - protein, 12 aa, synthetic construct
  REGION 1..12: note = Modified Caveolin-1 peptide
KASFTTFTVTKY

SEQ ID NO: 23 - protein, 12 aa, synthetic construct
  REGION 1..12: note = Modified Caveolin-1 peptide
IWKASFTTFTVT

SEQ ID NO: 24 - protein, 13 aa, synthetic construct
  REGION 1..13: note = Modified Caveolin-1 peptide
SFTTFTVTKYWFY

SEQ ID NO: 25 - protein, 13 aa, synthetic construct
  REGION 1..13: note = Modified Caveolin-1 peptide
KASFTTFTVTKYW

SEQ ID NO: 26 - protein, 13 aa, synthetic construct
  REGION 1..13: note = Modified Caveolin-1 peptide
IWKASFTTFTVTK

SEQ ID NO: 27 - protein, 14 aa, synthetic construct
  REGION 1..14: note = Modified Caveolin-1 peptide
FTTFTVTKYWFYRL

SEQ ID NO: 28 - protein, 14 aa, synthetic construct
  REGION 1..14: note = Modified Caveolin-1 peptide
ASFTTFTVTKYWFY

SEQ ID NO: 29 - protein, 14 aa, synthetic construct
  REGION 1..14: note = Modified Caveolin-1 peptide
WKASFTTFTVTKYW

SEQ ID NO: 30 - protein, 14 aa, synthetic construct
  REGION 1..14: note = Modified Caveolin-1 peptide
GIWKASFTTFTVTK

SEQ ID NO: 31 - protein, 15 aa, synthetic construct
  REGION 1..15: note = Modified Caveolin-1 peptide
FTTFTVTKYWFYRLL

SEQ ID NO: 32 - protein, 15 aa, synthetic construct
  REGION 1..15: note = Modified Caveolin-1 peptide
ASFTTFTVTKYWFYR

SEQ ID NO: 33 - protein, 15 aa, synthetic construct
  REGION 1..15: note = Modified Caveolin-1 peptide
WKASFTTFTVTKYWF

SEQ ID NO: 34 - protein, 15 aa, synthetic construct
  REGION 1..15: note = Modified Caveolin-1 peptide
GIWKASFTTFTVTKY

SEQ ID NO: 35 - protein, 15 aa, synthetic construct
  REGION 1..15: note = Modified Caveolin-1 peptide
FDGIWKASFTTFTVT

SEQ ID NO: 36 - protein, 16 aa, synthetic construct
  REGION 1..16: note = Modified Caveolin-1 peptide
SFTTFTVTKYWFYRLL

SEQ ID NO: 37 - protein, 16 aa, synthetic construct
  REGION 1..16: note = Modified Caveolin-1 peptide
KASFTTFTVTKYWFYR

SEQ ID NO: 38 - protein, 16 aa, synthetic construct
  REGION 1..16: note = Modified Caveolin-1 peptide
IWKASFTTFTVTKYWF

SEQ ID NO: 39 - protein, 16 aa, synthetic construct
  REGION 1..16: note = Modified Caveolin-1 peptide
DGIWKASFTTFTVTKY

SEQ ID NO: 40 - protein, 16 aa, synthetic construct
  REGION 1..16: note = Modified Caveolin-1 peptide
SFDGIWKASFTTFTVT

SEQ ID NO: 41 - protein, 17 aa, synthetic construct
  REGION 1..17: note = Modified Caveolin-1 peptide
SFTTFTVTKYWFYRLLS

SEQ ID NO: 42 - protein, 17 aa, synthetic construct
  REGION 1..17: note = Modified Caveolin-1 peptide
KASFTTFTVTKYWFYRL

SEQ ID NO: 43 - protein, 17 aa, synthetic construct
  REGION 1..17: note = Modified Caveolin-1 peptide
IWKASFTTFTVTKYWFY

SEQ ID NO: 44 - protein, 17 aa, synthetic construct
  REGION 1..17: note = Modified Caveolin-1 peptide
DGIWKASFTTFTVTKYW

SEQ ID NO: 45 - protein, 17 aa, synthetic construct
  REGION 1..17: note = Modified Caveolin-1 peptide
SFDGIWKASFTTFTVTK

SEQ ID NO: 46 - protein, 18 aa, synthetic construct
  REGION 1..18: note = Modified Caveolin-1 peptide
FTTFTVTKYWFYRLLSAL

SEQ ID NO: 47 - protein, 18 aa, synthetic construct
  REGION 1..18: note = Modified Caveolin-1 peptide
ASFTTFTVTKYWFYRLLS

SEQ ID NO: 48 - protein, 18 aa, synthetic construct
  REGION 1..18: note = Modified Caveolin-1 peptide
WKASFTTFTVTKYWFYRL

SEQ ID NO: 49 - protein, 18 aa, synthetic construct
  REGION 1..18: note = Modified Caveolin-1 peptide
GIWKASFTTFTVTKYWFY

SEQ ID NO: 50 - protein, 18 aa, synthetic construct
  REGION 1..18: note = Modified Caveolin-1 peptide
FDGIWKASFTTFTVTKYW

SEQ ID NO: 51 - protein, 18 aa, synthetic construct
  REGION 1..18: note = Modified Caveolin-1 peptide
HSFDGIWKASFTTFTVTK

SEQ ID NO: 52 - protein, 19 aa, synthetic construct
  REGION 1..19: note = Modified Caveolin-1 peptide
FTTFTVTKYWFYRLLSALF

SEQ ID NO: 53 - protein, 19 aa, synthetic construct
  REGION 1..19: note = Modified Caveolin-1 peptide
ASFTTFTVTKYWFYRLLSA

SEQ ID NO: 54 - protein, 19 aa, synthetic construct
  REGION 1..19: note = Modified Caveolin-1 peptide
WKASFTTFTVTKYWFYRLL

SEQ ID NO: 55 - protein, 19 aa, synthetic construct
  REGION 1..19: note = Modified Caveolin-1 peptide
GIWKASFTTFTVTKYWFYR

SEQ ID NO: 56 - protein, 19 aa, synthetic construct
  REGION 1..19: note = Modified Caveolin-1 peptide
FDGIWKASFTTFTVTKYWF

SEQ ID NO: 57 - protein, 19 aa, synthetic construct
  REGION 1..19: note = Modified Caveolin-1 peptide
HSFDGIWKASFTTFTVTKY

SEQ ID NO: 58 - protein, 19 aa, synthetic construct
  REGION 1..19: note = Modified Caveolin-1 peptide
GTHSFDGIWKASFTTFTVT

SEQ ID NO: 59 - protein, 8 aa, synthetic construct
  REGION 1..8: note = Modified Caveolin-1 peptide
FTTFTVTK

SEQ ID NO: 60 - protein, 8 aa, synthetic construct
  REGION 1..8: note = Modified Caveolin-1 peptide
SFTTFTVT

SEQ ID NO: 61 - protein, 9 aa, synthetic construct
  REGION 1..9: note = Modified Caveolin-1 peptide
FTTFTVTKY

SEQ ID NO: 62 - protein, 9 aa, synthetic construct
  REGION 1..9: note = Modified Caveolin-1 peptide
SFTTFTVTK

SEQ ID NO: 63 - protein, 9 aa, synthetic construct
  REGION 1..9: note = Modified Caveolin-1 peptide
ASFTTFTVT

SEQ ID NO: 64 - protein, 10 aa, synthetic construct
  REGION 1..10: note = Modified Caveolin-1 peptide
FTTFTVTKYW

SEQ ID NO: 65 - protein, 10 aa, synthetic construct
  REGION 1..10: note = Modified Caveolin-1 peptide
SFTTFTVTKY

SEQ ID NO: 66 - protein, 10 aa, synthetic construct
  REGION 1..10: note = Modified Caveolin-1 peptide
ASFTTFTVTK

SEQ ID NO: 67 - protein, 10 aa, synthetic construct
  REGION 1..10: note = Modified Caveolin-1 peptide
KASFTTFTVT

SEQ ID NO: 68 - protein, 11 aa, synthetic construct
  REGION 1..11: note = Modified Caveolin-1 peptide
FTTFTVTKYWF

SEQ ID NO: 69 - protein, 11 aa, synthetic construct
  REGION 1..11: note = Modified Caveolin-1 peptide
SFTTFTVTKYW

SEQ ID NO: 70 - protein, 11 aa, synthetic construct
  REGION 1..11: note = Modified Caveolin-1 peptide
ASFTTFTVTKY

SEQ ID NO: 71 - protein, 11 aa, synthetic construct
  REGION 1..11: note = Modified Caveolin-1 peptide
KASFTTFTVTK

SEQ ID NO: 72 - protein, 11 aa, synthetic construct
  REGION 1..11: note = Modified Caveolin-1 peptide
WKASFTTFTVT

SEQ ID NO: 73 - protein, 12 aa, synthetic construct
  REGION 1..12: note = Modified Caveolin-1 peptide
FTTFTVTKYWFY

SEQ ID NO: 74 - protein, 12 aa, synthetic construct
  REGION 1..12: note = Modified Caveolin-1 peptide
SFTTFTVTKYWF

SEQ ID NO: 75 - protein, 12 aa, synthetic construct
  REGION 1..12: note = Modified Caveolin-1 peptide
ASFTTFTVTKYW

SEQ ID NO: 76 - protein, 12 aa, synthetic construct
  REGION 1..12: note = Modified Caveolin-1 peptide
WKASFTTFTVTK

SEQ ID NO: 77 - protein, 13 aa, synthetic construct
  REGION 1..13: note = Modified Caveolin-1 peptide
FTTFTVTKYWFYR

SEQ ID NO: 78 - protein, 13 aa, synthetic construct
  REGION 1..13: note = Modified Caveolin-1 peptide
ASFTTFTVTKYWF

SEQ ID NO: 79 - protein, 13 aa, synthetic construct
  REGION 1..13: note = Modified Caveolin-1 peptide
WKASFTTFTVTKY

SEQ ID NO: 80 - protein, 13 aa, synthetic construct
  REGION 1..13: note = Modified Caveolin-1 peptide
GIWKASFTTFTVT

SEQ ID NO: 81 - protein, 14 aa, synthetic construct
  REGION 1..14: note = Modified Caveolin-1 peptide
SFTTFTVTKYWFYR

SEQ ID NO: 82 - protein, 14 aa, synthetic construct
  REGION 1..14: note = Modified Caveolin-1 peptide
KASFTTFTVTKYWF

SEQ ID NO: 83 - protein, 14 aa, synthetic construct
  REGION 1..14: note = Modified Caveolin-1 peptide
IWKASFTTFTVTKY

SEQ ID NO: 84 - protein, 14 aa, synthetic construct
  REGION 1..14: note = Modified Caveolin-1 peptide
DGIWKASFTTFTVT

SEQ ID NO: 85 - protein, 15 aa, synthetic construct
  REGION 1..15: note = Modified Caveolin-1 peptide
SFTTFTVTKYWFYRL

SEQ ID NO: 86 - protein, 15 aa, synthetic construct
  REGION 1..15: note = Modified Caveolin-1 peptide
KASFTTFTVTKYWFY

SEQ ID NO: 87 - protein, 15 aa, synthetic construct
  REGION 1..15: note = Modified Caveolin-1 peptide
IWKASFTTFTVTKYW

SEQ ID NO: 88 - protein, 15 aa, synthetic construct
  REGION 1..15: note = Modified Caveolin-1 peptide
DGIWKASFTTFTVTK

SEQ ID NO: 89 - protein, 16 aa, synthetic construct
  REGION 1..16: note = Modified Caveolin-1 peptide
FTTFTVTKYWFYRLLS

SEQ ID NO: 90 - protein, 16 aa, synthetic construct
  REGION 1..16: note = Modified Caveolin-1 peptide
ASFTTFTVTKYWFYRL

SEQ ID NO: 91 - protein, 16 aa, synthetic construct
  REGION 1..16: note = Modified Caveolin-1 peptide
WKASFTTFTVTKYWFY

SEQ ID NO: 92 - protein, 16 aa, synthetic construct
  REGION 1..16: note = Modified Caveolin-1 peptide
GIWKASFTTFTVTKYW

SEQ ID NO: 93 - protein, 16 aa, synthetic construct
  REGION 1..16: note = Modified Caveolin-1 peptide
FDGIWKASFTTFTVTK

SEQ ID NO: 94 - protein, 17 aa, synthetic construct
  REGION 1..17: note = Modified Caveolin-1 peptide
FTTFTVTKYWFYRLLSA

SEQ ID NO: 95 - protein, 17 aa, synthetic construct
  REGION 1..17: note = Modified Caveolin-1 peptide
ASFTTFTVTKYWFYRLL

SEQ ID NO: 96 - protein, 17 aa, synthetic construct
  REGION 1..17: note = Modified Caveolin-1 peptide
WKASFTTFTVTKYWFYR

SEQ ID NO: 97 - protein, 17 aa, synthetic construct
  REGION 1..17: note = Modified Caveolin-1 peptide
GIWKASFTTFTVTKYWF

SEQ ID NO: 98 - protein, 17 aa, synthetic construct
  REGION 1..17: note = Modified Caveolin-1 peptide
FDGIWKASFTTFTVTKY

SEQ ID NO: 99 - protein, 17 aa, synthetic construct
  REGION 1..17: note = Modified Caveolin-1 peptide
HSFDGIWKASFTTFTVT

SEQ ID NO: 100 - protein, 18 aa, synthetic construct
  REGION 1..18: note = Modified Caveolin-1 peptide
SFTTFTVTKYWFYRLLSA

SEQ ID NO: 101 - protein, 18 aa, synthetic construct
  REGION 1..18: note = Modified Caveolin-1 peptide
KASFTTFTVTKYWFYRLL

SEQ ID NO: 102 - protein, 18 aa, synthetic construct
  REGION 1..18: note = Modified Caveolin-1 peptide
IWKASFTTFTVTKYWFYR

SEQ ID NO: 103 - protein, 18 aa, synthetic construct
  REGION 1..18: note = Modified Caveolin-1 peptide
DGIWKASFTTFTVTKYWF

SEQ ID NO: 104 - protein, 18 aa, synthetic construct
  REGION 1..18: note = Modified Caveolin-1 peptide
SFDGIWKASFTTFTVTKY

SEQ ID NO: 105 - protein, 18 aa, synthetic construct
  REGION 1..18: note = Modified Caveolin-1 peptide
THSFDGIWKASFTTFTVT

SEQ ID NO: 106 - protein, 19 aa, synthetic construct
  REGION 1..19: note = Modified Caveolin-1 peptide
SFTTFTVTKYWFYRLLSAL

SEQ ID NO: 107 - protein, 19 aa, synthetic construct
  REGION 1..19: note = Modified Caveolin-1 peptide
KASFTTFTVTKYWFYRLLS

SEQ ID NO: 108 - protein, 19 aa, synthetic construct
  REGION 1..19: note = Modified Caveolin-1 peptide
IWKASFTTFTVTKYWFYRL

SEQ ID NO: 109 - protein, 19 aa, synthetic construct
  REGION 1..19: note = Modified Caveolin-1 peptide
DGIWKASFTTFTVTKYWFY

SEQ ID NO: 110 - protein, 19 aa, synthetic construct
  REGION 1..19: note = Modified Caveolin-1 peptide
SFDGIWKASFTTFTVTKYW

SEQ ID NO: 111 - protein, 19 aa, synthetic construct
  REGION 1..19: note = Modified Caveolin-1 peptide
THSFDGIWKASFTTFTVTK

SEQ ID NO: 112 - protein, 13 aa, synthetic construct
  REGION 1..13: note = Cell penetrating peptide
GRKKRRQRRRPPQ

SEQ ID NO: 113 - protein, 16 aa, synthetic construct
  REGION 1..16: note = Cell penetrating peptide
RQIKIWFQNRRMKWKK

SEQ ID NO: 114 - protein, 26 aa, synthetic construct
  REGION 1..26: note = Cell penetrating peptide
GIGAVLKVLTTGLPALISWIKRKRQQ

SEQ ID NO: 115 - protein, 10 aa, synthetic construct
  REGION 1..10: note = Cell penetrating peptide
TKIESLKEHG

SEQ ID NO: 116 - protein, 10 aa, synthetic construct
  REGION 1..10: note = Cell penetrating peptide
TQIENLKEKG

SEQ ID NO: 117 - protein, 26 aa, synthetic construct
  REGION 1..26: note = Cell penetrating peptide
AALEALAEALEALAEALEALAEAAAA

SEQ ID NO: 118 - protein, 25 aa, synthetic construct
  REGION 1..25: note = Cell penetrating peptide
GLFEAIEGFIENGWEGMIEGWYGCG